(19) United States
(12) Patent Application Publication
(10) Pub. No.: US 2003/0082757 A1
(43) Pub. Date: May 1, 2003

(54) Novel proteins and nucleic acids encoding same

(76) Inventors: Raymond J. Taupier, East Haven, CT (US); Catherine E. Burgess, Wethersfield, CT (US); Muralidhara Padigaru, Bronx, NY (US); Velizar T. Tchernev, Branford, CT (US); Vishnu S. Mishra, Gainesville, FL (US); Stacie J. Casman, Wallingford, CT (US); Robert A. Ballinger, Newington, CT (US); Corine A.M. Vernet, North Branford, CT (US); Li Li, Chesire, CT (US); Kimberly A. Spytek, New Haven, CT (US); David P. Andrew, Branford, CT (US); Peter S. Mezes, Old Lyme, CT (US)

Correspondence Address: Ivor R. Elrifi, Esq., Mintz, Levin, Cohn, Ferris,, Glovsky and Popeo, P.C., One Financial Center, Boston, MA, 02111, US

(21) Appl. No.: 09/779,679

(22) Filed: Feb. 8, 2001

Related U.S. Application Data
(60) Provisional application No. 60/181,045, filed on Feb. 8, 2000.

(51) Int. Cl. C12N 9/00; C07H 21/04; C12P 21/02; C12N 5/06

(52) U.S. Cl. 435/183; 435/69.1; 435/325; 435/320.1; 536/23.2

(57) ABSTRACT

Disclosed herein are acid sequences which encode polypeptides. Also disclosed are nucleic acid sequences, and antibodies which immunospecifically-bind to the polypeptide, as well as derivatives, variants, mutants, or fragments of the aforementioned polypeptide, polynucleotide, or antibody. The invention further discloses thereapeutic, diagnostic and research methods for diagnosis, treatment, and prevention of disorders involving any one of these novel human nucleic acids and proteins.

### RELATED APPLICATIONS

This application claims priority from Provisional Applications U.S.S.N. 60/181,045, filed Feb. 8, 2000; U.S.S.N. 60/183,191, filed Feb. 17, 2000; U.S.S.N. 60/180,929, filed Feb. 8, 2000; U.S.S.N. 60/219,758, filed Jul. 20, 2000; U.S.S.N. 60/181,339, filed Feb. 9, 2000; U.S.S.N. 60/181,344, filed Feb. 9, 2000; U.S.S.N. 60/221,341, filed Jul. 26, 2000; U.S.S.N. 60/181,392, filed Feb. 9, 2000; U.S.S.N. 60/219,585, filed Jul. 20, 2000; and U.S.S.N. 60/181,157 filed on Feb. 9, 2000; each of which is incorporated by reference in its entirety.

### BACKGROUND

The invention generally relates to novel GPCR1, GPCR2, GPCR3, GPCR4, GPCR5, GPCR6 and GPCR7 nucleic acids and polypeptides encoded therefrom. More specifically, the invention relates to nucleic acids encoding novel polypeptides, as well as vectors, host cells, antibodies, and recombinant methods for producing these nucleic acids and polypeptides.

### SUMMARY OF THE INVENTION

The invention is based in part upon the discovery of novel nucleic acid sequences encoding novel polypeptides. The disclosed GPCR1, GPCR2, GPCR3, GPCR4, GPCR5, GPCR6 and GPCR7 nucleic acids and polypeptides encoded therefrom, as well as derivatives, homologs, analogs and fragments thereof, will hereinafter be collectively designated as “GPCRX” nucleic acid or polypeptide sequences.

In one aspect, the invention provides an isolated GPCRX nucleic acid molecule encoding a GPCRX polypeptide that includes a nucleic acid sequence that has identity to the nucleic acids disclosed in SEQ ID NOS:1, 3, 5, 7, 9, 11, 13, 15, 17, 19, and 21. In some embodiments, the GPCRX nucleic acid molecule will hybridize under stringent conditions to a nucleic acid sequence complementary to a nucleic acid molecule that includes a protein-coding sequence of a GPCRX nucleic acid sequence. The invention also includes an isolated nucleic acid that encodes a GPCRX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, and 22. The nucleic acid can be, for example, a genomic DNA fragment or a cDNA molecule that includes the nucleic acid sequence of any of SEQ ID NOS: 1, 3, 5, 7, 9, 11, 13, 15, 17, 19, and 21.

Also included in the invention is an oligonucleotide, e.g., an oligonucleotide which includes at least 6 contiguous nucleotides of a GPCRX nucleic acid (e.g., SEQ ID NOS: 1, 3, 5, 7, 9, 11, 13, 15, 17, 19, and 21) or a complement of said oligonucleotide.

Also included in the invention are substantially purified GPCRX polypeptides (SEQ ID NO: 2, 4, 6, 8, 10, 12, 14, 16, 18, 20, and 22). In certain embodiments, the GPCRX polypeptides include an amino acid sequence that is substantially identical to the amino acid sequence of a human GPCRX polypeptide.

The invention also features antibodies that immunoselectively-binds to GPCRX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a GPCRX nucleic acid, a GPCRX polypeptide, or an antibody specific for a GPCRX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a GPCRX nucleic acid, under conditions allowing for expression of the GPCRX polypeptide encoded by the DNA. If desired, the GPCRX polypeptide can then be recovered.

In another aspect, the invention includes a method of detecting the presence of a GPCRX polypeptide in a sample. In the method, a sample is contacted with a compound that selectively binds to the polypeptide under conditions allowing for formation of a complex between the polypeptide and the compound. The complex is detected, if present, thereby identifying the GPCRX polypeptide within the sample.

The invention also includes methods to identify specific cell or tissue types based on their expression of a GPCRX.

Also included in the invention is a method of detecting the presence of a GPCRX nucleic acid molecule in a sample by contacting the sample with a GPCRX nucleic acid probe or primer, and detecting whether the nucleic acid probe or primer bound to a GPCRX nucleic acid molecule in the sample.

In a further aspect, the invention provides a method for modulating the activity of a GPCRX polypeptide by contacting a cell sample that includes the GPCRX polypeptide with a compound that binds to the GPCRX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a Therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The Therapeutic can be, e.g, a GPCRX nucleic acid, a GPCRX polypeptide, or a GPCRX-specific antibody, or biologically-active derivatives or fragments thereof.

The invention further includes a method for screening for a modulator of disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a GPCRX polypeptide and determining if the test compound binds to said GPCRX polypeptide. Binding of the test compound to the GPCRX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a GPCRX nucleic acid. Expression or activity of GPCRX polypeptide is then measured in the test animal, as is expression or activity of the protein in a control animal which recombinantly-expresses GPCRX polypeptide and is not at increased risk for the disorder or syndrome. Next, the expression of GPCRX polypeptide in both the test animal and the control animal is compared. A change in the activity of GPCRX polypeptide in the test animal relative to the control animal indicates the test compound is a modulator of latency of the disorder or syndrome.

In yet another aspect, the invention includes a method for determining the presence of or predisposition to a disease associated with altered levels of a GPCRX polypeptide, a GPCRX nucleic acid, or both, in a subject (e.g., a human subject). The method includes measuring the amount of the GPCRX polypeptide in a test sample from the subject and comparing the amount of the polypeptide in the test sample to the amount of the GPCRX polypeptide present in a control sample. An alteration in the level of the GPCRX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a GPCRX polypeptide, a GPCRX nucleic acid, or a GPCRX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION

The invention is based, in part, upon the discovery of novel nucleic acid sequences that encode novel polypeptides. The novel nucleic acids and their encoded polypeptides are referred to individually as GPCR1, GPCR2, GPCR3, GPCR4, GPCRS, GPCR6, and GPCR7. The nucleic acids, and their encoded polypeptides, are collectively designated herein as “GPCRX”.

The novel GPCRX nucleic acids of the invention include the nucleic acids whose sequences are provided in Tables 1A, 2A, 2C, 3A, 4A, 4C, 5A, 5C, 5E, 6A, and 7A inclusive (“Tables 1A -7A”), or a fragment, derivative, analog or homolog thereof. The novel GPCRX proteins of the invention include the protein fragments whose sequences are provided in Tables 1B, 1C, 2B, 2D, 3B, 4B, 4D, 5B, 5D, 5F, 6B, and 7B inclusive (“Tables 1B-7B”). The individual GPCRX nucleic acids and proteins are described below. Within the scope of this invention is a method of using these nucleic acids and peptides in the treatment or prevention of a disorder related to cell signaling or metabolic pathway modulation.

GPCR1

Novel GPCR1 is a G-protein coupled receptor (“GPCR”) protein related to the cysteinyl leukotriene receptor. GPCR1 maps to human chromosome 13. The GPCR1 nucleic acid of 1260 nucleotides is shown in Table 1A. The GPCR1 open reading frame (“ORF”) begins at one of two alternative ATG initiation codons, shown in bold in Table 1A. In one embodiment, the GPCR1 ORF begins with an initiation codon at nucleotides 105-107, and the encoded polypeptide is alternatively referred to herein as GPCR1a or as AL137118A. In another embodiment, the GPCR1 ORF begins with an ATG initiation codon at nucleotides 120-122, and the encoded polypeptide is alternatively referred to herein as GPCR1b or as CG54236-02. In either embodiment, the GPCR1 ORF terminates at a TAA codon at nucleotides 1143-1145. As shown in Table 1A, putative untranslated regions 5′ to the start codon and 3′ to the stop codon are underlined, and the start and stop codons are in bold letters.

TABLE 1A
GPCR1 nucleotide sequence (SEQ ID NO:1).

TGCTCCCTGTTTCATTAAAACCTAGAGAGATGTAATCAGTAAGCAAGAAGGAAAAAGGGAAATTCACAAAGTAACTTTTTGTC
TCTGTTTCTTTTTAACCCAGCATGGAGAGAAAATTTATGTCCTTGCAACCATCCATCTCCGTATCAGAAATGGAACCAAATGG
CACCTTCAGCAATAACAACAGCAGGAACTGCACAATTGAAAACTTCAAGAGAGAATTTTTCCCAATTGTATATCTGATAATAT
TTTTCTGGGGAGTCTTGGGAAATGGGTTGTCCATATATGTTTTCCTGCAGCCTTATAAGAAGTCCACATCTGTGAACGTTTTC
ATGCTAAATCTGGCCATTTCAGATCTCCTGTTCATAAGCACGCTTCCCTTCAGGGCTGACTATTATCTTAGAGGCTCCAATTG
GATATTTGGAGACCTGGCCTGCAGGATTATGTCTTATTCCTTGTATGTCAACATGTACAGCAGTATTTATTTCCTGACCGTGC
TGAGTGTTGTGCGTTTCCTGGCAATGGTTCACCCCTTTCGGCTTCTGCATGTCACCAGCATCAGGAGTGCCTGGATCCTCTGT
GGGATCATATGGATCCTTATCATGGCTTCCTCAATAATGCTCCTGGACAGTGGCTCTGAGCAGAACGGCAGTGTCACATCATG
CTTAGAGCTGAATCTCTATAAAATTGCTAAGCTGCAGACCATGAACTATATTGCCTTGGTGGTGGGCTGCCTGCTGCCATTTT
TCACACTCAGCATCTGTTATCTGCTGATCATTCGGGTTCTGTTAAAAGTGGAGGTCCCAGAATCGGGGCTGCGGGTTTCTCAC
AGGAAGGCACTGACCACCATCATCATCACCTTGATCATCTTCTTCTTGTGTTTCCTGCCCTATCACACACTGAGGACCGTCCA
CTTGACGACATGGAAAGTGGGTTTATGCAAAGACAGACTGCATAAAGCTTTGGTTATCACACTGGCCTTGGCAGCAGCCAATG
CCTGCTTCAATCCTCTGCTCTATTACTTTGCTGGGGAGAATTTTAAGGACAGACTAAAGTCTGCACTCAGAAAAGGCCATCCA
CAGAAGGCAAAGACAAAGTGTGTTTTCCCTGTTAGTGTGTGGTTGAGAAAGGAAACAAGAGTATAAGGAGCTCTTAGATGAGA
CCTGTTCTTGTATCCTTGTGTCCATCTTCATTCACTCATAGTCTCCAAATGACTTTGTATTTACATCACTCCCAACAAATGTT
GATTCTTAATATTTA

In one embodiment, the encoded GPCR1 protein is translated from nucleotides 105 through 1145 and has 346 amino acid residues, referred to as the GPCR1a protein. The GPCR1a protein was analyzed for signal peptide prediction and cellular localization. SignalP results predict that GPCR1a is cleaved between position 59 and 60 of SEQ ID NO:2, i.e, at the dash in the amino acid sequence GLS-IYV. Psort and Hydropathy profiles also predict that GPCR1 contains a signal peptide and is likely to be localized at the plasma membrane (certainty of 0.6000). The GPCR1a polypeptide sequence is presented in Table 1B using the one-letter amino acid code.

TABLE 1B
Encoded GPCR1a protein sequence (SEQ ID NO:2).

MERKFMSLQPSISVSEMEPNGTFSNNNSRNCTIENFKREFFPIVYLIIFFWGVLGNGLSIYVFLQPYKKSTSVNVFMLNLAIS
DLLFISTLPFRADYYLRGSNWIFGDLACRIMSYSLYVNMYSSIYFLTVLSVVRFLAMVHPFRLLHVTSIRSAWILCGIIWILI
MASSIMLLDSGSEQNGSVTSCLELNLYKIAKLQTMNYIALVVGCLLPFFTLSICYLLIIRVLLKVEVPESGLRVSHRKALTTI
IITLIIFFLCFLPYHTLRTVHLTTWKVGLCKDRLHKLVITLALAAANACFNPLLYYFAGENFKDRLKSALRKGHPQKAKTKC
VFPVSVWLRKETRV

In an alternative embodiment, an encoded GPCR1 protein referred to alternatively as the GPCR1b or CG54236-02 polypeptide is translated from nucleotides 120 through 1145 and has a polypeptide sequence of 341 amino acid residues. The predicted GPCR1b polypeptide sequence includes amino acids 5 through 346 of SEQ ID NO:2 and is presented in Table 1C using the one-letter code. The identical predicted signal cleavage site in GPCR1a occurs in GPCR1b between position 54 and 55 of the sequence shown in Table 1C.

TABLE 1C
Encoded GPCR1b protein sequence.

MSLQPSISVSEMEPNGTFSNNNSRNCTIENFKREFFPIVYLIIFFWGVLGNGLSIYVFLQPYKKSTSVNVFMLNLAISDLLFI
STLPFRADYYLRGSNWIFGDLACRIMSYSLYVNMYSSIYFLTVLSVVRFLAMVHPFRLLHVTSIRSAWILCGIIWILIMASSI
MLLDSGSEQNGSVTSCLELNLYKIAKLQTMNYIALVVGCLLPFFTLSICYLLIIRVLLKVEVPESGLRVSHRKALTTIIITLI
IFFLCFLPYHTLRTVHLTTWKVGLCKDRLHKALVITLALAAANACFNPLLYYFAGENFKDRLKSALRKGHPQKAKTKCVFPVS
VWLRKETRV

Unless specifically addressed as GPCR1a or GPCR1b, any reference to a GPCR1 polypeptide or nucleic acid is assumed to encompass all variants.

GPCR1 was initially identified with a TblastN analysis of a proprietary sequence file for a G-protein coupled receptor probe or homolog which was run against the Genomic Daily Files made available by GenBank. A proprietary software program (GenScan™) was used to further predict the nucleic acid sequence and the selection of exons. The resulting sequences were further modified by means of similarities using BLAST searches. The sequences were then manually corrected for apparent inconsistencies, thereby obtaining the sequences encoding the full-length protein.

In an analysis of sequence databases, it was found, for example, that the GPCR1 nucleic acid sequence has 269 of 422 bases (63%) identical to a Gallus gallus activated T cell-specific G protein-coupled receptor mRNA (GenBank Acc. No. L06109) (SEQ ID NO:23) shown in Table 1D. In all BLAST alignments herein, the “E-value” or “Expect” value is a numeric indication of the probability that the aligned sequences could have achieved their similarity to the BLAST query sequence by chance alone, within the database that was searched. For example, as shown in Table 1E, the probability that the subject (“Sbjct”) retrieved from the GPCR1 BLAST analysis, in this case the Gallus gallus activated T cell-specific G protein-coupled receptor mRNA, matched the Query GPCR1 sequence purely by chance is 1.3×10⁻¹³.

TABLE 1D
BLASTN of GPCR1 against activated T cell-specific GPCR

```
GENBANK-ID:CHKGPCR|acc:L06109 Gallus gallus activated T cell-specific
G protein-coupled receptor mRNA (SEQ ID NO:23); Length = 1438
            Score = 509 (76.4 bits), Expect = 1.3e−13
            Identities = 269/422 (63%); Strand = Plus/Plus
```

| | | |
|---|---|---|
| Query: | 186 AGCAGGAACTGCACAATTGAAAACTTCAAGA-G-AGAATTTTTCCCAAT-TGTATATCTG | 242 |
| | \|\|\| \|\|\|\|\|\| \| \| \|\|\| \|\|\| \| \| \| \| \| \|\|\| \| \| \|\|\| \| \|\| \| | |
| Sbjct: | 91 AGCTCTAACTGCTCCACTGAGGACTCCTTTAAGTACACTTTGTATGGCTGTGTCT-TCAG | 149 |
| Query: | 243 -ATAATATTTTTCTGGGGAGTCTTGGGAAA-TGGGTTGTCCATATATGTTTTC-CTGCAG | 299 |
| | \|\| \|\|\|\|\| \|\| \|\| \|\| \| \| \|\| \|\| \|\|\|\| \| \|\| \|\| \|\|\|\| \|\| | |
| Sbjct: | 150 CATGGTATTTGTCCTCGGCCTCATAGCCAACTGCGTTG-CTATCTACATTTTTACTTTTA | 208 |
| Query: | 300 CCTTATAAGAAGTCCACATCTGTGA--ACGTTTT-CATGCTAAATCTGGCCATTTCAGAT | 356 |
| | \| \|\| \|\| \|\|\| \| \| \| \| \|\| \|\|\| \| \|\|\|\|\|\| \|\|\| \|\|\|\| \|\| \|\| \|\| | |
| Sbjct: | 209 CATTG-AA--AGTGCGGAAC-GAGACCACGACGTACATGCTGAATTTGGCGATATCGGAC | 264 |
| Query: | 357 CTCCTGTTCATAAGCACGCTTCCCTTCAGGGCTGACTATTATCTTAGAGGCTCC-AATTG | 415 |
| | \|\| \|\|\|\|\| \| \|\|\| \| \|\|\|\|\|\|\|\|\| \| \|\|\|\|\|\| \|\|\| \| \|\| \|\| \|\| | |
| Sbjct: | 265 CTGCTGTTTGTGTTTACGTTGCCCTTCAGGA-T--CTATTA-CTTCGTGGTGAGGAACTG | 320 |
| Query: | 416 GATATTTGGAGACCTGGCCTGCAGGAT-TATGTCTTATTCCTTGTATGTCAACATGTACA | 474 |
| | \| \|\| \|\|\|\|\|\| \| \|\|\|\| \|\|\| \| \|\|\| \| \|\| \|\| \|\|\|\|\|\|\|\|\|\| | |
| Sbjct: | 321 GCCCTTCGGAGACGTTCTGTGCAAGATCTCCGTCACGCTG-TTCTACACCAACATGTACG | 379 |
| Query: | 475 GCAGTATT-TATTTCCTGACCGTGC-TGAGTGTTGTGCGTTTCCTGGCAATGGTTCACCC | 532 |
| | \| \|\| \|\|\| \|\|\|\| \|\|\|\|\|\|\| \|\|\| \| \|\| \|\| \| \|\| \|\|\|\|\|\|\|\| \|\| \|\| \|\|\|\|\| | |
| Sbjct: | 380 GGAGCATTCTATT-CCTGACC-TGCATCAGCGTGGATCCCTTCCTGGCCATAGTGCACCC | 437 |
| Query: | 533 CTTTCGGCT-TCTGCATGTCACCAGCATCAGGAGTGCCTGGATCCTCTGTGGGATCATAT | 591 |
| | \|\|\|\|\|\| \|\| \| \| \| \| \|\| \|\| \|\|\|\| \|\|\| \|\|\|\|\| \| \|\| \| \| \| \| | |
| Sbjct: | 438 CTTTCG-CTCTAAGACTCTTCGCACCAAAAGGAACGCCAGGATCGTGTGCGTGGCGGTGT | 496 |
| Query: | 592 GGATCCTTATCATGGC | 607 |
| | \|\|\|\|\| \| \|\|\|\| | |
| Sbjct: | 497 GGATCACCGTGCTGGC | 512 |

In addition, the GPCR1 nucleic acid sequence has a 100% homology across 1260 nucleotides to the Homo sapiens cysteinyl leukotriene CysLT2 receptor (SEQ ID NO:24), as shown in Table 1E. The GenBank XM_{—}007164 sequence (SEQ ID NO:24) was directly deposited to National Center for Biotechnology Information, NIH, Bethesda, Md. 20894, USA, and provided to the public on Nov. 16, 2000.

TABLE 1E
BLASTN of GPCR1 against CysLT2 receptor

ref|XM_007164 Homo sapiens cysteinyl leukotriene
CysLT2 receptor; cDNA: PSEC0146 from clone
PLACE1006979 (LOC57105), mRNA (SEQ ID NO:24)
Score = 2498 bits (1260), Expect = 0.0
Identities = 1260/1260 (100%); Strand = Plus/Plus

| | | | |
|---|---|---|---|
| Query: | 1 | tgctccctgtttcattaaaacctagagagatgtaatcagtaagcaagaaggaaaaaggga | 60 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 160 | tgctccctgtttcattaaaacctagagagatgtaatcagtaagcaagaaggaaaaaggga | 219 |
| Query: | 61 | aattcacaaagtaactttttgtgtctgtttctttttaacccagcatggegagaaaattta | 120 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 220 | aattcacaaagtaactttttgtgtctgtttctttttaacccagcatggagagaaaattta | 279 |
| Query: | 121 | tgtccttgcaaccatccatctccgtatcagaaatggaaccaaatggcaccttcagcaata | 180 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 280 | tgtccttgcaaccatccatctccgtatcagaaatggaaccaaatggcaccttcagcaata | 339 |
| Query: | 181 | acaacagcaggaactgcacaattgaaaacttcaagagagaatttttcccaattgtatatc | 240 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 340 | acaacagcaggaactgcacaattgaaaacttcaagagagaatttttcccaattgtatatc | 399 |
| Query: | 241 | tgataatatttttctggggagtcttgggaaatgggttgtccatatatgttttcctgcagc | 300 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 400 | tgataatatttttctggggagtcttgggaaatgggttgtccatatatgttttcctgcagc | 459 |
| Query: | 301 | cttataagaagtccacatctgtgaacgttttcatgctaaatctggccatttcagatctcc | 360 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 460 | cttataagaagtccacatctgtgaacgttttcatgctaaatctggccatttcagatctcc | 519 |
| Query: | 361 | tgttcataagcacgcttcccttcagggctgactattatcttagaggctccaattggatat | 420 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 520 | tgttcataagcacgcttcccttcagggctgactattatcttagaggctccaattggatat | 579 |
| Query: | 421 | ttggagacctggcctgcaggattatgtcttattccttgtatgtcaacatgtacagcagta | 480 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 580 | ttggagacctggcctgcaggattatgtcttattccttgtatgtcaacatgtacagcagta | 639 |
| Query: | 481 | tttatttcctgaccgtgctgagtgttgtgcgtttcctggcaatggttcacccctttcggc | 540 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 640 | tttatttcctgaccgtgctgagtgttgtgcgtttcctggcaatggttcacccctttcggc | 699 |
| Query: | 541 | ttctgcatgtcaccagcatcaggagtgcctggatcctctgtgggatcatatggatcctta | 600 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 700 | ttctgcatgtcaccagcatcaggagtgcctggatcctctgtgggatcatatggatcctta | 759 |
| Query: | 601 | tcatggcttcctcaataatgctcctggacagtggctctgagcagaacggcagtgtcacat | 660 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 760 | tcatggcttcctcaataatgctcctggacagtggctctgagcagaacggcagtgtcacat | 819 |
| Query: | 661 | catgcttagagctgaatctctataaaattgctaagctgcagaccatgaactatattgcct | 720 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 820 | catgcttagagctgaatctctataaaattgctaagctgcagaccatgaactatattgcct | 879 |
| Query: | 721 | tggtggtgggctgcctgctgccatttttcacactcagcatctgttatctgctgatcattc | 780 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 880 | tggtggtgggctgcctgctgccatttttcacactcagcatctgttatctgctgatcattc | 939 |
| Query: | 781 | gggttctgttaaaagtggaggtcccagaatcggggctgcgggtttctcacaggaaggcac | 840 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 940 | gggttctgttaaaagtggaggtcccagaatcggggctgcgggtttctcacaggaaggcac | 999 |
| Query: | 841 | tgaccaccatcatcatcaccttgatcatcttcttcttgtgtttcctgccctatcacacac | 900 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1000 | tgaccaccatcatcatcaccttgatcatcttcttcttgtgtttcctgccctatcacacac | 1059 |
| Query: | 901 | tgaggaccgtccacttgacgacatggaaagtgggtttatgcaaagacagactgcataaag | 960 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1060 | tgaggaccgtccacttgacgacatggaaagtgggtttatgcaaagacagactgcataaag | 1119 |
| Query: | 961 | ctttggttatcacactggccttggcagcagccaatgcctgcttcaatcctctgctctatt | 1020 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1120 | ctttggttatcacactggccttggcagcagccaatgcctgcttcaatcctctgctctatt | 1179 |
| Query: | 1021 | actttgctggggagaattttaaggacagactaaagtctgcactcagaaaaggccatccac | 1080 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1180 | actttgctggggagaattttaaggacagactaaagtctgcactcagaaaaggccatccac | 1239 |
| Query: | 1081 | agaaggcaaagacaaagtgtgttttccctgttagtgtgtggttgagaaaggaaacaagag | 1140 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1240 | agaaggcaaagacaaagtgtgttttccctgttagtgtgtggttgagaaaggaaacaagag | 1299 |
| Query: | 1141 | tataaggagctcttagatgagacctgttcttgtatccttgtgtccatcttcattcactca | 1200 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1300 | tataaggagctcttagatgagacctgttcttgtatccttgtgtccatcttcattcactca | 1359 |
| Query: | 1201 | tagtctccaaatgactttgtatttacatcactcccaacaaatgttgattcttaatattta | 1260 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1360 | tagtctccaaatgactttgtatttacatcactcccaacaaatgttgattcttaatattta | 1419 |

TABLE 1F
BLASTX of GPCR1a against P2Y-Like GPCR

>ptnr:TREMBLNEW-ACC:CAA73144 P2Y-Like G-Protein Coupled
Receptor-Homo sapiens (Human), 367 aa (SEQ ID NO:25)
Score = 477 (167.9 bits), Expect = 1.3e−44, P = 1.3e−44
Identities = 113/313 (36%), Positives = 177/313 (56%), Frame = +3.

| | | |
|---|---|---|
| Query: | 135 SISVSEMEPNG---TFSNNNSRNCTIEN-FKREFFPIVYLIIFFWGVLGNGLSIYVFLQP | 302 |
| | \|++ \|+ \| \| \|\| + \| \| + \| \|\|+ \| ++\|\| \|++++\|++ | |
| Sbjct: | 28 SMNGLEVAPPGLITNFSLATAEQCGQETPLENMLFASFYLLDFILALVGNTLALWLFIED | 87 |
| Query: | 303 YKKSTSVNVFMLNLAISDLLFISTLPFRADYYLRGSNWIFGDLACRIMSYSLYVNMYSSI | 482 |
| | +\| \| \|\|\|+++\|\|++\|\| + \|\| \| \|+ \|++\| \|\|++\|\|\|+ + \|+\|\|\|+\|\| | |
| Sbjct: | 88 HKSGTPANVFLMHLAVADLSCVLVLPTRLVYHFSGNHWPFGEIACRLTGFLFYLNMYASI | 147 |
| Query: | 483 YFLTVLSVVRFLAMVHPFRLLHVTSIRSAWILCGIIWILI-MASSIMLLDSGSEQNGSVT | 659 |
| | \|\|\|\| +\| \|\|\|\|+\|\|\| + \| + \| + \| +\|+++ +\| + +\|+ + \| | |
| Sbjct: | 148 YFLTCISADRFLAIVHPVKSLKLRRPLYAELACAFLWVVVAVAMAPLLVSPQTVQTNHTV | 207 |
| Query: | 660 SCLELNLYKIAKLQTMNYIALVVGCLLPFFTLSICYLLIIRVLLKVEVPESGLRVSHR-- | 833 |
| | \|\|+\| \|+ \| ++\| \| \|\| \| \|\|\|\|\|\|\| \| \|\|\|\| \| | |
| Sbjct: | 208 VCLQL--YR-EKASHHALVSLAVAFTFPFITTVTCYLLIIRSL------RQGLRVEKFLK | 258 |
| Query: | 834 -KALTTIIITLIIFFLCFLPYHTLRTVHLTTWKV-GL-CKDRLHKALV--ITLALAAANA | 998 |
| | \|\|+ \| \| \| \|\| +\|\|+\|\|\| \|+\|++ ++ \| \| + \|\| \|\| \| + \| | |
| Sbjct: | 259 TKAVRMIAIVLAIFLVCFVPYHVNRSVYVLHYRSHGASCATQRILALANRITSCLTSLNG | 318 |
| Query: | 999 CFNPLLYYFAGENFKDRLKSAL | 1064 |
| | +\|++\|+\| \| \|+ \| + \| | |
| Sbjct: | 319 ALDPIMYFFVAEKFRHALCNLL | 340 |

As shown in Table 1G, the GPCR1a protein was also found to have 346 of 346 amino acid residues (100%) identical to, and 346 of 346 residues (100%) positive with, the 346 amino acid sequence of Homo sapiens cysteinyl leukotriene CysLT2 receptor (ptnr:XP_{—}007164) (SEQ ID NO:26). The cysteinyl leukotriene CysLT2 receptor (SEQ ID NO:26) is the protein encoded by GenBank XM_{—}007164 sequence (SEQ ID NO:24), above, and was also directly deposited to National Center for Biotechnology Information, NIH, and made public on Nov. 16, 2000.

TABLE 1G
BLASTX of GPCR1a against CysLT2 receptor

ptnr:XP_007164 cysteinyl leukotriene CysLT2 receptor;
cDNA: PSEC0146 from clone PLACE1006979
[Homo sapiens] (SEQ ID NO:26); Length = 346
Score = 657 bits (1696), Expect = 0.0
Identities = 346/346 (100%), Positives = 346/346 (100%)

| | | |
|---|---|---|
| Query: 1 | NERKFMSLQPSISVSEMEPNGTFSNNNSRNCTIENFKREFFPIVYLIIFFWGVLGNGLSI | 60 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 1 | MERKFMSLQPSISVSEMEPNGTFSNNNSRNCTIENFKREFFPIVYLIIFFWGVLGNGLSI | 60 |
| Query: 61 | YVFLQPYKKSTSVNVFMLNLAISDLLFISTLPFRADYYLRGSNWIFGDLACRINSYSLYV | 120 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 61 | YVFLQPYKKSTSVNVFMLNLAISDLLFISTLPFRADYYLRGSNWIFGDLACRIMSYSLYV | 120 |
| Query: 121 | NMYSSIYFLTVLSVVRFLAMVHPFRLLHVTSIRSAWILCGIIWILINASSIMLLDSGSEQ | 180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 121 | NMYSSIYFLTVLSVVRFLAMVHPFRLLHVTSIRSAWILCGIIWILIMASSIMLLDSGSEQ | 180 |
| Query: 181 | NGSVTSCLELNLYKIAKLQTMNYIALVVGCLLPFFTLSICYLLIIRVLLKVEVPESGLRV | 240 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 181 | NGSVTSCLELNLYKIAKLQTMNYIALVVGCLLPFFTLSICYLLIIRVLLKVEVPESGLRV | 240 |
| Query: 241 | SHRKALTTIIITLIIFFLCFLPYHTLRTVHLTTWKVGLCKDRLHKALVITLALAAANACF | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 241 | SHRKALTTIIITLIIFFLCFLPYHTLRTVHLTTWKVGLCKDRLHKALVITLALAAANACF | 300 |
| Query: 301 | NPLLYYFAGENFKDRLKSALRKGHPQKAKTKCVFPVSVWLRKETRV | 346 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 301 | NPLLYYFAGENFKDRLKSALRKGHPQKAXTKCVFPVSVWLRKETRV | 346 |

A ClustalW analysis comparing the protein of the invention with related protein sequences is given in Table 1H, with GPCR1 shown on line 2. In the ClustalW alignment of the GPCR1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

TABLE 1H
ClustalW Analysis of GPCR1

1) patp_W75799_Human, SEQ ID NO:27
2) (AL137118A) Novel GPCR1a, SEQ ID NO:2
3) (sptr-ACC:P34996) P2Y Purinoceptor 1 (ATP Receptor) (P2Y1) (Purinergic Receptor)
Gallus gallus (Chicken), 362 aa, SEQ ID NO:28
4) (STREMBL-ACC:CAA73144) P2Y-like G-Protein Coupled Receptor Homo sapiens (Human), 367 aa., SEQ ID NO:25

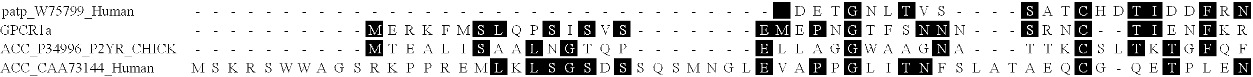

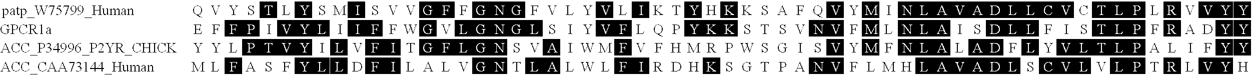

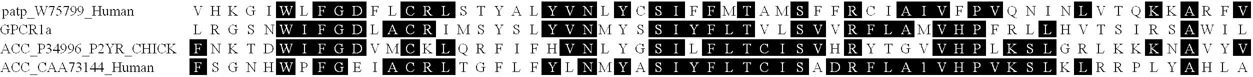

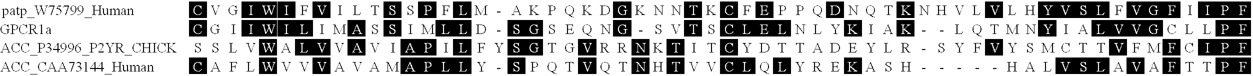

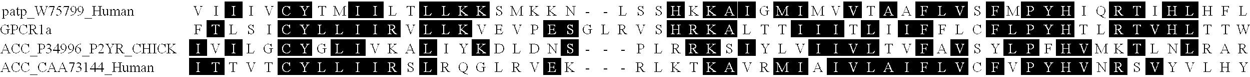

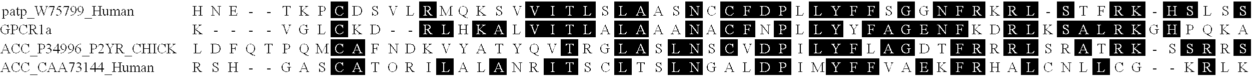

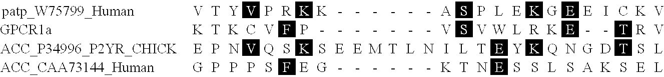

The presence of identifiable domains in GPCR1, as well as all other GPCRX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for GPCR1 as disclosed in Table 1I, were collected from the Conserved Domain Database (CDD) with Reverse Position Specific BLAST analyses. This BLAST analysis software samples domains found in the Smart and Pfam collections. For Table 1I and all successive DOMAIN sequence alignments, fully conserved single residues are indicated by (*) and “strong” semi-conserved residues are indicated by (:). The “strong” group of conserved amino acid residues may be any one of the following groups of amino acids: STA, NEQK, NHQK, NDEQ, QHRK, MILV, MILF, HY, FYW.

Table 1I lists the statistics and domain description from DOMAIN analysis results against GPCR 1. The region from amino acid residue 63 through 247 (numbered with respect to SEQ ID NO:2) most probably (E=3×10⁻³⁰) contains a “seven transmembrane receptor (rhodopsin family) fragment” domain, aligned here with residues 1- 177 of the 7tm₁₃1 entry (SEQ ID NO:29) of the Pfamn database. This indicates that the GPCR1 sequence has properties similar to those of other proteins known to contain this domain as well as to the 7tm_{—}1 domain itself.

TABLE 11
DOMAIN results for GPCR1
Sbjct: 7 transmembrane receptor (rhodopsin family) fragment (SEQ ID NO:29)
gnl|Pfam|pfam00001; Length = 377
Score = 125 bits (315), Expect = 3e-30

| | | |
|---|---|---|
| Query: 63 | GNGLSIYVFLQPYKKSTSVNVFMLNLAISDLLFISTLPFRADYYLRCSNWIFGDLACRIM | 122 |
| Sbjct: 1 | GNVLVCMAVSREKALQTTTNYLIVSLAVADLLVATLVMPWVVYLEVVGEWKFSRIHCDIF | 60 |
| | ** * : *: * :::**::*** : : * * * : * * | |
| Query: 123 | SYSLYVNMYSSIYFLTVLSVVRFLAMVHPFR-LLHVTSIRSAWILCGIIWILIMASSIML | 181 |
| Sbjct: 61 | VTLDVMMCTASILNLCAISIDRYTAVAMPMLYNTRYSSKRRVTVMIAIVWVLSFTISCPM | 120 |
| | : :** * :*: *: *: * :* * :: *:*:* * : | |
| Query: 182 | LDSGSEQNGSVTSCLELNLYKIAKLQTMNYIALVVGCLLPFFTLSICYLLIIRVLLKVEV | 241 |
| Sbjct: 121 | LFGLNNTDQN--ECIIA-------NPAFVVYSSIVSFYVPFIVTLLVYIKIYIVLRRRRK | 171 |
| | * : : : *: : :* :** : *: * ** : | |
| Query: 242 | PESGLR | 247 |
| Sbjct: 172 | RYNTER | 177 |
| | : * | |

Expression information for GPCRX RNA was derived using tissue sources including, but not limited to, proprietary database sources, public EST sources, literature sources, and/or RACE sources, as described in the Examples. GPCR1 is expressed in at least the following tissues: adrenal gland/suprarenal gland, heart, placenta, spleen, and peripheral blood leukocytes.

The nucleic acids and proteins of GPCR1 are useful in potential therapeutic applications implicated in various GPCR- or OR-related pathologies and/or disorders. For example, a cDNA encoding the G-protein coupled receptor-like protein may be useful in gene therapy, and the G-protein coupled receptor-like protein may be useful when administered to a subject in need thereof. The novel nucleic acid encoding GPCR1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. The GPCRX nucleic acids and proteins are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, fertility, hemophilia, hypercoagulation, idiopathic thrombocytopenic purpura, immunodeficiencies, graft versus host disease, bronchial asthma, and other diseases, disorders and conditions of the like. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Additional GPCR-related diseases and disorders are mentioned throughout the Specification.

Further, the protein similarity information, expression pattern, and map location for GPCR1 suggests that GPCR1 may have important structural and/or physiological functions characteristic of the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

These materials are further useful in the generation of antibodies that bind immunospecifically to the novel GPCR1 substances for use in therapeutic or diagnostic methods. These antibodies may be generated according to methods known in the art, using prediction from hydrophobicity charts, as described in the “Anti-GPCRX Antibodies” section below. In one embodiment, a contemplated GPCR1 epitope is from aa 30 to 60. In another embodiment, a GPCR1 epitope is from aa 80 to 95. In additional embodiments, GPCR1 epitopes are from aa 110 to 170, from aa 180 to 240; from aa 250 to 270, and from aa 280 to 305.

GPCR2

A second GPCR-like protein of the invention, referred to herein as GPCR2, is an Olfactory Receptor (“OR”)-like protein. Two alternative novel GPCR2 nucleic acids and encoded polypeptides are disclosed.

In one embodiment, a GPCR2a variant (alternatively referred to herein as AC022289_A) includes the 1039 nucleotide sequence (SEQ ID NO:3) shown in Table 2A. A GPCR2a ORF begins with an ATG initiation codon at nucleotides 54-56 and ends with a TGA codon at nucleotides 996-998. Putative untranslated regions upstream from the initiation codon and downstream from the termination codon are underlined in Table 2A, and the start and stop codons are in bold letters.

TABLE 2A
GPCR2a Nucleotide Sequence (SEQ ID NO:3).

ATATTTTGCTTTGGCAGGAACAATTCTCTTCAACCCTTCCATTAAAAGGAATTATGATGATGGTTTTAAGGAATCTGAGCATG
GAGCCCACCTTTGCCCTTTTAGGTTTCACAGATTACCCAAAGCTTCAGATTCCTCTCTTCCTTGTGTTTCTGCTCATGTATGT
TATCACAGTGGTAGGAAACCTTGGGATGATCATAATAATCAAGATTAACCCCAAATTTCACACTCCTATGTACTTTTTCCTTA
GTCACCTCTCTTTTGTTGATTTTTGTTACTCTTCCATTGTCACTCCCAAGCTGCTTGAGAACTTGGTAATGGCAGATAAAAGC
ATCTTCTACTTTAGCTGCATGATGCAGTACTTCCTGTCCTGCACTGCTGTGGTGACAGAGTCTTTCTTGCTGGCAGTGATGGC
CTATGACCGCTTTGTGGCCATCTGCAATCCTCTGCTTTATACAGTGGCCATGTCACAGAGGCTCTGTGCCCTGCTGGTGGCTG
GGTCATATCTCTGGGGCATGTTTGGCCCCTTGGTACTCCTTTGTTATGCTCTCCGGTTAAACTTCTCTGGACCTAATGTAATC
AACCACTTCTTTTGTGAGTATACTGCTCTCATCTCTGTGTCTGGCTCTGATATACTCATCCCCCACCTGCTGCTTTTCAGCTT
CGCCACCTTCAATGAGATGTGTACACTACTGATCATCCTCACTTCCTATGTTTTCATTTTTGTGACTGTACTAAAAATCCGTT
CTGTTAGTGGGCGCCACAAAGCCTTCTCCACCTGGGCCTCCCACCTGACTGCTATCACCATCTTCCATGGGACCATCCTTTTC
CTTTACTGTGTACCCAACTCCAAAAACTCTCGGCAAACAGTCAAAGTGGCCTCTGTATTTTACACAGTTGTCAACCCCATGCT
GAACCCTCCGATCTACAGCCTAAGGAATAAAGACGTGAAGGATGCTTTCTGGAAGTTAATACATACACAAGTTCCATTTCACT
GAACCAGTCTCAAAAGTTGTTTTCAATCCAAATGAACAACCCA

The GPCR2a polypeptide (SEQ ID NO:4) encoded by SEQ ID NO:3 is 314 aa and is presented using the one-letter amino acid code in Table 2B. The Psort profile for GPCR2 predicts that this sequence has a signal peptide and is likely to be localized at the plasma membrane with a certainty of 0.6000. The most likely cleavage site for a GPCR2a peptide is between amino acids 43 and 44, i.e., at the dash in the amino acid sequence VVG-NLG, based on the SignalP result.

TABLE 2B
GPCR2a protein sequence (SEQ ID NO:4)

MMMVLRNLSMEPTFALLGFTDYPKLQIPLFLVFLLMYVITVVGNLGMIIIIKINPKFHTPMYFFLSHLSFVDFCYSSIVTPKL
LENLVMADKSIFYFSCMMQYFLSCTAVVTESFLLAVAYDRFVAICNPLLYTVAMSQRLCALLVAGSYLWGMFGPLVLLLCYAL
RLNFSGPNVINHFFCEYTALISVSGSDILIPHLLLFSFATFNEMCTLLIILTSYVFIFVTVLKIRSVSGRHKAFSTWASHLTA
ITIFHGTILFLYCVPNSKNSRQTVKVASVFYTVPMLNPPIYSLRNKDVKDAFWKLIHTQVPFH

The predicted GPCR2a sequence, above, was subjected an the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then used as the forward and reverse primers in a PCR amplification based on a wide range of cDNA libraries. The resulting amplicon was gel purified, cloned and sequenced to high redundancy, as described in the Examples.

The cloned sequence is disclosed as an alternative embodiment of GPCR2 (SEQ ID NO:5), referred to herein as the GPCR2b and reported in Tables 2C and 2D. GPCR2b is alternatively referred to herein as AC022289_A1. The GPCR2b ORF begins with an ATG initiation codon at nucleotides 54-56 and ends with a TGA codon at nucleotides 996-998. Putative untranslated regions upstream from the initiation codon and downstream from the termination codon are underlined in Table 2C, and the start and stop codons are in bold letters.

TABLE 2C
GPCR2b Nucleotide Sequence

ATATTTTGCTTTGGCAGGAACAATTCTCTTCAACCCTTCCATTAAAAGGAATTATGATGATGGTTTTAAGGAATCTGAGCATG
GAGCCCACCTTTGCCCTTTTAGGTTTCACAGATTACCCAAAGCTTCAGATTCCTCTCTTCCTTGTGTTTCTGCTCATGTATGT
TATCACAGTGGTAGGAAACCTTGGGATGATCATAATAATCAAGATTAACCCCAAATTTCACACTCCTATGTACTTTTTCCTTA
GTCACCTCTCTTTTGTTGATTTTTGTTACTCTTCCATTGTCACTCCCAAGCTGCTTGAGAACTTGGTAATGGCAGATAAAAGC
ATCTTCTACTTTAGCTGCATGATGCAGTACTTCCTGTCCTGCACTGCTGTGGTGACAGAGTCTTTCTTGCTGGCAGTGATGGC
CTATGACCGCTTTGTGGCCATCTGCAATCCTCTGCTTTATACAGTGGCCATGTCACAGAGGCTCTGTGCCCTGCTGGTGGCTG
GGTCATATCTCTGGGGCATGTTTGGCCCCTTGGTACTCCTTTGTTATGCTCTCCGGTTAAACTTCTCTGGACCTAATGTAATC
AACCACTTCTTTTGTGAGTATACTGCTCTCATCTCTGTGTCTGGCTCTGATATACTCATCCCCCACTTGCTGCTTTTCAGCTT
CGCCACCTTCAATGAGATGTGTACACTACTGATCATCCTCACTTCCTATGTTTTCATTTTTGTGACTGTACTAAAAATCCGTT
CTGTTAGTGGGCGCCACAAAGCCTTCTCCACCTGGGCCTCCCACCTGACTGCTATCACCATCTTCCATGGGACCATCCTTTTC
CTTTACTGTGTACCCAACTCCAAAAACTCTCGGCAAACAGTCAAAGTGGCCTCTGTATTTTACACAGTTGTCAACCCCATGCT
GAACCCTCTGATCTACAGCCTAAGGAATAAAGACGTGAAGGATGCTTTCTGGAAGTTAATACATACACAAGTTCCATTTCACT
GAACCAGTCTCAAAAG

The GPCR2b protein (SEQ ID NO:6) encoded by SEQ ID NO:5 is 314 amino acid in length, has a molecular weight of 35806.5 Daltons, and is presented using the one-letter code in Table 2D. As with GPCR2a, the most likely cleavage site for a GPCR2b peptide is between amino acids 43 and 44, i.e., at the dash in the amino acid sequence VVG-NLG, based on the SignalP result.

TABLE 2D
GPCR2b protein sequence

MMMVLRNLSMEPTFALLGFTDYPKLQIPLFLVFLLMYVITVVGNLGMIIIIKINPKFHTPMYFFLSHLSFVDFCYSSIVTPKL
LENLVMADKSIFYFSCMMQYFLSCTAVVTESFLLAVMAYDRFVAICNPLLYTVAMSQRLCALLVAGSYLWGMFGPLVLLCYAL
RLNFSGPNVINHFFCEYTALISVSGSDILIPHLLLFSFATFNEMCTLLIILTSYVFIFVTVLKIRSVSGRHKAFSTWASHLTA
ITIFHGTILFLYCVPNSKNSRQTVKVASVFYTVVNPMLPLIYSLRNKDVKDAFWKLIHTQVPFH

Unless specifically addressed as GPCR2a or GPCR2b, any reference to GPCR2 is assumed to encompass all variants. Residue differences between any GPCRX variant sequences herein are written to show the residue in the “a” variant, the residue position with respect to the “a” variant, and the residue in the “b” variant. GPCRX residues in all following sequence alignments that differ between the individual GPCRX variants are highlighted in black and marked with the (o) symbol above the variant residue in all alignments herein. For example, the GPCR2 nucleic acid sequences differ at the following two positions: C648T and C922T. The GPCR2 polypeptides differ only at one residue, namely P290L.

In a BLASTN search of sequence databases, it was found, for example, that the GPCR2a nucleic acid sequence has 471 of 648 bases (72%) identical to Rattus norvegicus taste bud receptor protein (SEQ ID NO:30), as shown in Table 2E. The BLASTN alignment shown in Table 2E result from a search utilizing the nucleotide sequence for GPCR2a. The residue that differs between GPCR2a and GPCR2b is highlighted in black and marked with the (o) symbol.

TABLE 2E
BLASTN of GPCR2 against rat taste bud receptor protein.

>gb:GENBANK-ID:RNU50948|acc:U50948 Rattus norvegicus taste bud receptor protein TB
567 (TB 567) gene, complete cds - Rattus norvegicus, 1299 bp. (SEQ ID NO:30)
Score = 1221 (183.2 bits), Expect = 3.3e-49, P = 3.3e-49
Identities = 591/940 (62%), Positives = 591/940 (62%), Strand = Plus / Plus

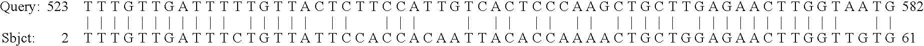

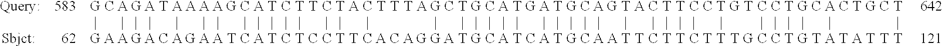

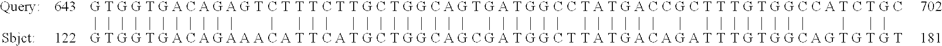

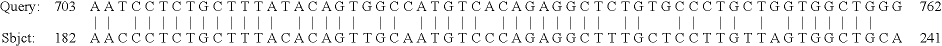

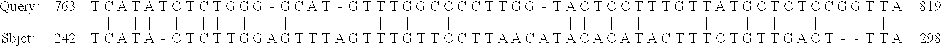

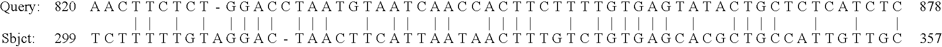

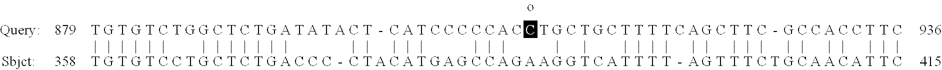

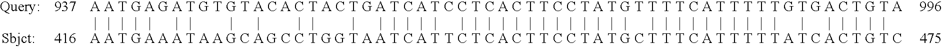

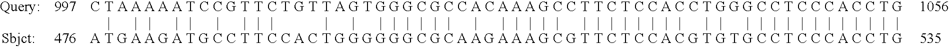

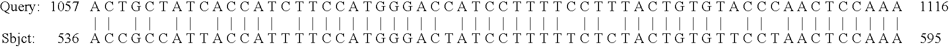

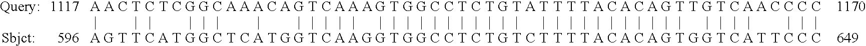

The GPCR2 nucleic acid sequence has homology to two regions of the Homo sapiens olfactory receptor (“OR5D3”) gene, as shown in Table 2F. OR5D3 residues 437-644 (SEQ ID NO:31) has 168 of 208 bases (80%) identical to GPCR2, with an E value of 4×10⁻¹⁷. OR5D3 residues 121-219 (SEQ ID NO:32) has 82 of 99 bases (82%) identical to GPCR2, with an E value of 6×10⁻⁷.

TABLE 2F
BLASTN of GPCR2 against the OR5D3 gene

gb |AF065860.1|AF065860 Homo sapiens olfactory receptor (0R5D3) gene,
partial cds
Length = 649; Score = 95.6 bits (48), Expect = 4e-17;
Identities = 168/208 (80%); Strand = Plus/Plus

| | | |
|---|---|---|
| Query: 696 | atcatcctcacttcctatgttttcatttttgtgactgtactaaaaatccgttctgttagt | 755 |
| | \|\|\|\|\| \|\|\|\|\|\|\|\|\|\|\|\|\| \|\|\|\|\|\|\|\|\|\| \| \|\|\|\|\| \| \|\| \|\| \| \|\|\| \| \| | |
| Sbjct: 437 | atcattctcacttcctatgctttcatttttatcactgtcatgaagatgccttccactggg | 496 |
| Query: 756 | gggcgccacaaagccttctccacctgggcctcccacctgactgctatcaccatcttccat | 815 |
| | \|\|\|\|\|\| \| \|\|\|\|\| \|\|\|\|\|\|\|\| \|\| \|\|\|\|\|\|\|\|\|\|\|\|\|\| \|\| \|\| \|\|\|\|\| \|\|\|\|\|\| | |
| Sbjct: 497 | gggcgcaagaaagcgttctccacgtgtgcctcccacctgaccgccattaccattttccat | 556 |
| Query: 816 | gggaccatccttttcctttactgtgtacccaactccaaaaactctcggcaaacagtcaaa | 875 |
| | \|\|\|\|\| \|\|\|\|\|\|\|\| \|\| \|\|\|\|\|\|\|\| \|\| \|\|\|\|\|\|\|\|\|\| \|\| \|\|\| \| \|\|\|\|\| | |
| Sbjct: 557 | gggactatcctttttctctactgtgttcctaactccaaaagttcatggctcatggtcaag | 616 |
| Query: 876 | gtggcctctgtattttacacagttgtca | 903 |
| | \|\|\|\|\|\|\|\|\|\|\| \|\|\|\|\|\|\|\|\|\|\| \|\|\|\| | |
| Sbjct: 617 | gtggcctctgtcttttacacagtggtca | 644 (SEQ ID NO:31) |

Score = 61.9 bits (31), Expect = 6e-07
Identities = 82/99 (82%) Strand = Plus/Plus

| | | |
|---|---|---|
| Query: 380 | tgtggtgacagagtctttcttgctggcagtgatggcctatgaccgctttgtggccatctg | 439 |
| | \|\|\|\|\|\|\|\|\|\|\|\| \| \|\|\| \|\|\|\|\|\|\|\|\| \|\|\|\|\|\| \|\|\|\|\|\| \| \|\|\|\|\|\|\|\| \| \|\| | |
| Sbjct: 121 | tgtggtgacagaaacattcatgctggcagcgatggcttatgacagatttgtggcagtgtg | 180 |
| Query: 440 | caatcctctgctttatacagtggccatgtcacagaggct | 478 |
| | \|\| \|\|\|\|\|\|\|\|\|\|\| \|\|\|\|\| \|\| \|\|\|\|\| \|\|\|\|\|\|\|\| | |
| Sbjct: 181 | taaccctctgctttacacagttgcaatgtcccagaggct | 219 (SEQ ID NO:32) |

The full GPCR2 amino acid sequence has 159 of 306 amino acid residues (51%) identical to, and 214 of 306 residues (69%) positive with, the 314 amino acid residue proteins from Homo sapiens Olfactory Receptor-like protein OLF1 (ptnr: SPTREMBL-ACC: Q13606) (SEQ ID NO:33) (Table 2G). The residue that differs between GPCR2a and GPCR2b is highlighted in black and marked with the (o) symbol.

TABLE 2G
BLASTX of GPCR2 against OR-like Protein OLF1

>ptnr:SWISSPROT-ACC:Q13606 OLFACTORY RECEPTOR-LIKE PROTEIN OLF1 - Homo sapiens
(Human), 314 aa. (SEQ ID NO:33)
Score = 814 (286.5 bits), Expect = 1.6e-80, P = 1.6e-80
Identities = 159/306 (51%), Positives = 214/306 (69%), Frame = +1

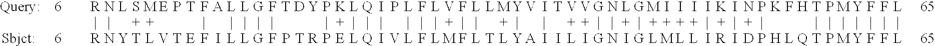

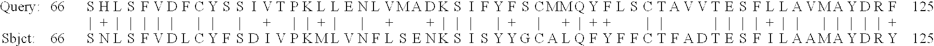

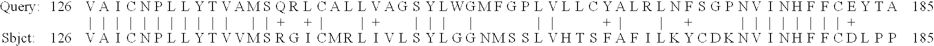

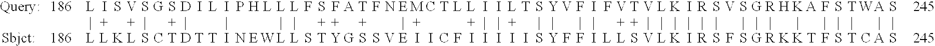

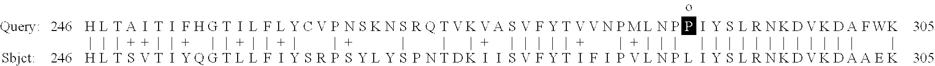

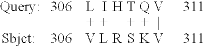

The full amino acid sequence of the GPCR2 protein of the invention has 152 of 301 amino acid residues (50%) identical to, and 207 of 301 residues (68%) positive with, the 312 amino acid residue proteins from Gallus gallus olfactory receptor 4 (ptnr: SPTREMBL-ACC: CAA64370.1) (SEQ ID NO:34) (Table 2H). The residue that differs between GPCR2a and GPCR2b is highlighted in black and marked with the (o) symbol.

TABLE 2H
BLASTX of GPCR2 against OR-4

>emb|CAA64370.1| (X94744) olfactory receptor 4 [Gallus gallus]; (SEQ ID NO:34), 312 aa
Statistics for GPCR2a: Score = 304 bits (779), Expect = 8e-82
Identities = 152/301 (50%), Positives = 207/301 (68%)
Statistics for GPCR2b: Score = 320 bits (821), Expect = 1e-86
Identities = 160/306 (52%), Positives = 215/306 (69%)

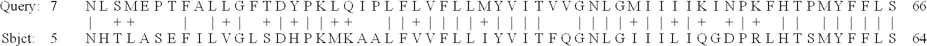

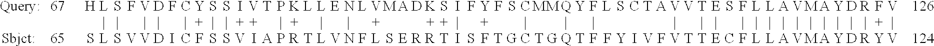

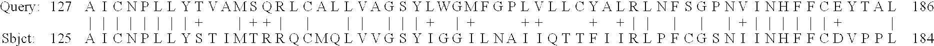

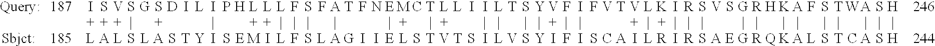

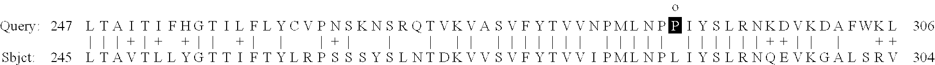

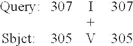

A multiple sequence alignment is given in Table 21, with the GPCR2 protein of the invention being shown on line 1, in a ClustalW analysis comparing GPCR2 with related protein sequences.

TABLE 2I
Information for the ClustalW proteins:

1. Novel_Human_OLF, i.e. GPCR2, SEQ ID NO:4
2. Homo sapiens OLF, ptnr-SWISSPROT Acc # Q13606, SEQ ID NO:33
3. Gallus gallus OLF, ptnr-SWISSPROT Acc # P37070, SEQ ID NO:35
4. Rattus norvegicus OLF, Acc # Q63395, SEQ ID NO:36

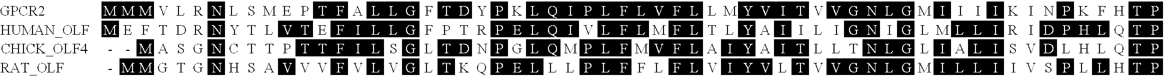

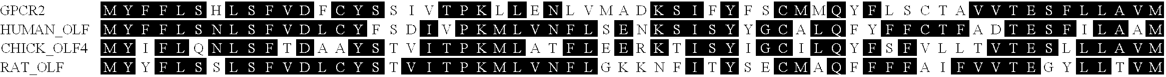

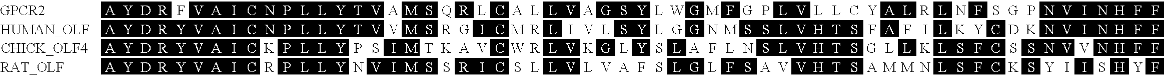

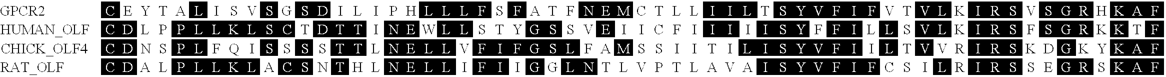

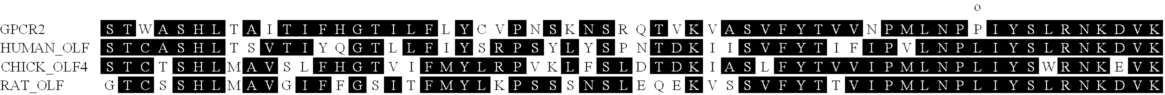

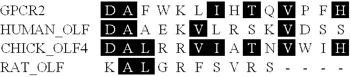

Domain results for GPCR2 were collected from the Conserved Domain Database (CDD) with Reverse Position Specific BLAST. This BLAST samples domains found in the Smart and Pfam collections. The results are listed in Table 2J with the statistics and domain description. The 7tm_{—}1, a seven transmembrane receptor (rhodopsin family), was shown to have two segments with significant homology to GPCR2. An alignment of GPCR2 with residues 1-170 (SEQ ID NO:29) and residues 310-377 (SEQ ID NO:37) of 7tm_{—}1 are shown in Table 2J.

TABLE 2J
DOMAIN results for GPCR2

gnl|Pfam|pfam00001, 7tm_1, 7 transmembrane receptor (rhodopsin family) (SEQ ID NO:29)
Length = 377; Score = 83.2 bits (204), Expect = 2e-17

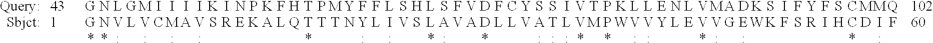

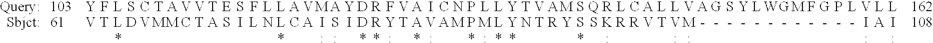

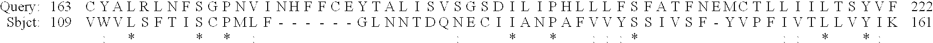

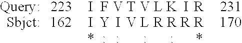

gnl|Pfam|pfam00001, 7tm_1, 7 transmembrane receptor (rhodopsin family) (SEQ ID NO:37)
Length = 377 Score = 35.8 bits (81), Expect = 0.003

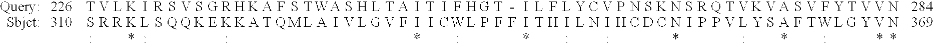

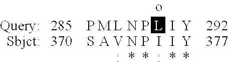

The nucleic acids and proteins of GPCR2 are useful in potential therapeutic applications implicated in various GPCR-related pathological disorders and/or OR-related pathological disorders, described further below. For example, a cDNA encoding the olfactory receptor-like protein may be useful in gene therapy, and the olfactory receptor-like protein may be useful when administered to a subject in need thereof. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Other GPCR-1 diseases and disorders are contemplated.

The novel nucleic acid encoding GPCR2, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. This novel protein also has immense value in development of powerful assay system for functional analysis.

GPCR3

An additional GPCR-like protein of the invention, referred to herein as GPCR3, is an Olfactory Receptor (“OR”)-like protein. The GPCR3 nucleic acid of 1001 nucleotides (also designated AP001112_A) is shown in Table 3A. An ORF was identified beginning with an ATG initiation codon at nucleotides 12-14 and ending with a TAA codon at nucleotides 945-47. A putative untranslated region upstream from the initiation codon and downstream from the termination codon is underlined in Table 3A, and the start and stop codons are in bold letters.

TABLE 3A
GPCR3 Nucleotide Sequence (SEQ ID NO:7)

CGAAGGAAATTATGAGAAGAAACTGCACGTTGGTGACTGAGTTCATTCTCCTGGGACTGACCAGTCGCCGGGAATTACAAATT
CTCCTCTTCACGCTGTTTCTGGCCATTTACATGGTCACGGTGGCAGGGAACCTTGGCATGATTGTCCTCATCCAGGCCAACGC
CTGGCTCCACATGCCCATGTACTTTTTCCTGAGCCACTTATCCTTCGTGGATCTGTGCTTCTCTTCCAATGTGACTCCAAAGA
TGCTGGAGATTTTCCTTTCAGAAAAGAAAAGCATTTCCTATCCTGCCTGTCTTGTGCAGTGTTACCTTTTTATCGCCTTGGTC
CATGTTGAGATCTACATCCTGGCTGTGATGGCCTTTGACCGGTACATGGCCATCTGCAACCCTCTGCTTTATGGCAGCAGAAT
GTCCAAGAGTGTGTGCTCCTTCCTCATCACGGTGCCTTATGTGTATGGAGCGCTCACTGGCCTGATGGAGACCATGTGGACCT
ACAACCTAGCCTTCTGTGGCCCCAATGAAATTAATCACTTCTACTGTGCGGACCCACCACTGATTAAGCTGGCTTGTTCTGAC
ACCTACAACAAGGAGTTGTCAATGTTTATTGTGGCTGGCTGGAACCTTTCTTTTTCTCTCTTCATCATATGTATTTCCTACCT
TTACATTTTCCCTGCTATTTTAAAGATTCGCTCTACAGAGGGCAGGCAAAAAGCTTTTTCTACCTGTGGCTCCCATCTGACAG
CTGTCACTATATTCTATGCAACCCTTTTCTTCATGTATCTCAGACCCCCCTCAAAGGAATCTGTTGAACAGGGTAAAATGGTA
GCTGTATTTTATACCACAGTAATCCCTATGCTGAACCTTATAATTTATAGCCTTAGAAATAAAAATGTAAAAGAAGCATTAAT
CAAAGAGCTGTCAATGAAGATATACTTTTCTTAAAAATCAGTATTCTTTTGGTTTCTAAAGCCCTTCCTAGACTTTTTTCTTT
AGCTG

The GPCR3 polypeptide (SEQ ID NO:8) encoded by SEQ ID NO:7 is 311 amnino acid residues and is presented using the one-letter code in Table 3B.

TABLE 3B
Encoded GPCR3 protein sequence (SEQ ID NO:8).

MRRNCTLVTEFILLGLTSRRELQILLFTLFLAIYMVTVAGNLGMIVLIQANAWLHMPMYFFLSHLSFVDLCFSSNVTPKMLEI
FLSEKKSISYPACLVQCYLFIALVHVEIYILAVMAFDRYMAICNPLLYGSRNSKSVCSFLITVPYVYGALTGLMETMWTYNLA
FCGPNEINHFYCADPPLIKLACSDTYNKELSMFIVAGNNLSFSLFIICISYLYIFPAILKIRSTEGRQKAFSTCGSHLTAVTI
FYATLFFMYLRPPSKESVEQGKMVAVFYTTVIPMLNLIIYSLRNKNVKEALIKELSMKIYFS

In a search of sequence databases, it was found, for example, that the GPCR3 nucleic acid sequence has 609 of 923 bases (65%) identical to a and 609/923 bases (65%) positive with Pan troglodytes species Olfactory Receptor OR93 gene (SEQ ID NO:38), as shown in Table 3C.

TABLE 3C
BLASTN of GPCR3 against Chimpanzee OR93 gene

>gb:GENBANK-ID:AF045577|acc:AF045577 Pan troglodytes olfactory receptor
OR93Ch
(OR93) gene, complete cds - Pan troglodytes, (SEQ ID NO:38), 989 bp
Score = 1405 (210.8 bits), Expect = 2.1e-57, P = 2.1e-57
Identities = 609/923 (65%), Positives = 609/923 (65%), Strand = Plus/Plus

| | | |
|---|---|---|
| Query: | 20 AAACTGCACGTTGGTGACTGAGTTCATTCTCCTGGGACTGACC-AGTCGCCGGGAATTAC | 78 |
| | \|\|\|\|\| \|\|\| \|\|\| \|\| \|\| \|\|\|\|\|\| \|\| \|\| \|\|\| \| \|\| \| \|\| \| | |
| Sbjct: | 12 AAACTACACAAAGGTCACCGAATTCATTTTCACAGGCTTGAATTACAATCCTC-AGTTGC | 70 |
| Query: | 79 AAATTCTCCTCTTCACGCTGTTTCTGGCCATTTAC-ATGGTCA-CGGTGGCAG-GGAACC | 135 |
| | \| \| \|\|\|\|\|\|\|\| \|\| \|\|\|\|\|\| \| \| \|\| \| \|\|\| \|\|\| \| \|\| \| \|\|\|\| \| | |
| Sbjct: | 71 AGGTCTTCCTCTTCCTACTCTTTCTGACAACTTTCTATG-TCATCAATGTAACTGGAAAC | 129 |
| Query: | 136 TTGGCA-TGATTGTCCTCATCCAGGCCAACGCCTGGCTCCACATGCCCATGTACTTTTTC | 194 |
| | \|\|\|\| \| \|\|\|\|\|\|\|\|\|\| \|\|\|\| \| \| \|\| \| \|\| \|\|\|\| \|\|\|\|\|\|\|\|\|\|\|\|\|\|\| | |
| Sbjct: | 130 TTGGGAATGATTGTCCTTATCCGAATCGATTCCCGCCTTCACACACCCATGTACTTTTTC | 189 |
| Query: | 195 CTGAGCCACTTATCCTTCGTGGATCTGTGCTTCTCTTCCAATGTGACTCCAAAGATGCTG | 254 |
| | \|\| \|\|\|\|\|\| \| \|\|\|\|\| \|\|\|\|\| \| \|\|\|\|\|\|\|\| \|\| \|\|\|\|\| \|\| \|\|\|\|\|\|\|\|\| | |
| Sbjct: | 190 CTCAGCCACCTGTCCTTTGTGGACATCTGCTTCTCCTCAGTTGTGAGCCCCAAGATGCTC | 249 |
| Query: | 255 GA-GATTTTCCTTTCAGAGAAGAAAAGC-ATTTCCTATCCT-GCCTGT-CTT-GT-GCAG | 308 |
| | \|\| \|\| \|\|\|\| \|\| \|\| \|\|\|\|\| \|\|\|\|\| \| \|\|\|\| \| \|\|\|\| \|\|\| \| \|\|\|\| | |
| Sbjct: | 250 ACTGACTT--CTTTGTGAAGAGGAAAGCCATTTCTT-TCCTTGGCTGTGCTTTGCAGCAG | 306 |
| Query: | 309 TGTTACCTTTTTATCGCCTTGGTCCATGTTGAGATCTACATCCTGGCTGTG-ATGGCCTT | 367 |
| | \|\| \| \| \|\|\| \| \| \|\|\| \|\| \| \|\|\| \| \| \|\| \|\|\|\| \|\| \|\|\|\|\|\|\| | |
| Sbjct: | 307 TGGTTC-TTTGGGTT-CTTTGTGGCA-GCAGAGTGTTTCCTCTTGGC-GTCCATGGCCTA | 362 |
| Query: | 368 TGACCGGTACATGGCCATCTGCAACCCTCTGCTTTATGGCAGCAGA-ATGTCCAAGAGTG | 426 |
| | \|\|\|\|\|\| \|\| \|\|\|\|\|\|\|\|\|\| \|\|\|\|\| \|\| \| \|\| \|\|\| \| \|\|\|\|\|\| \|\|\|\| | |
| Sbjct: | 363 TGACCGCTATGTGGCCATCTGTAACCCATTGTTATACT-CAGTTGCTATGTCCCAGAGGC | 421 |
| Query: | 427 TGTGC-TCCTTCCTCATCACGGTGCCTTATGTGTATGGAGCGC-TCACTGGCCTGATGGA | 484 |
| | \| \|\|\| \|\|\| \| \| \| \|\| \|\| \|\|\|\|\| \|\|\|\| \| \| \| \| \| \| \|\|\| \| | |
| Sbjct: | 422 TCTGCATCCAGC-TAGTGGTGGGTCCCTATGTCATTGGA-CTCATGAATACCATGACTCA | 479 |
| Query: | 485 GAC--CATGTGGACCTACAACCTAGCCTTCTGTGGCCCCAATGAAATTAATCACTTCTAC | 542 |
| | \|\| \|\| \|\| \| \| \| \| \| \|\| \|\|\|\|\|\|\|\|\|\| \|\|\|\| \|\| \|\|\|\|\| \|\|\|\| \| | |
| Sbjct: | 480 CACAACAAATGCATTT-CGTC-TCCCTTTTTGTGGCCCTAATGTCATCAATCATTTCTTC | 537 |
| Query: | 543 TGTGCGGACCCACCACTGA-TTAAGCTGGCTTGTTCTGACACCTACAACAAGGAGTTGTC | 601 |
| | \|\|\|\| \| \|\| \|\| \| \|\| \|\| \| \|\|\| \|\|\|\| \|\|\| \|\|\| \|\|\|\|\| \| | |
| Sbjct: | 538 TGTGATATGTCCCCC-TTACTTTCCCTTGTATGTGCTGATACCAGGCTCAATAAGTTGGC | 596 |
| Query: | 602 AATGTTTATTGTGGCTGG--CTG-GAACCT-TTCTTTT-TCTCTCTTCATCATATGTATT | 656 |
| | \| \| \|\| \|\| \|\|\|\|\|\|\|\| \|\|\| \| \| \|\|\| \| \|\|\| \|\| \|\|\| \| \| \|\|\| | |
| Sbjct: | 597 AGTTTTCATCGTGGCTGGAGCTGCGGGAGTCTTCAGTGGTCTGACT--ATCCT--G-ATT | 651 |
| Query: | 657 TCCTACCTTTACATTTTCCCTGCTATTTTAAAGATTCGCTCTACAGAGGGCAGGCAAAAA | 716 |
| | \|\|\|\|\|\| \|\|\|\|\|\|\| \|\| \|\| \|\| \| \| \|\|\| \|\|\|\|\|\| \| \|\| \|\| \|\|\| \|\|\| | |
| Sbjct: | 652 TCCTACATTTACATCCTCATGGCCATCCTGAGGATCCGCTCTGCTGATGGGAGGTGCAAA | 711 |
| Query: | 717 GCTTTTTCTACCTGTGGCTCCCATCTGACAGCTGTCACTATATTCTATGCAACCCTTTTC | 776 |
| | \| \|\|\|\|\|\|\|\| \|\| \|\| \|\| \|\|\|\|\|\|\|\|\|\|\| \|\| \|\| \|\|\|\| \|\|\|\|\|\|\|\|\| | |
| Sbjct: | 712 ACCTTTTCTACTTGCTCTTCTCACCTGACAGCTGTTTTCATCTTGTATGGTACCCTTTTC | 771 |
| Query: | 777 TTCATGTATCTCAGACCCCCCTCAAAGGAATCTGTTGAACAGGGTAAAATGGTAGCTGTA | 836 |
| | \|\| \|\| \|\|\| \| \| \|\| \|\|\| \|\| \| \|\| \| \|\|\|\| \| \|\| \|\|\|\| | |
| Sbjct: | 772 TTTATTTATGTACGTCCTAGTGCAAGCTTCTCCCTGGATCTCAATAAATTAGTGTCTGTG | 831 |
| Query: | 837 TTTTATACCACAGTAATCCCTATGCTGAACCTTATAATTTATAGCCTTAGAAATAAAAAT | 896 |
| | \|\|\|\|\| \|\| \|\|\|\| \|\| \|\|\|\|\|\| \|\|\|\|\|\| \| \|\| \|\| \|\|\| \| \|\|\|\|\| \|\| \| | |
| Sbjct: | 832 TTTTACACAGCAGTGATTCCTATGTTGAACCCACTTATCTACAGCTTGAGAAACAAGGAA | 891 |
| Query: | 897 GTAAAAGAAGC-ATTAATCAAAGAGCTGTCAATGAAGATATACTTTT | 942 |
| | \|\| \|\|\|\|\| \|\| \|\| \| \|\| \|\| \|\|\|\|\|\| \| \| \|\| \| \|\|\|\| | |
| Sbjct: | 892 GTCAAAGATGCCATCCA-CAG-GA-CTGTCACTCA-GAGGAAGTTTT | 934 |

The BLASTN alignment shown in Table 3D indicates that two fragments of GPCR3 have homology to fragments of Mus musculus olfactory receptor 4 cluster, gene 3 (“Olfr4-3”) (GENBANK-ID: NM_{—}013728.1). Residues 827-907 (SEQ ID NO:39) and residues 163-210 (SEQ ID NO:40) of the Olfr4-3 gene are shown below.

TABLE 3D
BLASTN of GPCR3 against Olfr4-3

>ref|NM_013728.1| Mus musculus olfactory receptor
4 cluster, gene 3 (Olfr4-3), mRNA
Length = 957 (SEQ ID NO:39)
Score = 58.0 bits (29), Expect = 9e-06
Identities = 68/81 (83%)
Strand = Plus/Plus

| | | |
|---|---|---|
| Query: | 835 tattttataccacagtaatccctatgctgaaccttataatttatagccttagaaataaaa | 894 |
| | \|\|\|\|\|\|\|\|\|\|\|\|\| \|\| \|\|\|\|\|\|\|\|\| \|\|\|\|\|\| \| \|\|\|\|\|\|\|\|\|\|\| \|\|\|\|\|\|\|\| | |
| Sbjct: | 827 tattttataccactgttatccctatgttgaacccattcatttatagcctgagaaataagg | 886 |
| Query: | 895 atgtaaaagaagcattaatca | 915 |
| | \| \|\| \|\|\|\|\| \|\|\|\|\|\|\|\|\|\| | |
| Sbjct: | 887 aagtcaaagatgcattaatca | 907 |

>ref|NM_013728.1| Mus musculus olfactory receptor
4 cluster, gene 3 (Olfr4-3), mRNA
Length = 957 (SEQ ID NO:40)
Score = 56.0 bits (28), Expect = 4e-05
Identities = 43/48 (89%)
Strand = Plus/Plus

| | | |
|---|---|---|
| Query: | 171 ctccacatgcccatgtactttttcctgagccacttatccttcgtggat | 218 |
| | \|\|\|\|\|\|\|\| \|\|\|\|\|\|\|\|\|\|\|\|\|\| \|\| \|\|\|\|\|\|\|\| \|\|\|\|\| \|\|\|\|\|\| | |
| Sbjct: | 163 ctccacatccccatgtacttttttctcagccacttgtcctttgtggat | 210 |

The full GPCR3 amino acid sequence has 166 of 305 amino acid residues (54%) identical to, and 214 of 305 residues (70%) positive with, the 314 amino acid residue OR93CH protein from Pan troglodytes (ptnr: SPTREMBL-ACC: 077756) (SEQ ID NO:41) (Table 3E).

TABLE 3E
BLASTX of GPCR3 against OR93CH-(SEQ ID NO:41)

>SPTREMBL-ACC:077756 OLFACTORY RECEPTOR OR93CH - Pan troglodytes
(Chimpanzee), 314 aa.
Score = 832 (292.9 bits), Expect = 3.6e-82, P = 3.6e-82
Identities = 166/305 (54%), Positives = 214/305 (70%), Frame = +3

| | | |
|---|---|---|
| Query: 4 | NCTLVTEFILLGLTSRRELQILLFTLFLA-IYMVTVAGNLGMIVLIQANAWLHMPMYFFL | 62 |
| | \| \| \|\|\|\|\| \|\| +\|\|+ \|\| \|\|\| \|++ \| \|\|\|\|\|\|\|\|\|+ ++ \|\| \|\|\|\|\|\| | |
| Sbjct: 5 | NYTKVTEFIFTGLNYNPQLQVFLFLLFLTTFYVINVTGNLGMIVLIRIDSRLHTPMYFFL | 64 |
| Query: 63 | SHLSFVDLCFSSNVTPKMLEIFLSEKKSISYPACLVQCYLFIALVHVEIYILAVMAFDRY | 122 |
| | \|\|\|\|\|\|\|+\|\|\|\| \|+\|\|\|\| \| ++\|+\|\|+ \| +\| +\| \| \| ++\|\| \|\|+\|\|\| | |
| Sbjct: 65 | SHLSFVDICFSSVVSPKMLTDFFVKRKAISFLGCALQQWFFGFFVAAECFLLASMAYDRY | 124 |
| Query: 123 | MAICNPLLYGSRMSKSVCSFLITVPYVYGALTGLMETMWTYNLAFCGPNEINHFYCADPP | 182 |
| | +\|\|\|\|\|\|\|\| \|\|+ +\| \|+ \|\|\| \| + + \| + \| \|\|\|\|\| \|\|\|\|+\| \| | |
| Sbjct: 125 | VAICNPLLYSVAMSQRLCIQLVVGPVVIGLMNTMTHTTNAFRLPFCGPNVINHFFCDMSP | 184 |
| Query: 183 | LIKLACSDTYNKELSMFIVAGWNLSFSLFIICISYLYIFPAILKIRSTEGRQKAFSTCGS | 242 |
| | \|+ \| \|+\|\| +\|++\|\|\|\|\| \|\| \| \|\|\|+\|\| \|\|\|+\|\|\| +\|\| \| \|\|\|\| \| | |
| Sbjct: 185 | LLSLVCADTRLNKLAVFIVAGAAGVFSGLTILISYIYILMAILRIRSADGRCKTFSTCSS | 244 |
| Query: 243 | HLTAVTIFYATLFFMYLRPPSKESVEQGKMVAVFYTTVIPMLNLIIYSLRNKNVKEALIK | 302 |
| | \|\|\|\|\| \| \| \|\|\|\|+\|+\|\| + \|++ \|+\|+\|\|\|\| \|\|\|\|\|\| +\|\|\|\|\|\|\| \|\|+\|+ \| | |
| Sbjct: 245 | HLTAVFILYGTLFFIYVRFSASFSLDLNKLVSVFYTAVIPMLNPLIYSLRNKEVKDAIHR | 304 |
| Query: 303 | ELSMK | 307 |
| | ++ + | |
| Sbjct: 305 | TVTQR | 309 |

The full amino acid sequence of the GPCR3 protein was also found to have 166 of 311 amino acid residues (53%) identical to, and 215 of 311 residues (68%) positive with, the 311 amino acid residue proteins from Mus musculus olfactory receptor 4 cluster, gene 3 (SEQ ID NO:42), shown in Table 3F.

TABLE 3F
BLASTX of GPCR3 against mouse OR4 cluster, gene 3

>ref|NP_038756.1| olfactory receptor 4 cluster, gene 3
[Mus musculus], derived from
gb|AAF20365.1|AF146372_1 (AF146372) olfactory receptor OR912-93
[Mus musculus domesticus] (SEQ ID NO:42).
Length = 318; Score = 321 bits (823), Expect = 6e-87
Identities = 166/311 (53%), Positives = 215/311 (68%)

| | | |
|---|---|---|
| Query: 1 | MRRNCTLVTEFILLGLTSRRELQILLFTLFLAIYMVTVAGNLGMIVLIQANAWLHNPMYF | 60 |
| | \| \|\| \|+\|\|\|\| \|\|\|\| \|\|\|+\|\| \|\| +\|+ \|+ \|\|\|\|\|\|+\|\| + \|\|+\|\|\|\| | |
| Sbjct: 2 | MHRNQTVVTEFFFTGLTSSFELQIVLFLTFLCVYLATLLGNLGMIILIHLDTRLHIPMYF | 61 |
| Query: 61 | FLSHLSFVDLCFSSNVTPKMLEIFLSEKKSISYPACLVQCYLFIALVHVEIYILAVMAFD | 120 |
| | \|\|\|\|\|\|\|\|\| \| \|\| ++\|\|\|\| +\|\| \|\|+ \| +\| \|\| \| \| ++\|\| \|\|+\| | |
| Sbjct: 62 | FLSHLSFVDACSSSVISPKMLSDMFVDKKVISFLGCAIQLCLFSQFVVTECFLLASMAYD | 121 |
| Query: 121 | RYMAICNPLLYGSRMSKSVCSFLITVPYVYGALTGLMETMWTYNLAFCGPNEINHFYCAD | 180 |
| | \|\|+\|\|\| \|\|\|\| \|\|+ \|\| \|+ \|\| \| ++ ++ + + \| +\|\|\|\| \|\|\|\|+\| | |
| Sbjct: 122 | RYVAICKPLLYTLIMSQRVCVQLVIGPYSIGFISTMVHIISAFVLPYCGPNLINHFFCDL | 181 |
| Query: 181 | PPLIKLACSDTYNKELSMFIVAGWNLSFSLFIICISYLYIFPAILKIRSTEGRQKAFSTC | 240 |
| | \|++ \|\|\|++\| + +\|\|\|\|\| \|\| \|\| +\|\|+\|\| \|\|\|\| \| +\|\|+\|\|\|\|\|\| | |
| Sbjct: 182 | LPVLSLACANTQMNERLLFIVAGILGVFSGIIILVSYVYIAITILKISSADGRRKAFSTC | 241 |
| Query: 241 | GSHLTAVTIFYATLFFMYLRPPSKESVEQGKMVAVFYTTVIPMLNLIIYSLRNKNVKEAL | 300 |
| | \|\|\|\|\|\|+\| \| \|\|\|\|+\|+\|\| \| \|++ \|+\|++\|\|\|\|\|\|\|\|\|\| \|\|\|\|\|\|\| \|\|+\|\| | |
| Sbjct: 242 | SSHLTAVSILYGTLFFIYVRPSSSFSLDINKVVSLFYTTVIPMLNPFIYSLRNKEVKDAL | 301 |
| Query: 301 | IKELSMKIYFS | 311 |
| | \|+ + +\| | |
| Sbjct: 302 | IRTFEKQFCYS | 312 |

A multiple sequence alignment is given in Table 3G, with GPCR3 being shown on line 4, in a ClustalW analysis comparing GPCR3 with related protein sequences.

TABLE 3G
Information for the ClustalW proteins:

1. Hylobates liar (Common Gibbon) OLF, SPTREMBL -Acc # O77758, SEQ ID NO:43
2. Pan troglodytes (Chimpanzee) OLF, SPTREMSL-Acc # O77756, SEQ ID NO:41
3. Mus musculus OLF, GENBANK-Acc # AAF20365, SEQ ID NO:42
4. Novel Human_OLF, GPCR3, SEQ ID NO:8

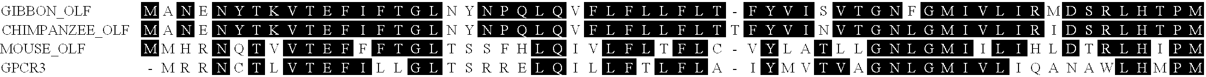

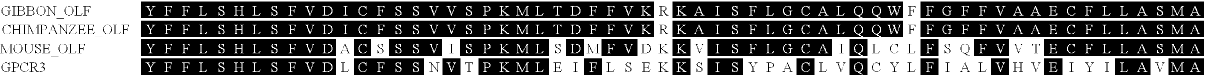

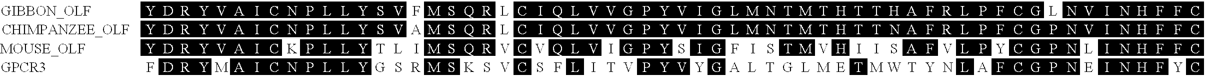

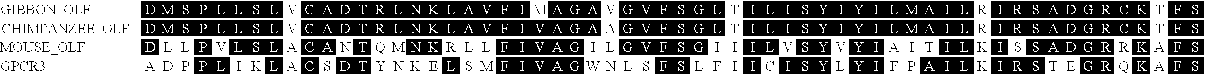

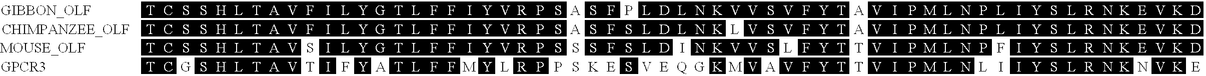

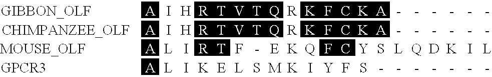

DOMAIN results for GPCR3 were collected from the Conserved Domain Database (CDD) with Reverse Position Specific BLAST. This BLAST samples domains found in the Smart and Pfam collections. The results are listed in Table 3H with the statistics and domain description.

TABLE 3H
DOMAIN results for GPCR3.

```
gnl|Pfam|pfam00O01, 7tm_1, 7 transmembrane receptor
(rhodopsin family) (SEQ ID NO:29)
                                      Length = 377
                                      Score = 92.8 bits (229), Expect = 2e-20
```

| | | |
|---|---|---|
| Query: 40 | GNLGMIVLIQANAWLHNPMYFFLSHLSFVDLCFSSNVTPKMLEIFLSEKKSISYPACLVQ | 99 |
| Sbjct: 1 | GNVLVCMAVSREKALQTTTNYLIVSLAVADLLVATLVMPWVVYLEVVGEWKFSRIECDIF | 60 |
| | **: : : : * : : *: ** :: * * :: : : : * * : | |
| Query: 100 | CYLFIALVHVEIYILAVMAFDRYMAICNPLLYGSRN-SKSVCSFLITVPYVYGALTGLNE | 158 |
| Sbjct: 61 | VTLDVMNCTASILNLCAISIDRYTAVANPMLYNTRYSSKRRVTVMIAIVWVLSFTISCPM | 120 |
| | * : : * * :: *** *: *:** :* ** : :* : :* | |
| Query: 159 | TMWTYNLAFCGPNEINHFYCADPPLIKLACSDTYNKELSNFIVAGWNLSFSLFIICISYL | 218 |
| Sbjct: 121 | LFGLNNTDQNE------------------CIIANPAFVVYSSIVS--FYVPFIVTLLVYI | 160 |
| | * * : : : *: | |
| Query: 219 | YIFPAILKIRSTEGRQK | 235 |
| Sbjct: 161 | KIYIVLRRRRXRVNTKR | 177 |
| | *: : : * | :: |

The nucleic acids and proteins of the invention are useful in potential therapeutic applications implicated in various in various GPCR-related pathological disorders and/or OR-related pathological disorders, described further below. For example, a cDNA encoding the olfactory receptor-like protein may be useful in gene therapy, and the olfactory receptor-like protein may be useful when administered to a subject in need thereof. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Other GPCR-related diseases and disorders are contemplated.

The novel nucleic acid encoding Olfactory receptor-like protein, and the Olfactory receptor-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. This novel protein also has immense value in development of powerful assay system for functional analysis of various human disorders, which will help in understanding of pathology of the disease and development of new drug targets for various disorders.

GPCR4

GPCR4 is an Olfactory Receptor (“OR”)-like protein, wherein two alternative novel GPCR4 nucleic acids and encoded polypeptides are disclosed.

The novel GPCR4a nucleic acid of 980 nucleotides (also referred to as APOO 1112₁₃ B) is shown in Table 4A. An ORF begins with an ATG initiation codon at nucleotides 19-21 and ends with a TAA codon at nucleotides 940-42. A putative untranslated region upstream from the initiation codon and downstream from the termination codon is underlined in Table 4A, and the start and stop codons are in bold letters.

TABLE 4A
GPCR4a Nucleotide Sequence (SEQ ID NO:9)

TGACTTAGAATTCAGAAAATGCTCAATTTCACCGATGTGACAGAGTTCATTCTTTTGGGGCTAACGAGCCGTCGAGAATGGCA
AGTTCTCTTCTTCATCATCTTTCTTGTGGTCTACATCATCACCATGGTGGGCAATATCGGCATGATGGTGTTAATCAAGGTCA
GTCCTCAGCTTAACAACCCCATGTACTTTTTCCTCAGTCACTTGTCATTTGTTGATGTGTGGTTTTCTTCCAATGTCACCCCT
AAAATGTTGGAAAACCTGTTTTCAGATAAAAAAACAATTACTTATGCTGGTTGTTTAGTACAGTGTTTCTTCTTCATTGCTCT
TGTCCATGTGGAAATTTTTATTCTTGCTGCGATGGCCTTTGATAGATACATGGCAATTGGGAATCCTCTGCTTTATGGCAGTA
AAATGTCAAGGGTTGTCTGTATTCGACTGATTACTTTCCCTTACATTTATGGTTTTCTGACGAGTCTGGCAGCAACATTATGG
ACTTACGGCTTGTACTTCTGTGGAAAAATTGAGATCAACCATTTCTACTGTGCAGATCCACCTCTCATCAAAATGGCCTGTGC
CGGGACCTTTGTAAAAGAATATACAATGATCATACTTGCCGGCATTAACTTCACATATTCCCTGACTGTAATTATCATCTCTT
ACTTATTCATCCTCATTGCCATTCTGCGAATGCGCTCAGCAGAAGGAAGGCAGAAGGCCTTTTCCACATGTGGGTGGGATCTG
ACAGCTGTCATTATATTCTATGGTACTCTGATCTTCATGTATCTCAGACGTCCCACAGAGGAGTCTGTGGAGCAGGGGAAGAT
GGTGGCTGTGTTCTATACCACAGTGATCCCCATGTTGAATCCCATGATCTACAGTCTGAGGAACAAGGATGTGAAAAAGGCCA
TGATGAAAGTGATCAGCAGATCATGTTAAACAAAATAAAATCAAATTTGATTTAATTTTATCTTCTA

The GPCR4a protein encoded by SEQ ID NO:9 has 307 amino acid residues and is presented using the one-letter code in Table 4B. The Psort profile for GPCR4 predicts that this sequence has a signal peptide and is likely to be localized at the plasma membrane with a certainty of 0.6000. The most likely cleavage site for a peptide is between amino acids 39 and 40, i.e., at the dash in the amino acid sequence MVG-NIG, based on the SignalP result.

TABLE 4B
Encoded GPCR4a protein sequence (SEQ ID NO:1O).

MLNFTDVTEFILLGLTSRREWQVLFFIIFLVVYIITMVGNIGMMVLIKVSPQLNNPMYFFLSHLSFVDVWFSSNVTPKMLENL
FSDKKTITYAGCLVQCFFFIALVHVEIFILAAMAFDRYMAIGNPLLYGSKMSRVVCIRLITFPYIYGFLTSLAATLWTYGLYF
CGKIEINHFYCADPPLIKMACAGTFVKEYTMIILAGINFTYSLTVIIISYLFILIAILRMRSAEGRQKAFSTCGSHLTAVIIF
YGTLIFMYLRRPTEESVEQGKMVAVFYTTVIPMLNPMIYSLRNKCDVKKAMMKVISRSC

The target GPCR4a sequence was subjected an the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a library containing a wide range of cDNA species. The resulting amplicon was gel purified, cloned and sequenced to high redundancy. The 980 nucleotides of GPCR4b (also referred to as AC020597A) are provided in Table 4C. The resulting GPCR4b nucleotide sequence differs from that of GPCR4a at nine positions, namely A75G, A100G, C102T, C264T, T270A, C582T, A610C, T627C and T759C.

TABLE 4C
GPCR4b Nucleotide Sequence (SEQ ID NO:11)

TGACTTAGAATTCAGAAAATGCTCAATTTCACCGATGTGACAGAGTTCATTCTTTTGGGGCTAACGAGCCGTCGGGAATGGCA
AGTTCTCTTCTTCATCGTTTTTCTTGTGGTCTACATCATCACCATGGTGGGCAATATCGGCATGATGTTGTTAATCAAGGTCA
GTCCTCAGCTTAACAACCCCATGTACTTTTTCCTCAGTCACTTGTCATTTGTTGATGTGTGGTTTTCTTCCAATGTCACCCCT
AAAATGTTGGAAAATCTGTTATCAGATAAAAAAACAATTACTTATGCTGGTTGTTTAGTACAGTGTTTCTTCTTCATTGCTCT
TGTCCATGTGGAAATTTTTATTCTTCCTGCGATGGCCTTTGATAGATACATGGCAATTGGGAATCCTCTGCTTTATGGCAGTA
AAATGTCAAGGGTTGTCTGTATTCGACTGATTACTTTCCCTTACATTTATGGTTTTCTGACGAGTCTGGCAGCAACATTATGG
ACTTACGGCTTGTACTTCTGTGGAAAAATTGAGATCAACCATTTCTACTGTGCAGATCCACCTCTCATCAAAATGGCCTGTGC
TGGGACCTTTGTAAAAGAATATACAATGCTCATACTTGCCGGCATCAACTTCACATATTCCCTGACTGTAATTATCATCTCTT
ACTTATTCATCCTCATTGCCATTCTGCGAATGCGCTCAGCAGAAGGAAGGCAGAAGGCCTTTTCCACATGTGGGTCCCATCTG
ACAGCTGTCATCATATTCTATGGTACTCTGATCTTCATGTATCTCAGACGTCCCACAGAGGAGTCTGTGGAGCAGGGGAAGAT
GGTGGCTGTGTTCTATACCACAGTGATCCCCATGTTGAATCCCATGATCTACAGTCTGAGGAACAAGGATGTGAAAAAGGCCA
TGATGAAAGTGATCAGCAGATCATGTTAAACAAAATAAAATCAAATTTGATTTAATTTTATCTTCTA

The GPCR4b protein encoded by SEQ ID NO: 11 has 314 amino acid residues and a molecular weight of 35155.8 Daltons, as presented using the one-letter code in Table 4D. GPCR4a differs from GPCR4b at four residues, namely 128V, V45L, F84L and 1198L. The signal peptide and Psort analyses for both GPCR4 variants are the same.

TABLE 4D
Encoded GPCR4b protein sequence (SEQ ID NO:12)
MLNFTDVTEFILLGLTSRREWQVLFFIVFLVVYIITMVGNIGMMLLIKVSPQLNNPMYFFLSHLSFVDVWFSSNVTPKMLENL
LSDKKTITYAGCLVQCFFFIALVHVEIFILAAMAFDRYMATGNPLLYGSKMSRVVCIRLITFPYIYGFLTSLAATLWTYGLYF
CGKIEINHFYCADPPLIKMACAGTFVKEYTMLILAGINFTYSLTVIIISYLFILIAILRMRSAEGRQKAFSTCGSHLTAVIIF
YGTLIFMYLRRPTEESVEQGKWAVFYTTVIPMLNPMIYSLRNKDVKKAMMKVISRSC

Unless specifically addressing GPCR4a or 4b, assume any reference to GPCR4 to encompass all variants.

In a search of sequence databases, it was found, for example, that the nucleic acid sequence for GPCR4a has 591 of 940 bases (62%) identical to and 591 of 940 bases (62%) positive with Rattus norvegicus species taste bud Receptor clone (GENBANK-ID: U50948) (SEQ ID NO:44) (Table 4E). The residues that differs between GPCR4a and GPCR4b are highlighted in black and marked with the (o) symbol.

TABLE 4E
BLASTN of GPCR4a against rat TB567 gene

>gb:GENBANK-ID:RNU50948|acc:U50948 Rattus norvegicus taste bud receptor protein TB
567 (TB 567) gene, complete cds - Rattus norvegicus, 1299 bp. (SEQ ID NO:44)
Score = 1221 (183.2 bits), Expect = 3.3e-49, P = 3.3e-49
Identities = 591/940 (62%), Positives = 591/940 (62%), Strand = Plus / Plus

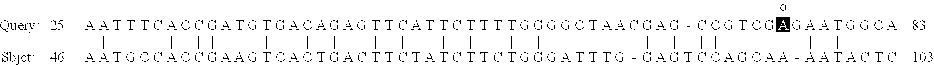

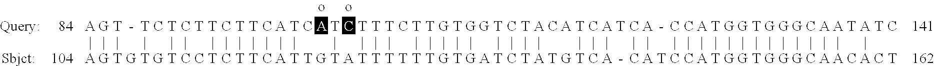

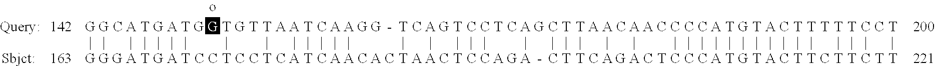

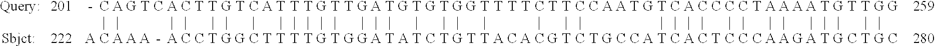

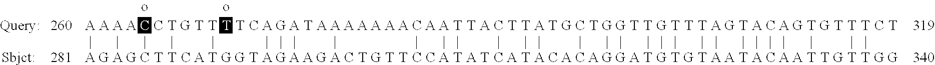

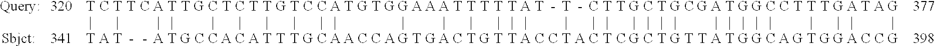

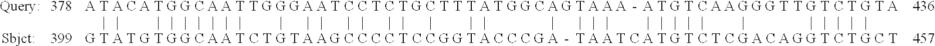

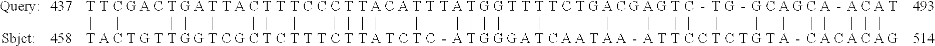

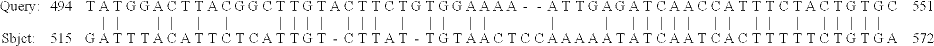

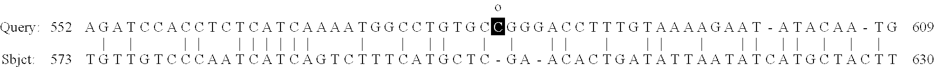

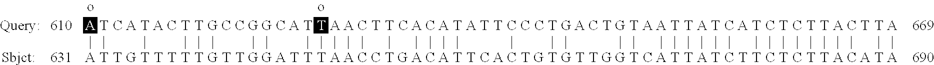

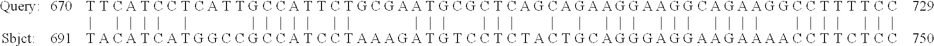

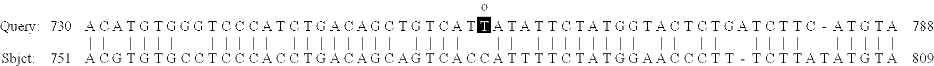

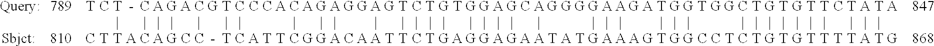

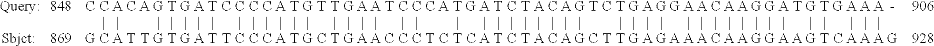

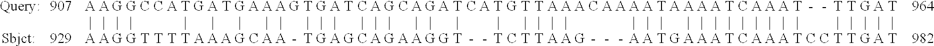

BLASTN alignments also found homology between two fragments of GPCR4 and Mus musculus odorant receptor M72 (GENBANK-ID:AF247656) shown if Table 4F. M72 residues 821-890 (SEQ ID NO:45) and residues 160-201 (SEQ ID NO:46), are aligned with GPCR4 in Table 4F.

TABLE 4F
BLASTN of GPCR4a against M72

>gb|AF247656.1|AF247656 Mus musculus odorant receptor M72 (M72) gene, complete cds
Length = 930
Score = 83.8 bits (42), Expect = 2e-13
Identities = 63/70 (90%), Strand = Plus/Plus

| | | |
|---|---|---|
| Query: | 836 ctgtgttctataccacagtgatccccatgttgaatcccatgatctacagtctgaggaaca | 895 |
| | \|\|\|\|\|\|\|\|\|\| \|\|\|\|\|\|\|\|\|\|\|\|\|\|\|\|\|\|\|\| \|\| \|\|\| \|\|\|\|\|\|\|\|\|\| \|\|\|\|\| \|\|\|\| | |
| Sbjct: | 821 ctgtgttctacaccacagtgatccccatgttcaaccccctgatctacagcctgagaaaca | 880 |
| Query: | 896 aggatgtgaa | 905 |
| | \|\|\|\| \|\|\|\|\| | |
| Sbjct: | 881 aggaggtgaa | 890 (SEQ ID NO:45) |

Score = 44.1 bits (22), Expect = 0.13
Identities = 37 42 (88%), Strand = Plus Plus

| | | |
|---|---|---|
| Query: | 172 cagcttaacaaccccatgtactttttcctcagtcacttgtca | 213 |
| | \|\|\|\|\|\| \|\|\| \|\|\|\|\|\|\|\|\|\|\|\| \|\|\|\|\|\|\|\|\| \|\| \|\|\|\|\| | |
| Sbjct: | 160 cagcttcacacccccatgtacttcttcctcagtaacctgtca | 201 (SEQ ID NO:46) |

BLASTN alignments found homology between fragments of GPCR4 and Mus musculus odorant receptor K42 (GENBANK-ID: AE282291)(SEQ ID NO:47) shown if Table 4G.

TABLE 4G
BLASTN of GPCR4b against OR K42

>gb|AF282291.1|AF282291 Mus musculus odorant receptor K42 gene, complete cds
(SEQ ID NO:47) Length = 927
Score = 77.8 bits (39), Expect = 1e-11
Identities = 60/67 (89%); Strand = Plus/Plus

| | | |
|---|---|---|
| Query: | 836 ctgtgttctataccacagtgatccccatgttgaatcccatgatctacagtctgaggaaca | 895 |
| | \|\|\|\|\|\|\|\|\|\| \|\|\|\|\|\|\|\|\|\|\|\|\|\| \|\|\| \| \|\|\|\|\|\| \| \|\| \|\|\|\|\|\|\|\|\|\|\|\|\|\|\|\| | |
| Sbjct: | 815 ctgtgttctacaccacagtgatcccaatgctaaatcccctcatatacagtctgaggaaca | 874 |
| Query: | 896 aggatgt | 902 |
| | \|\|\|\|\|\|\| | |
| Sbjct: | 875 aggatgt | 881 |

BLASTN alignments found homology between GPCR4 and Mus musculus odorant receptor K40 (GENBANK-ID:AF282289) (SEQ ID NO:48) shown in Table 4G.

TABLE 4H
BLASTN of GPCR4 against OR K40

>gb|AF282289.1|AF282289 Mus musculus odorant receptor K40 gene, complete cds
(SEQ ID NO:48) Length = 927
Score = 75.8 bits (38), Expect = 4e-11
Identities = 62/70 (88%); Strand = Plus/Plus

| | | |
|---|---|---|
| Query: | 836 ctgtgttctataccacagtgatccccatgttgaatcccatgatctacagtctgaggaaca | 895 |
| | \|\|\|\|\|\|\|\|\|\|\|\|\|\|\|\|\|\|\|\|\|\|\|\|\|\|\|\|\| \|\|\|\| \|\| \| \|\| \|\|\|\|\|\| \|\|\|\|\|\|\|\|\| | |
| Sbjct: | 824 ctgtgttctataccacagtgatccccatgctgaacccattaatatacagtttgaggaaca | 883 |
| Query: | 896 aggatgtgaa | 905 |
| | \| \|\|\|\|\|\|\|\| | |
| Sbjct: | 884 aagatgtgaa | 893 |

The full GPCR4a amino acid sequence has 155 of 304 amino acid residues (50%) identical to, and 215 of 304 residues (70%) positive with, the 314 amino acid residue proteins from Pan troglodytes OR93CH (ptnr: SPTREMBL-ACC: 077756) (SEQ ID NO:41) (Table 4I). The residue that differs between GPCR4a and GPCR4b are highlighted in black and marked with the (o) symbol.

TABLE 4I
BLASTX of GPCR4a against OR93CH

>ptnr:SPTREMBL-ACC:O77756 OLFACTORY RECEPTOR OR93CH - Pan troglodytes (Chimpanzee),
314 aa. (SEQ ID NO:41)
Score = 806 (283.7 bits), Expect = 2.0e-79, P = 2.0e-79
Identities = 155/304 (50%), Positives = 215/304 (70%), Frame = +1

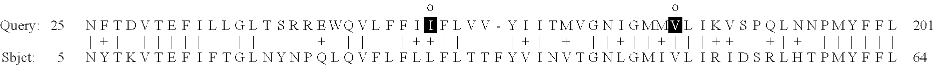

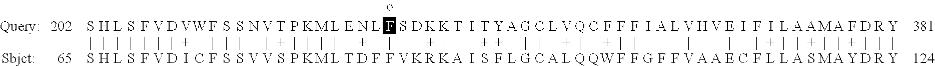

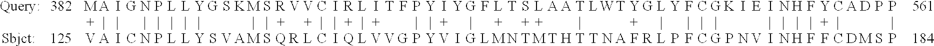

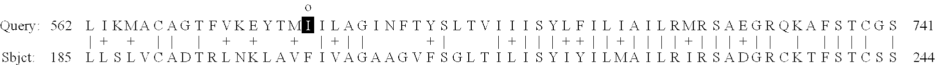

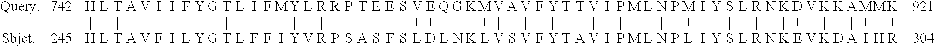

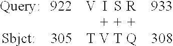

The GPCR4a amino acid sequence has 139 of 303 amino acid residues (45%) identical to, and 192 of 303 residues (62%) positive with the 303 amino acid OR93Gib protein from Hylobates lar (GENBANK-ID:AAC63971.1) (SEQ ID NO:43) (Table 4J). The residue that differs between GPCR4a and GPCR4b are highlighted in black and marked with the (o) symbol.

TABLE 4J
BLASTX of GPCR4a against OR93Gib

>gb|AAC63971.1| (AF045580) olfactory receptor OR93Gib [Hylobates lar]
(SEQ ID NO:43) Length = 313
Score = 272 bits (695), Expect = 4e-72
Identities = 155/303 (51%), Positives = 208/303 (69%)

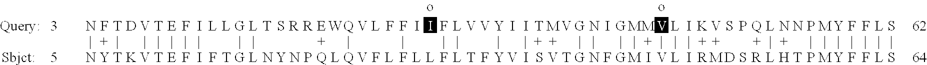

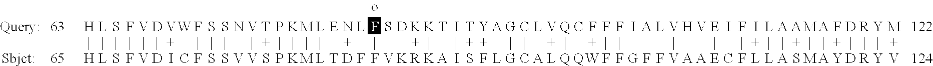

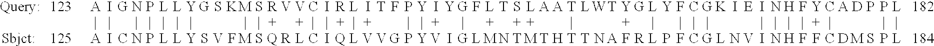

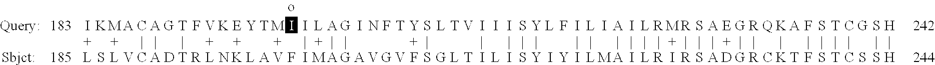

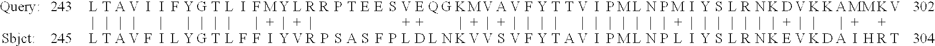

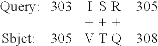

The GPCR4a protein has 137 of 304 amino acid residues (45%) identical to, and 191 of 304 residues (62%) positive with, the 308 amino acid odorant receptor K42 from Mus musculus (GENBANK-ID:AAG39876.1) (SEQ ID NO:49) (Table 4K). The residue that differs between GPCR4a and GPCR4b are highlighted in black and marked with the (o) symbol.

TABLE 4K
BLASTX of GPCR4a against K42

| >gb\|AAG39876.1\| (AF282291) odorant receptor K42 [MUS musculus (SEQ ID NO:49), 308 aa | |
|---|---|
| Statistics for GPCR4a: | Score = 271 bits (694), Expect = 6e-72 |
| | Identities = 149/304 (49%), Positives = 203/304 (67%) |
| Statistics for GPCR4b: | Score = 273 bits (699), Expect = 2e-72 |
| | Identities = 138/304 (45%), Positives = 192/304 (62%) |

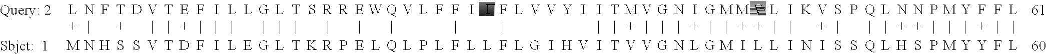

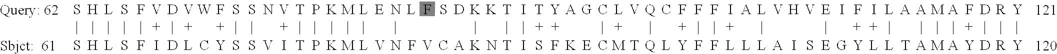

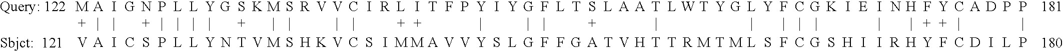

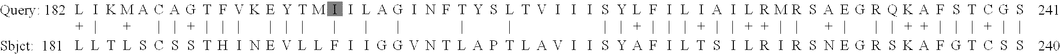

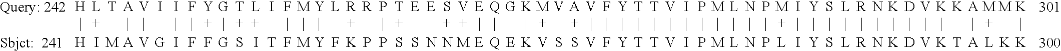

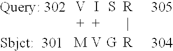

The GPCR4b protein has 141 of 301 amino acid residues (46%) identical to, and 189 of 301 residues (61%) positive with, the 314 amino acid OR 511 from Mus musculus (GENBANK-ID:AAG39876.1) (SEQ ID NO:50) (Table 4L). The residue that differs between GPCR4a and GPCR4b are highlighted in black and marked with the (o) symbol.

TABLE 4L
BLASTX of GPCR4b against OR 5I1

ref|NP_006628.1| olfactory receptor, family 5, subfamily I, member 1 [Homo sapiens]
sp|Q13606|O5I1_HUMAN OLFACTORY RECEPTOR 5I1 (OLFACTORY RECEPTOR-LIKE PROTEIN OLF1)
gb|AAB01214.1| (U56420) HsOLF1 [Homo sapiens] (SEQ ID NO:50); Length = 314
Score = 274 bits (700), Expect = 1e-72
Identities = 141/301 (46%), Positives = 189/301 (61%)

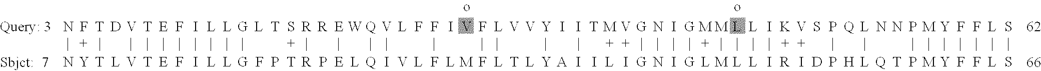

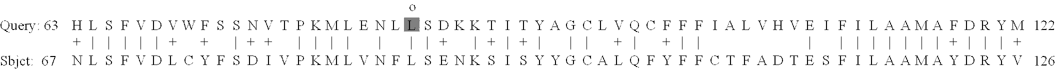

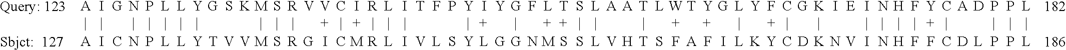

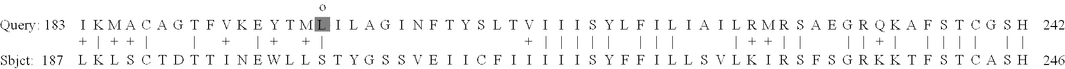

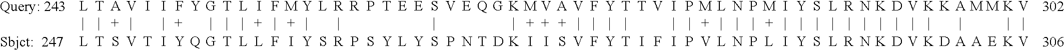

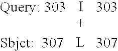

The full amino acid sequence of the GPCR4b protein has 140 of 303 amino acid residues (46%) identical to, and 193 of 303 residues (63%) positive with, the 312 amino acid OR4 protein from Mus musculus (GENBANK-ID:AAG39876.1) (SEQ ID NO:34) (Table 4J). The residue that differs between GPCR4a and GPCR4b are highlighted in black and marked with the (o) symbol.

TABLE 4M
BLASTX of GPCR4b and OR 4

emb|CAA64370.1| (X94744) olfactory receptor 4 [Gallus gallus]; (SEQ ID NO:34)
Length = 312; Score = 271 bits (693), Expect = 7e-72
Identities = 140/303 (46%), Positives = 193/303 (63%)

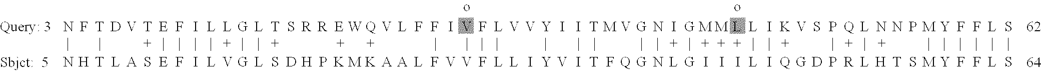

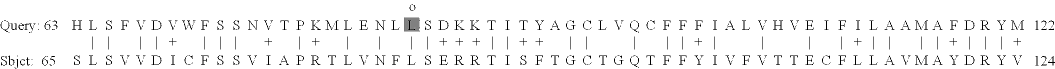

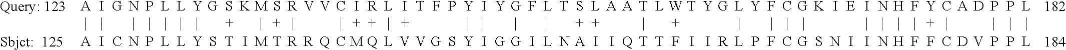

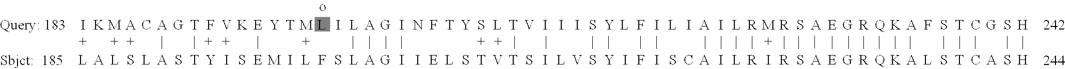

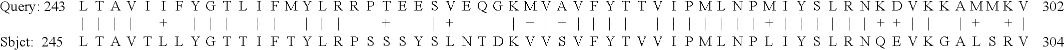

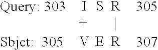

A multiple sequence alignment is given in Table 4K, with GPCR4a being shown on line 1, in a ClustalW analysis comparing GPCR4a with related protein sequences.

TABLE 4N
Information for the ClustalW proteins:

1. Novel Human_OLF, GPCR4a, SEQ ID NO:10
2. Hylobates lar (Common Gibbon) OLF, SPTREMBL-Acc # O77758, SEQ ID NO:43
3. Pan troglodytes (Chimpanzee) OLF, SPTREMBL-ACC # O77756, SEQ ID NO:41
4. Mus musculus OLF, GENBANK-Acc # AAF20365, SEQ ID NO:42
5. Homo sapiens OLF, SWISSPROT-Acc # Q13606, SEQ ID NO:33

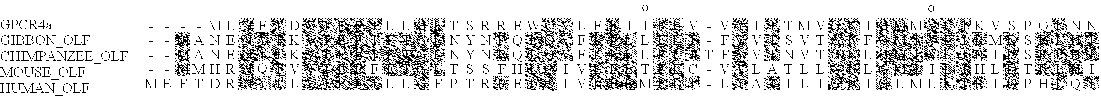

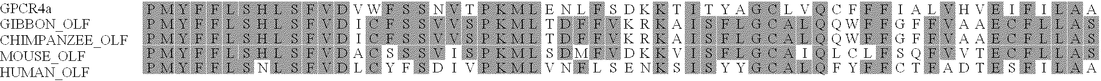

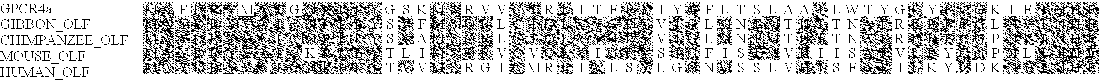

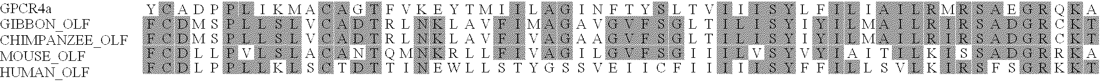

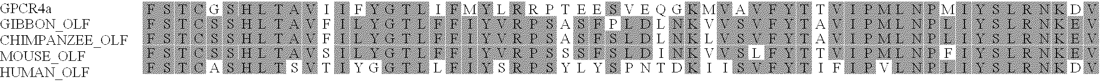

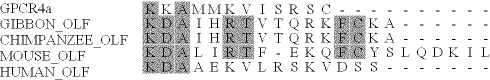

DOMAIN results for GPCR4a were collected from the Conserved Domain Database (CDD) with Reverse Position Specific BLAST. This BLAST samples domains found in the Smart and Pfam collections. The results are listed in Table 4L with the statistics and domain description. Residues 1-163 (SEQ ID NO:29) and residues 313-377 (SEQ ID NO:37) of 7tm_{—}1 are aligned with GPCR4 in Table 40. The residue that differs between GPCR4a and GPCR4b are highlighted in black and marked with the (o) symbol.

TABLE 4O
DOMAIN results for GPCR4a.

gnl|Pfam|pfam00001, 7tm_1, 7 transmembrane receptor (rhodopsin family)
(SEQ ID NO: 29) Length = 377
Statistics for GPCR4a: Score = 89.7 bits (221), Expect = 2e-19
Statistics for GPCR4b: Score = 90.5 bits (223), Expect = 1e-19

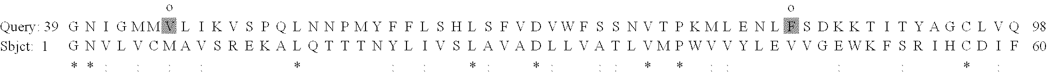

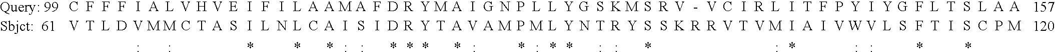

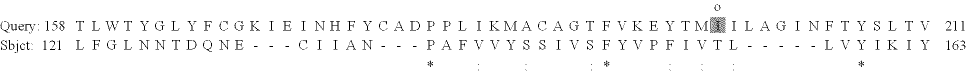

Sbjct: 7 transmembrane receptor (rhodopsin family) fragment (SEQ ID NO: 37)
gnl|Pfam|pfam00001, 7tm_1, 7 transmetnbrane receptor (rhodopsin family) 377 aa
Statistics for GPCR4a: Score = 40.4 bits (93). Expect = 1e-04
Statistics for GPCR4b: Score = 40.4 bits (93), Expect = 1e-04

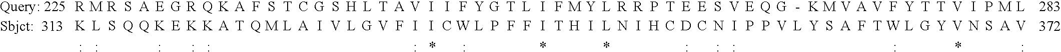

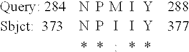

The nucleic acids and proteins of GPCR4 are useful in potential therapeutic applications implicated in various in various GPCR-related pathological disorders and/or OR-related pathological disorders, described further below. For example, a cDNA encoding the olfactory receptor-like protein may be useful in gene therapy, and the olfactory receptor-like protein may be useful when administered to a subject in need thereof. By way of nonlimiting example, the compositions of the present invention will have efficacy for treatment of patients suffering from neoplasm, adenocarcinoma, lymphoma, prostate cancer, uterus cancer, immune response, AIDS, asthma, Crohn's disease, multiple sclerosis, and Albright Hereditary Ostoeodystrophy. Other GPCR-related diseases and disorders are contemplated.

The novel GPCR4 nucleic acid and protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. This novel protein also has immense value in development of powerful assay system for functional analysis of various human disorders, which will help in understanding of pathology of the disease and development of new drug targets for various disorders.

GPCR5

GPCR5 is an Olfactory Receptor (“OR”)-like protein, wherein three alternative novel GPCR5 nucleic acids and encoded polypeptides are disclosed.

The novel GPCR5a nucleic acid of 980 nucleotides (also referred to as AP001112_C) is shown in Table 5A. An ORF begins with an ATG initiation codon at nucleotides 26-28 and ends with a TGA codon at nucleotides 941-43. A putative untranslated region upstream from the initiation codon and downstream from the termination codon is underlined in Table 5A, and the start and stop codons are in bold letters.

TABLE 5A
GPCR5a Nucleotide Sequence (SEQ ID NO:13)

AGCTTGAAGAGCAAACTGTCAGGAAATGTCCAACACAAATGGCAGTGCAA
TCACAGAATTCATTTTACTTGGGCTCACAGATTGCCCGGAACTCCAGTCT
CTGCTTTTTGTGCTGTTTCTGGTTGTTTACCTCGTCACCCTGCTAGGCAA
CCTGGGCATGATAATGTTAATGAGACTGGACTCTCGCCTTCACACGCCCA
TGTACTTCTTCCTCACTAACTTAGCCTTTGTGGATTTGTGCTATACATCA
AATGCAACCCCGCAGATGTCGACTAATATCGTATCTGAGAAGACCATTTC
CTTTGCTGGTTGCTTTACACAGTGCTACATTTTCATTGCCCTTCTACTCA
CTGAGTTTTACATGCTGGCAGCAATGGCCTATGACCGCTATGTGGCCATA
TATGACCCTCTGCGCTACAGTGTGAAAACGTCCAGGAGAGTTTGCATCTG
CTTGGCCACATTTCCCTATGTCTATGGCTTCTCAGATGGACTCTTCCAGG
CCATCCTGACCTTCCGCCTGACCTTCTGTAGATCCAATGTCATCAACCAC
TTCTACTGTGCTGACCCGCCGCTCATTAAGCTTTCTTGTTCTGATACTTA
TGTCAAAGAGCATGCCATGTTCATATCTGCTGGCTTCAACCTCTCCAGCT
CCCTCACCATCGTCTTGGTGTCCTATGCCTTCATTCTTGCTGCCATCCTC
CGGATCAAATCAGCAGAGGGAAGGCACAAGGCATTCTCCACCTGTGGTTC
CCATATGATGGCTGTCACCCTGTTTTATGGGACTCTCTTTTGCATGTATA
TAAGACCACCAACAGATAAGACTGTTGAGGAATCTAAAATAATAGCTGTC
TTTTACACCTTTGTGAGTCCGGTACTTAATCCATTGATCTACAGTCTGAG
GAATAAAGATGTGAAGCAGGCCTTGAAGAATGTCCTGAGATGAAATATTG
TCATGACCATGGTGATGCCTTTGTTTCCTA

The GPCR5a protein encoded by SEQ ID NO:13 has 305 amino acid residues, and is presented using the one-letter code in Table 5B. The SignalP, Psort and/or Hydropathy profile for GPCR5a predict that GPCR5a has a signal peptide and is likely to be localized at the plasma membrane with a certainty of 0.6000. The signalP shows a signal sequence is coded for in the first 44 amino acids, i.e., with a cleavage site at the dash in the sequence NLG-MIM, between amino acids 44 and 45. This is typical of this type of membrane protein.

TABLE 5B
Encoded GPCRSa protein sequence (SEQ ID NO:14).
MSNTNGSAITEFILLGLTDCPELQSLLFVLFLVVYLVTLLGNLGMIMLMRLDSRLHTPMYFFLTNLAFVDLCYTSNATPQMST
NIVSEKTISFAGCFTQCYIFIALLLTEFYMLAAMAYDRYVAIYDPLRYSVKTSRRVCICLATFPYVYGFSDGLFQAILTFRLT
FCRSNVINHFYCADPPLIKLSCSDTYVKEHAMFISAGFNLSSSLTIVLVSYAFILAAILRIKSAEGRHKAFSTCGSHMMAVTL
FYGTLFCMYIRPPTDKTVEESKIIAVFYTFVSPVLNPLIYSLRNKDVKQALKNVLR

The target GPCR5a sequence, above, was subjected an the exon linking process to confirm the sequence, as reported for GPCR2 and GPCR4, above. The resulting GPCR5b sequence (also referred to herein as AC0170103B1) is reported below in Table 5C .

TABLE 5C
GPCR5 Nucleotide Sequence (SEQ ID NO:15)

AGCTTGAAGAGCAAACTGTCAGGAAATGTCCAACACAAATGGCAGTGCAATCACAGAATTCATTTTACTTGGGCTCACAGATT
GCCCGGAACTCCAGTCTCTGCTTTTTGTGCTGTTTCTGGTTGTTTACCTCGTCACCCTGCTAGGCAACCTGGGCATGATAATG
TTAATGAGACTGGACTCTCGCCTTCACACGCCCATGTACTTCTTCCTCACTAACTTAGCCTTTGTGGATTTGTGCTATACATC
AAATGCAACCCCGCAGATGTCGACTAATATCGTATCTGAGAAGACCATTTCCTTTGCTGGTTGCTTTACACAGTGCTACATTT
TCATTGCCCTTCTACTCACTGAGTTTTACATGCTGGCAGCAATGGCCTATGACCGCTATGTGGCCATATATGACCCTCTGCGC
TACAGTGTGAAAACGTCCAGGAGAGTTTGCATCTGCTTGGCCACATTTCCCTATGTCTATGGCTTCTCAGATGGACTCTTCCA
GGCCATCCTGACCTTCCGCCTGACCTTCTGTAGATCCAGTGTCATCAACCACTTCTACTGTGCTGACCCGCCGCTCATTAAGC
TTTCTTGTTCTGATACTTATGTCAAAGAGCATGCCATGTTCATATCTGCTGGCTTCAACCTCTCCAGCTCCCTCACCATCGTC
TTGGTGTCCTATGCCTTCATTCTTGCTGCCATCCTCCGGATCAAATCAGCAGAGGGAAGGCACAAGGCATTCTCCACCTGTGG
TTCCCATATGATGGCTGTCACCCTGTTTTATGGGACTCTCTTTTGCATGTATATAAGACCACCAACAGATAAGACTGTTGAGG
AATCTAAAATAATAGCTGTCTTTTACACCTTTGTGAGTCCGGTACTTAATCCATTGATCTACAGTCTGAGGAATAAAGATGTG
AAGCAGGCCTTGAAGAATGTCCTGAGATGAAATATTGTCATGACCATGGTGATGCCTTTGTTTCCTA

The GPCR5b protein encoded by SEQ ID NO:15 has 305 amino acid residues and is presented using the one-letter code in Table 5D. The SignalP, Psort and/or Hydropathy profiles for GPCR5b are the same as for GPCR5a.

TABLE 5D
Encoded GPCR5 protein (SEQ ID NO:16).

MSNTNGSAITEFILLGLTDCPELQSLLFVLFLVVYLVTLLGNLGMIMLMRLDSRLHTPMYFFLTNLAFVDLCYTSNATPQMST
NIVSEKTISFAGCFTQCYIFIALLLTEFYMLAAMAYDRYVAIYDPLRYSVKTSRRVCICLATFPYVYGFSDGLFQAILTFRLT
FCRSSVINHFYCADPPLIKLSCSDTYVKEHAMFISAGFNLSSSLTIVLVSYAFILAAILRIKSAEGRHKAFSTCGSHMMAVTL
FYGTLFCMYIRPPTDKTVEESKIIAVFYTFVSPVLNPLIYSLRNKDVKQALKNVLR

In an alternative embodiment, a novel GPCR5c nucleic acid of 1006 nucleotides (also referred to herein as CG50173-01) is shown in Table 5E. An ORF was identified beginning with an ATG initiation codon at nucleotides 83-85 and ending with a TGA codon at nucleotides 998-1000. Putative untranslated regions, if any, are found upstream from the initiation codon and downstream from the termination codon.

TABLE 5E
GPCR5c Nucleotide Sequence (SEQ ID NO:17)

AATTCAAATGAACATTAACATGGTATGTGCATTTGTTTATATTGGCTTTATTTCCATAGCTTGAAGAGCAAACTGTCAGGAAA
TGCCCAACACAAATGGCAGTGCAATCACAGAATTCATTTTACTTGGGCTCACAGATTGCCCGGAACTCCAGTCTCTGCTTTTT
GTGCTGTTTCTGGTTGTTTACCTCGTCACCCTGCTAGGCAACCTGGGCATGATAATGTTAATGAGACTGGACTCTCGCCTTCA
CACGCCCATGTACTTCTTCCTCACTAACTTAGCCTTTGTGGATTTGTGCTATACATCAAATGCAACCCCGCAGATGTCGACTA
ATATCGTATCTGAGAAGACCATTTCCTTTGCTGGTTGCTTTACACAGTGCTACATTTTCATTGCCCTTCTACTCACTGAGTTT
TACATGCTGGCAGCAATGGCCTATGACCGCTATGTGGCCATATATGACCCTCTGCGCTACAGTGTGAAAACGTCCAGGAGAGT
TTGCATCTGCTTGGCCACATTTCCCTATGTCTATGGCTTCTCAGATGGACTCTTCCAGGCCATCCTGACCTTCCGCCTGACCT
TCTGTAGATCCAATGTCATCAACCACTTCTACTGTGCTGACCCGCCGCTCATTAAGCTTTCTTGTTCTGATACTTATGTCAAA
GAGCATGCCATGTTCATATCTGCTGGCTTCAACCTCTCCAGCTCCCTCACCATCGTCTTGGTGTCCTATGCCTTCATTCTTGC
TGCCATCCTCCGGATCAAATCAGTAGAGGGAAGGCACAAGGCATTCTCCACCTGTGGTTCCCATATGATGGCTGTCACCCTGT
TTTATGGGACTCTCTTTTGCATGTATATAAGACCACCAACAGATAAGACTGTTGAGGAATCTAAAATAATAGCTGTCTTTTAC
ACCTTTGTGAGTCCGGTACTTAATCCATTGATCTACAGTCTGAGGAATAAAGATGTGAAGCAGGGCTTGAAGAATGTCCTGAG
ATGAAATATT

The GPCR5 c protein encoded by SEQ ID NO: 17 has 305 amino acid residues and is presented using the one-letter code in Table 5F. The SignalP, Psort and/or Hydropathy profiles for GPCR5c are the same as for GPCR5a and GPCR5b.

TABLE 5F
Encoded GPCR5c protein sequence (SEQ ID NO:18).

MPNTNGSAITEFILLGLTDCPELQSLLFVLFLVVYLVTLLGNLGMIMLMRLDSRLHTPMYFFLTNLAFVDLCYTSNATPQMST
NIVSEKTISFAGCFTQCYIFIALLLTEFYMLAAMAYDRYVAIYDPLRYSVKTSRRVCICLATFPYVYGFSDGLFQAILTFRLT
FCRSSVINHFYCADPPLIKLSCSDTYVKEHAMFISAGFNLSSSLTIVLVSYAFILAAILRIKSAEGRHKAFSTCGSHMMAVTL
FYGTLFCMYIRPPTDKTVEESKIIAVFYTFVSPVLNPLIYSLRNKDVKQALKNVLR

GPCR5 variants differ at four nucleotide residues, namely GPCR5a and GPCR5b differ from GPCR5c at T29C, C714T and C921G, while GPCR5a and GPCR5c differ from GPCR5b at A537G. GPCR5 variants differ at four amino acid residues, namely GPCR5a and GPCR5b differ from GPCR5c at S2P, A230V and A299G, while GPCR5a and GPCR5c differ from GPCR5b at NI71S. All numbering is in reference to GPCR5a. Unless specifically addressing GPCR5a or GPCR5b or GPCR5c, assume any reference to GPCR5 to encompass all variants.

In a search of sequence databases, it was found, for example, that the nucleic acid sequence GPCR5a has 633 of 959 bases (66%) identical to and 633 of 959 bases (66%) positive with a Gallus gallus species Olfactory Receptor clone (GENBANK-ID: X94742) (SEQ ID NO:51) (Table 5G). The residue that differs between GPCR5a, GPCR5b and GPCR5c are highlighted in black and marked with the (o) symbol.

TABLE 5G
BLASTN of GPCR5a against OR 2 (SEQ ID NO:51)

| >gb:GENBANK-ID:GGCOR2GEN\|acc:X94742 G. gallus cor2 DNA for olfactory receptor 2 Gallus gallus, 996 bp. (SEQ ID NO:51) | |
|---|---|
| Statistics for GPCR5a: | Score = 1409 (211.4 bits), Expect = 1.4e-57, P = 1.4e-57 |
| | Identities = 633/959 (66%), Positives = 633/959 (66%), |
| Statistics for GPCR5c: | Score = 1379 (206.9 bits), Expect = 2.5e-56, P = 2.5e-56 |
| | Identities = 623/944 (65%), Positives = 623/944 (65%) |
| Strand = Plus / Plus | |

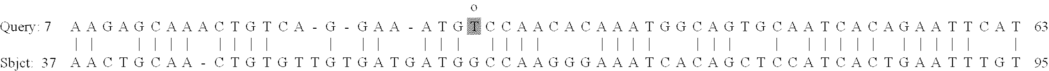

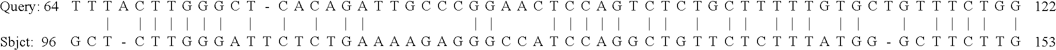

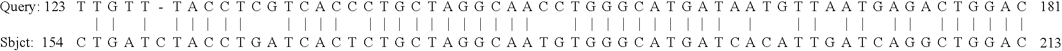

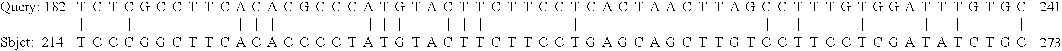

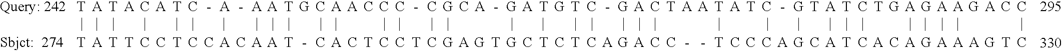

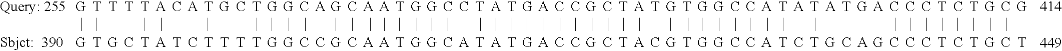

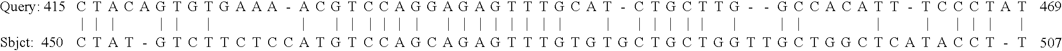

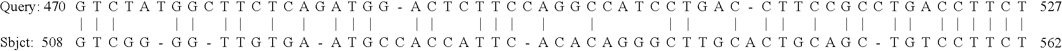

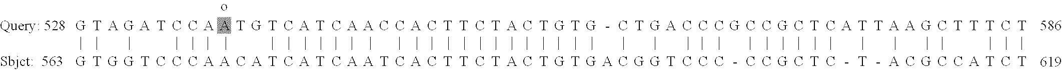

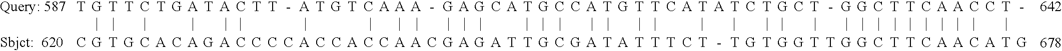

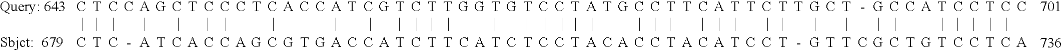

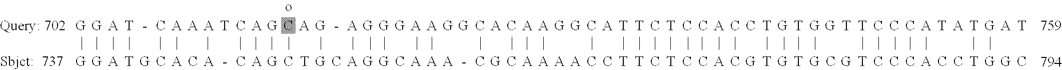

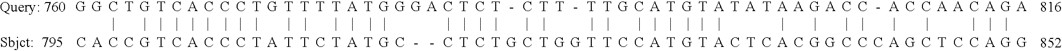

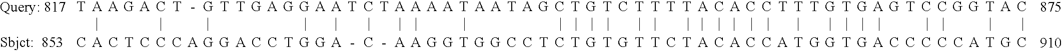

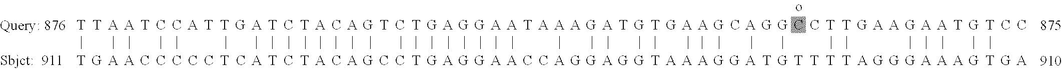

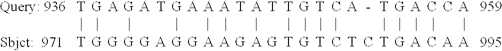

The full amino acid sequence of the protein of GPCR5a has 160 of 301 amino acid residues (53%) identical to, and 215 of 301 residues (71%) positive with, the 313 amino acid OR93GIB from Hylobates lar (ptnr: SPTREMBL-ACC: 077758) (SEQ ID NO:43) (Table 5H). The residue that differs between GPCR5a, GPCR5b and GPCR5c are highlighted in black and marked with the (o) symbol.

TABLE 5H
BLASTX of GPCR5a against OR93GIB

>ptnr:SPTREMBL-ACC:O77758 OLFACTORY RECEPTOR OR93GIB - Hylobates lar (Common gibbon), 313 aa. (SEQ ID NO:43)
Score = 803 (282.7 bits), Expect = 4.2e-79, P = 4.2e-79
Identities = 160/301 (53%), Positives = 215/301 (71%), Frame = +2

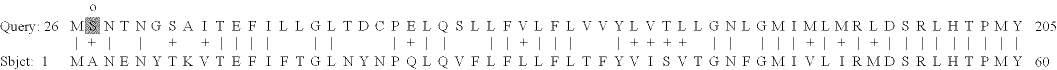

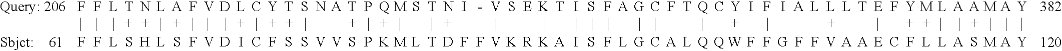

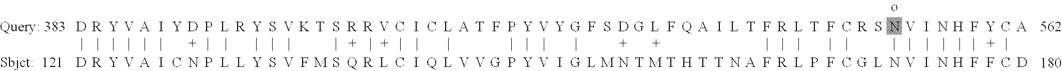

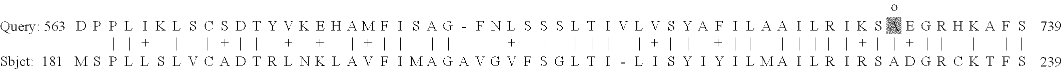

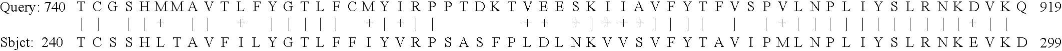

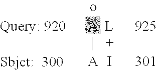

The GPCR5a amino acid has 154 of 306 amino acid residues (55%) identical to, and 199 of 306 residues (64%) positive with, the 309 amino acid M72 from Mus musculus (GENBANK-ID:AAG09870.1) (SEQ ID NO:53) (Table 51). The residue that differs between GPCR5a, GPCR5b and GPCR5c are highlighted in black and marked with the (o) symbol.

TABLE 5I
BLASTX of GPCR5a against M72

>gb|AAG09780.1|AF247656_1 (AF247656) odorant receptor M72 [Mus musculus]
(SEQ ID NO:53) Length = 309
Score = 294 bits (752), Expect = 1e-78
Identities = 161/306 (53%), Positivess = 206/306 (68%), Gaps = 2/306 (0%)

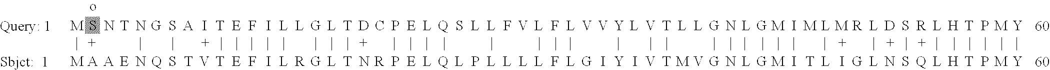

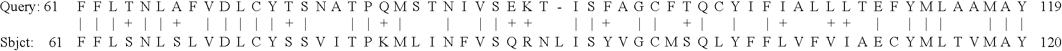

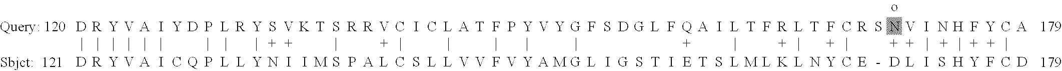

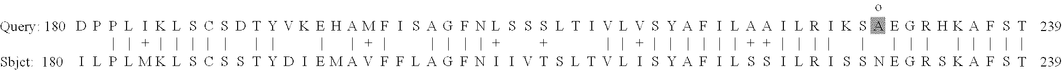

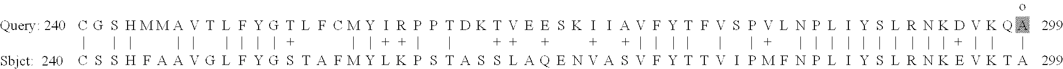

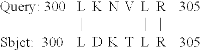

The GPCR5a amino acid has 148 of 301 amino acid residues (49%) identical to, and 198 of 301 residues (65%) positive with, the 308 amino acid K42 from Mus musculus (GENBANK-ID:AAG39876.1) (SEQ ID NO:53) (Table 5J). The residue that differs between GPCR5a, GPCR5b and GPCR5c are highlighted in black and marked with the (o) symbol.

TABLE 5J
BLASTX of GPCR5a against K42

>gb|AAG39876.1|AF282291_1 (AF282291) odorant receptor K42 [Mus musculus] (SEQ ID NO:53) Length = 308
Score = 293 bits (751), Expect = 1e-78
Identities = 153/301 (51%) , Positives = 203/301 (67%) , Gaps = 1/301 (0%)

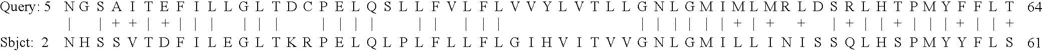

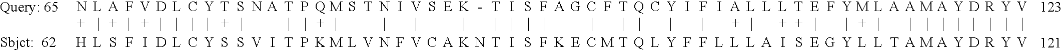

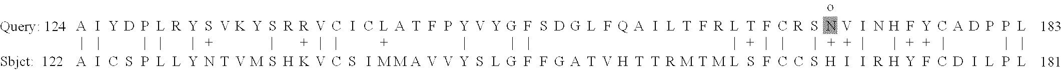

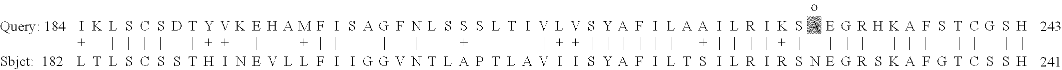

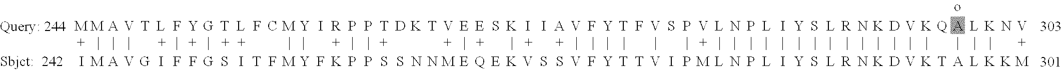

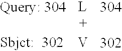

The GPCR5b amino acid sequence has 153 of 306 amino acid residues (50%) identical to, and 198 of 306 residues (64%) positive with, the 309 amino acid M71 from Mus musculus (GENBANK-ID:AAG29379.1) (SEQ ID NO:54) (Table 5K). The residue that differs between GPCR5a, GPCR5b and GPCR5c are highlighted in black and marked with the (o) symbol.

TABLE 5K
BLASTX of GPCR5b against M71

>gb|AAG29379.1|AF281061_1 (AF281061) odorant receptor M71 [Mus musculus] (SEQ ID NO:54) Length = 309
Score = 290 bits (743), Expect = 1e-77
Identities = 161/306 (53%), Positives = 206/306 (67%), Gaps = 2/306 (0%)

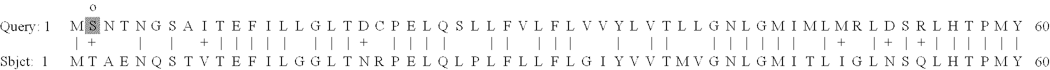

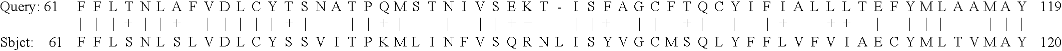

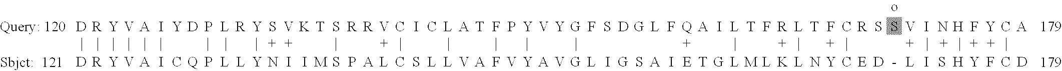

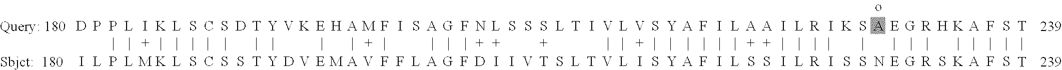

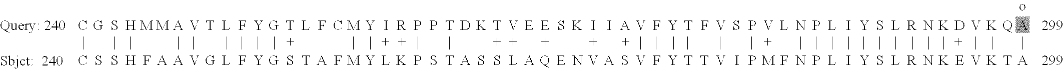

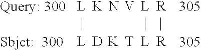

A multiple sequence alignment is given in Table 5L, with GPCR5a being shown on line 1, in a ClustalW analysis comparing GPCR5a with related protein sequences.

TABLE 5L
Information for the ClustalW proteins:

1 Novel Human_OLF, GPCR5a, SEQ ID NO:14
2. Homo sapiens OLF, SWISSPROT-ACC # Q13606, SEQ ID NO:33
3. HylobateS lar (Common Gibbon) OLF, SPTREMBL-Acc # O77758, SEQ ID NO:43
4. Rattus norvegicus OLF, SPTREMBL-ACC # Q63394, SEQ ID NO:55

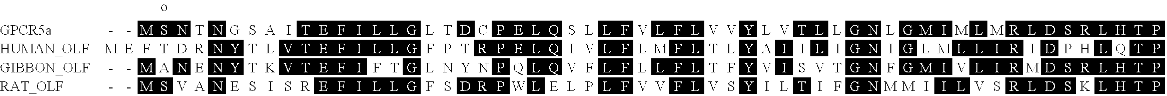

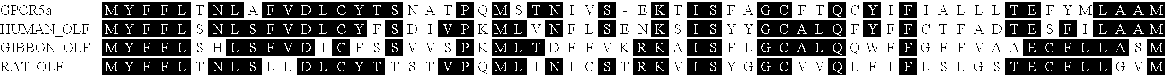

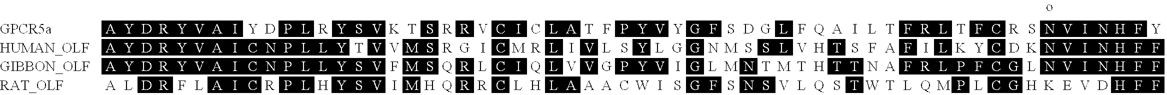

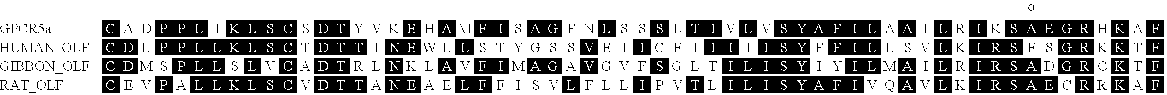

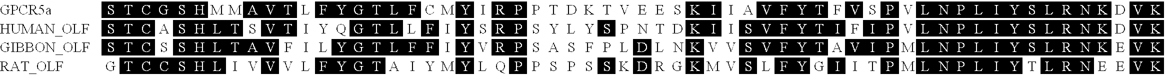

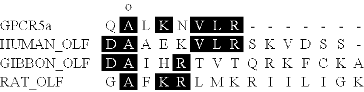

The presence of identifiable domains in GPCR5 was determined by searches using algorithms such as PROSITE, Blocks, Pfam, ProDomain, Prints and then determining the Interpro number by crossing the domain match (or numbers) using the Interpro website (http:www.ebi.ac.uk/interpro/). The results indicate that this protein contains the following protein domains (as defined by Interpro) at the indicated positions: domain name 7tm_{—}1 (InterPro) 7 transmembrane receptor (rhodopsin family) at amino acid positions 41 to 289. This indicates that the sequence of GPCR5 has properties similar to those of other proteins known to contain this/these domain(s) and similar to the properties of these domains.

DOMAIN results for GPCR5a were collected from the Conserved Domain Database (CDD) with Reverse Position Specific BLAST. This BLAST samples domains found in the Smart and Pfam collections. The results are listed in Table 5M with the statistics and domain description. Residues 1-180 (SEQ ID NO:29) and residues 313-377 (SEQ ID NO:37) of 7tm_{—}1 are aligned with GPCR4 in Table 40. The residue that differs between GPCR5a, GPCR5b and GPCR5c are highlighted in black and marked with the (o) symbol.

TABLE 5M
DOMAIN results for GPCR5a.

Sbjct: 7 transmembrane receptor (rhodopsin family) fragment (SEQ ID NO: 29)
gnl|Pfam|pfam00001, 7tm_1, 7 transmembrane receptor (rhodopsin family), 377 aa
Statistics for GPCRSa: Score = 92.8 bits (229), Expect = 2e-20
Statistics for GPCRSb: Score = 92.8 bits (229), Expect = 2e-20

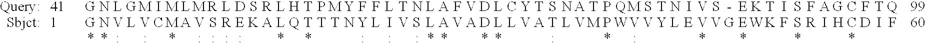

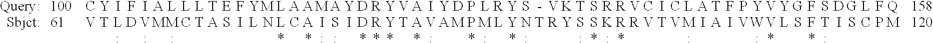

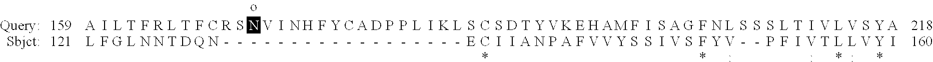

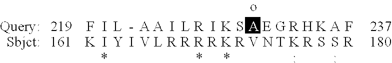

Sbjct: 7 transmembrane receptor (rhodopsin family) fragment (SEQ ID NO: 37)
gnl|Pfam|pfam00001, 7tm_1, 7 transmembrane receptor (rhodopsin family), 377 aa
Statistics for GPCR5a: Score = 35.8 bits (81), Expect = 0.003
Statistics for GPCR5b: Score = 35.8 bits (81), Expect = 0.003

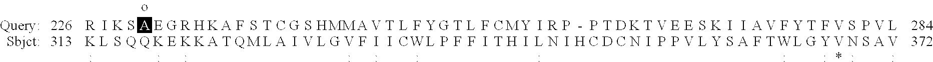

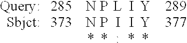

GPCR5 is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to Public EST sources, Literature sources, and/or RACE sources.

In the following positions, one or more consensus positions of GPCR5 have been identified as single nucleotide polymorphisms (“SNPs”). As shown in Table 5N, “Depth” represents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

TABLE 5N
GPCR5 Single Nucleotide Polymorphisms

| Consensus Position | Depth | Change | Putative Allele Freq |
|---|---|---|---|
| 82 | 23 | T > A | 0.478 |
| 587 | 30 | T > C | 0.067 |
| 597 | 32 | G > A | 0.375 |

The protein similarity information, expression pattern, and map location for the GPCR5 protein and nucleic acid disclosed herein suggest that GPCR5 may have important structural and/or physiological functions characteristic of the Olfactory Receptor family and the GPCR family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various GPCR- or OR-related diseases and disorders described below and/or other pathologies. For example, the compositions of the present invention will have efficacy for treatment of patients suffering from: developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy (DRPLA) Hypophosphatemic rickets, autosomal dominant (2) Acrocallosal syndrome and dyskinesias, such as Huntington's disease or Gilles de la Tourette syndrome and/or other pathologies and disorders of the like. The polypeptides can be used as immunogens to produce antibodies specific for the invention, and as vaccines. They can also be used to screen for potential agonist and antagonist compounds. For example, a cDNA encoding GPCR5 may be useful in gene therapy, and GPCR5 may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The novel nucleic acid encoding GPCR5, and the GPCR5 protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. Other GPCR-related diseases and disorders are contemplated.

These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-GPCRX Antibodies” section below. In one embodiment, a contemplated epitope of GPCR5c would be from amino acid 10 to 50. In another embodiment, a contemplated epitope of GPCR5c would be from amino acid 35 to 45. In yet another embodiment, a contemplated epitope of GPCR5c would be from amino acid 80 to 120. In yet another embodiment, a contemplated epitope of GPCR5c would be from amino acid 135 to 160. In yet another embodiment, a contemplated epitope of GPCR5c would be from amino acid 205 to 235. In yet another embodiment, a contemplated epitope of GPCR5c would be from amino acid 245 to 260. In yet another embodiment, a contemplated epitope of GPCR5c would be from amino acid 275 to 290.

GPCR6

The novel nucleic acid of 1050 nucleotides GPCR6 (also designated AP00111 2₁₃ D) encoding a novel Olfactory Receptor-like protein is shown in Table 6A. An ORF was identified beginning with an ATG initiation codon at nucleotides 53-55 and ending with a TAA codon at nucleotides 1007-1009. A putative untranslated region upstream from the initiation codon and downstream from the termination codon is underlined in Table 6A, and the start and stop codons are in bold letters.

TABLE 6A
GPCR6 Nucleotide Sequence (SEQ ID NO:19)

TGTAATGGACTTCTCATTCACCTTGATTTATTTTCATCCATTTAAGTGAAAAATGTTGGTACCTAAGAAAATGGTTAGAGGAA
ATTCTACTTTGGTGACGGAATTTATTCTCTTGGGATTAAAGGATCTTCCAGAGCTTCAGCCCATCCTCTTTGTACTGTTCCTG
CTAATCTACCTGATCACTGTCGGGGGGAACCTTGGGATGTTGGTGTTGATCAGGATAGATTCACGCCTCCACACCCCCATGTA
TTTCTTTCTTGCTAGTTTGTCCTGCTTGGATTTGTATTACTCCACTAATGTGACTCCCAAGATGTTGGTGAACTTCTTCTCAG
ACAAGAAAGCCATTTCCTATGCTGCTTGTTTAGTCCAGTGCTATTTTTTCATTGCTGTGGTGATTACTGAATATTATATGCTA
GCTGTAATGGCCTATGATAGGTATGTGGCCATCTGTAACCCTTTGCTTTACAGCAGCAAGATGTCCAAAGGGCTCTGTATTCG
CCTGATTGCTGGTCCATATGTCTATGGGTTTCTTAGTGGACTGATGGAAACCATGTGGACATACCACTTGACCTTCTGTGGCT
CCAATATCATTAATCACTTCTACTGTGCTGACCCACCCCTCATCCGACTTTCCTGCTCTGACACTTTCATTAAGGAAACATCC
ATGTTTGTGGTAGCATGTTTTAACCTCTCCAGCTCCCTCATCATAATCCTCATCTCCTACATCTTCATTCTCATTGCCATCCT
GAGGATGCGTTCTGCTGAAAGTAGGCGCAAAGCGTTCTCCACCTGCGGGTCCCACCTGGTGGCAGTGACTGTGTTTTATGGAA
CCCTGTTCTGCATGTACGTTAGACCTCCCACGGACAGGTCAGTGGAACAGTCCAAAGTCATTGCTGTTTTCTACACTTTTGTA
AGCCCTATGTTGAACCCCATCATCTATAGTTTGAGGAACAAGGATGTGAAACAAGCTTTTTGGAAACTGATCAGAAGAAACGT
GCTTTTGAAGTAAAATCAGTGTATCTTTATTAGTCAAATAAAAAAATCTTTCTA

T—sequence change from A to T to correct a stop codon.

The GPCR6 protein encoded by SEQ ID NO: 19 has 318 amino acid residues and is presented using the one-letter code in Table 6B.

TABLE 6B
Encoded GPCR6 protein sequence (SEQ ID NO:20).

MLVPKKMVRGNSTLVTEFILLGLKDLPELQPILFVLFLLIYLITVGGNLGMLVLIRIDSRLHTPMYFFLASLSCLDLYYSTNV
TPKMLVNFFSDKKAISYAACLVQCYFFIAVVITEYYMLAVMAYDRYVAICNPLLYSSKMSKGLCIRLIAGPYVYGFLSGLMET
MWTYHLTFCGSNIINHFYCADPPLIRLSCSDTFIKETSMFVVACFNLSSSLIIILISYIFILIAILRMRSAESRRKAFSTCGS
HLVAVTVFYGTLFCMYVRPPTDRSVEQSKVIAVFYTFVSPMLNPIIYSLRNKDVKQAFWKLIRRNVLLK

In a search of sequence databases, it was found, for example, that the GPCR6 nucleic acid sequence has 621 of 939 bases (66%) identical to a and 621 of 939 bases (66%) positive with Gallus gallus species Olfactory Receptor 2 (GENBANK-ID: X94742) (Table 6C).

TABLE 6C
BLASTN of GPCR6 against OR2 (SEQ ID NO:51)

>gb:GENBANK-ID:GCCOR2GEN|acc:X94742 G. gallus cor2 DNA for
olfactory receptor 2 Gallus gallus, (SEQ ID NO:51) 996 bp.
Score = 1539 (230.9 bits), Expect = 1.8e-63, P = 1.8e-63
Identities = 621/939 (66%), Positives = 621/939 (66%), Strand = Plus/Plus

| | | |
|---|---|---|
| Query: | 67 GAAAATGGTTAGAGGAAATTCTACTTTGGTGACGGAATTTATTCTCTTGGGATTAAAGGA | 126 |
| | \|\| \|\|\|\| \| \|\|\|\|\|\| \| \| \| \|\| \|\|\|\|\|\| \| \|\|\|\|\|\|\|\|\|\|\| \|\| | |
| Sbjct: | 54 GATGATGGCCAAGGGAAATCACAGCTCCATCACTGAATTTGTGCTCTTGGGATTCTCTGA | 113 |
| Query: | 127 TCTTCCAGAGCTTCAGCCCATCCTCTTTGTACTGTTCCTGCTAATCTACCTGATCACTGT | 186 |
| | \| \| \|\|\| \| \| \|\|\|\|\|\| \| \|\|\| \|\|\|\| \|\|\|\|\|\|\|\|\|\|\|\|\|\|\| \| | |
| Sbjct: | 114 AAAGAGGGCCATCCAGGCTGTTCTCTTTATGGGCTTCTTGCTGATCTACCTCATCACTCT | 173 |
| Query: | 187 CGGGGGGAACCTTGGGATGTTGGTGTTGATCAGGATAGATTCACGCCTCCACACCCCCAT | 246 |
| | \|\| \|\| \| \|\| \|\|\| \| \|\|\|\|\|\|\|\|\| \| \|\| \|\| \|\| \|\| \|\|\|\|\|\|\|\| \|\| | |
| Sbjct: | 174 GCTAGGCAATGTGGGCATGATCACATTGATCAGGCTGGACTCCCGGCTTCACACCCCTAT | 233 |
| Query: | 247 GTATTTCTTTCTT-GCTAGTTTGTCCTGCTTGGAT-T-TG-TATTACTCCACTAATGTGA | 302 |
| | \|\|\| \|\|\|\|\| \|\| \|\| \|\| \|\|\|\|\|\|\| \| \| \|\|\| \| \|\| \|\|\|\| \|\|\|\|\|\| \|\|\| \| | |
| Sbjct: | 234 GTACTTCTTCCTGAGC-AGCTTGTCCTTCCTCGATATCTGCTATTCCTCCAC-AATC--A | 289 |
| Query: | 303 CTCC-CAAGATGTTGGTGA-ACTTCTTCTCAGACA-AGAAAGCCATTTCCTATGCTGCTT | 359 |
| | \|\|\|\| \| \|\| \|\| \| \| \| \|\| \|\| \|\| \|\| \|\|\|\|\|\| \|\|\|\|\|\|\| \| \|\|\|\| \| | |
| Sbjct: | 290 CTCCTCGAG-TGCTC-TCAGACCTCCCAGCAT-CACAGAAAGTCATTTCCCACTCTGCAT | 346 |
| Query: | 360 GTTTAGTCCAGTGCTATTTTTTCATTG-CTGTGGTGATTACTGAATATTATATGCTAGCT | 418 |
| | \| \| \| \|\|\|\| \|\|\|\|\| \| \| \|\| \|\| \|\| \| \|\| \|\| \| \|\|\| \| \| \|\| | |
| Sbjct: | 347 GCCTGGCACAGTTTTATTTCTACGCTGTCTTTGCC-ACCACAGAGTGCTATCTTTTGGCC | 405 |
| Query: | 419 GTAATGGCCTATGATAGGTATGTGGCCATCTGTAACCCTTTGCTTTACAGCAGCAAGATG | 478 |
| | \| \|\|\|\|\|\|\|\|\|\|\| \| \|\| \|\|\|\|\|\|\|\|\|\|\| \| \|\|\|\| \|\|\|\| \|\| \| \| \|\|\| | |
| Sbjct: | 406 GCAATGGCATATGACCGCTACGTGGCCATCTGCAGCCCTCTGCTCTATGTCTTCTCCATG | 465 |
| Query: | 479 TCCAA-AGGGCTCTGTATTCGCCTGATTGCTGGTCCATATGTCTAT-GGGTTTCTTAGTG | 536 |
| | \|\|\|\| \|\| \| \| \|\|\| \| \| \|\|\| \|\|\|\|\|\|\| \|\|\|\| \| \| \| \|\|\| \|\| \| \| \|\| | |
| Sbjct: | 466 TCCAGCAGAGTT-TGTGTGCTGCTGGTTGCTGGCTCATACCT-TGTCGGGGTTGTGAATG | 523 |
| Query: | 537 G-ACTGATGGAAACCATG-TGGACATACCACTTGACCTTCTGTGGCTCCAATATCATTAA | 594 |
| | \|\| \|\| \| \|\| \| \| \| \|\| \|\| \|\| \|\|\|\|\|\|\|\|\|\| \|\|\|\| \|\|\|\|\| \|\| | |
| Sbjct: | 524 CCACC-ATTCACACAGGGCTTG-CACTGCAGCTGTCCTTCTGTGGTCCCAACATCATCAA | 581 |
| Query: | 595 TCACTTCTACTGTG-CTGACCCACCCCTCATCCGACTT-TCCTGCTCTGACACTTTCATT | 652 |
| | \|\|\|\|\|\|\|\|\|\|\|\|\|\| \| \| \|\|\| \|\| \|\|\| \| \|\| \| \| \|\| \|\|\| \| \|\|\| \| \|\| | |
| Sbjct: | 582 TCACTTCTACTGTGACGGTCCC-CCGCTC-TACGCCATCTCGTGCACAGACCCCACCACC | 639 |
| Query: | 653 AAGGAAACATCCATGTTTGTGGTAGCATGATTTAACCT-CTCCAGCTCCCTCATCATAAT | 711 |
| | \|\| \|\|\| \| \|\| \|\|\| \| \|\| \| \| \|\| \|\|\| \| \|\|\| \| \| \|\| \|\|\| \|\| | |
| Sbjct: | 640 AACGAGATTGCGATATTTCTTGTGGTTGGCTTCAACATGCTC-ATCACCAGCGTGACCAT | 698 |
| Query: | 712 CCTCATCTCCTACATCTTCATTCTCATTGCCATCCTGAGGATGCGTTCTGCTGAAAGTAG | 771 |
| | \| \|\|\|\|\|\|\|\|\|\|\|\| \|\| \|\|\| \|\| \| \|\| \|\|\|\| \|\|\|\|\|\|\| \| \|\|\|\| \| \| \| | |
| Sbjct: | 699 CTTCATCTCCTACACCTACATCCTGTTCGCTGTCCTCAGGATGCACACAGCTGCAGGCAA | 758 |
| Query: | 772 GCGCAAAGCGTTCTCCACCTGCGGGTCCCACCTGGTGGCAGTGACTGTGTTTTATGGAAC | 831 |
| | \|\|\|\|\|\| \| \|\|\|\|\|\|\|\| \|\| \| \|\|\|\|\|\|\|\|\|\|\| \| \|\| \|\| \| \|\| \|\|\|\| \| | |
| Sbjct: | 759 ACGCAAAACCTTCTCCACGTGTGCGTCCCACCTGGCCACCGTCACCCTATTCTATGCCTC | 818 |
| Query: | 832 C-CTGTTCTGCATGTACGTTA--GACCTCCCACGGACAGGTCAGTGGAACAGTCCAAAGT | 888 |
| | \|\|\| \| \| \|\|\|\|\|\|\| \| \| \| \|\| \| \| \| \|\| \|\| \|\|\| \| \| \|\|\| \|\| | |
| Sbjct: | 819 TGCTGGT-TCCATGTAC-TCACGGCCCAGCTCCAGGCAC-TCCCAGGACCTGGACAAGGT | 875 |
| Query: | 889 CATTGCTGTTTTCTACACTTTTGTAAGCCCTATGTTGAACCCCATCATCTATAGTTTGAG | 948 |
| | \|\|\|\| \|\|\|\|\|\|\|\| \| \|\| \| \|\|\| \|\|\| \|\|\|\|\|\|\|\| \|\|\|\|\|\|\| \|\| \|\|\|\| | |
| Sbjct: | 876 GGCCTGTGTATTCTACACCATGGTGACCCCCATGCTGAACCCCCTCATCTACAGCCTGAG | 935 |
| Query: | 949 GAACAAGGATGTGLAACAAGCTTTTTGGAAACTGATCAGAAGAAAC-GTGCTTTTGA | 1004 |
| | \|\|\|\| \|\|\|\| \|\| \|\| \| \| \|\|\| \|\|\|\|\| \|\|\|\| \| \|\| \|\| \|\|\| \| \|\|\| | |
| Sbjct: | 936 GAACCAGGAGGTAAAGGATGTTTTAGGGAAAGTGATGGGGAGGAAGAGTGTCTCTGA | 992 |

The GPCR6 amino acid has 165 of 307 amino acid residues (53%) identical to, and 226 of 307 residues (73%) positive with, the 312 amino acid OR4 from Gallus gallus (ptnr: SPTREMBL-ACC: 077756) (SEQ ID NO:56) (Table 6D).

TABLE 6D
BLASTX of GPCR6 against OR4

>ptnr:SPTREMBL-ACC:Q90808 OLFACTORY RECEPTOR 4 - Callus gallus
(Chicken), 312 aa (fragment). (SEQ ID NO:56)
Score = 867 (305.2 bits), Expect = 7.0e-86, P = 7.0e-86
Identities = 165/307 (53%), Positives = 226/307 (73%), Frame = +2

| | | |
|---|---|---|
| Query: | 71 MVRGNSTLVTEFILLGLKDLPELQPILFVLFLLIYLITVGGNLGMLVLIRIDSRLHTPMY | 250 |
| | \| \|\| \|\| +\|\|\|\|+\|\| \| \|+++ \|\|\|+\|\|\|\|\|+\|\| \|\|\|\|+++\|\|+ \| \|\|\|\| \|\| | |
| Sbjct: | 1 MAEGNHTLASEFILVGLSDRPKMKAALFVVFLLIYVITFQGNLGIIILIQGDPRLHTSMY | 60 |
| Query: | 251 FFLASLSCLDLYYSTNVTPKMLVNFFSDKKAISYAACLVQCYFFIAVVITEYYMLAVMAY | 430 |
| | \|\|\|+\|\|\| +\|+ +\|+ + \|+ \|\|\|\| \|+++ \|\|+ \| \| +\|+\| \| \|\| ++\|\|\|\|\|\| | |
| Sbjct: | 61 FFLSSLSVVDICFSSVIAPRTLVNFLSERRTISFTGCTCQTFFYIVFVTTECFLLAVMAY | 120 |
| Query: | 431 DRYVAICNPLLYSSKMSKGLCIRLIAGPYVYGFLSGLMETMWTYHLTFCGSNIINHFYCA | 610 |
| | \|\|\|\|\|\|\|\|\|\|\|\|\|+ \|++ \|++\|+ \| \|+ \| \|+ +++\| + \| \|\|\|\|\|\|\|\|\|\|+\| | |
| Sbjct: | 121 DRYVAICNPLLYSTIMTRRQCMQLVVGSYIGGILNAIIQTTFIIRLPFCCSNIINHFFCD | 180 |
| Query: | 611 DPPLIRLSCSDTFIKETSMFVVA*FNLSSSLIIILISYIFILIAILRMRSAESRRKAFST | 790 |
| | \|\|\|+ \|\| + \|+\| \| +\| +\| \|++ \|\|+\|\|\|\|\| \|\|\|\|+\|\|\|\| \|+\|\| \|\| | |
| Sbjct: | 181 VPPLLALSLASTYISEMILFSLAGIIELSTVTSILVSYIFISCAILRIRSAEGRQKALST | 240 |
| Query: | 791 CGSHLVAVTVFYGTLFCMYVRPPTDRSVEQSKVIAVFYTFVSPMLNPIIYSLRNKDVRQA | 970 |
| | \| \|\|\| \|\|\|+ \|\|\| \|+\|\| + \|+ \|\|++\|\|\|\| \| \|\|\|\|\|+\|\|\|\|\|\|++\|\| \| | |
| Sbjct: | 241 CASHLTAVTLLYGTTIFTYLRPSSSYSLNTDKVVSVFYTVVIPMLNPLIYSLRNQEVKGA | 300 |
| Query: | 971 FWKLIRR | 991 |
| | +++ \| | |
| Sbjct: | 301 LSRVVER | 307 |

The GPCR6 amino acid has 153 of 313 amino acid residues (48%) identical to, and 199 of 313 residues (62%) positive with, the 314 amino acid OR93Ch from Pan troglodytes OR93Ch (GENBANK-ID:AAC63969.1) (SEQ ID NO:57) (Table 6E).

TABLE 6E
BLASTX of GPCR6 against OR93Ch

>gb|AAC63969.1| (AF045577) olfactory receptor OR93Ch
[Pan troglodytes] (SEQ ID NO:57) Length = 314
Score = 293 bits (749), Expect = 2e-78
Identities = 169/313 (54%), Positives = 215/313 (69%),
Gaps = 1/313 (0%)

| | | |
|---|---|---|
| Query: 7 | MVRGNSTLVTEFILLGLKDLPELQPILFVLFLLIYLI-TVGGNLGMLVLIRIDSRLHTPM | 65 |
| | \| \| \| \|\|\|\|\| \|\| \|+\|\| \|\| \|\|\| \| \|\|\|\|\|+\|\|\|\|\|\|\|\|\|\|\|\|\| | |
| Sbjct: 1 | MANENYTKVTEFIFTGLNYNPQLQVFLFLLFLTTFYVINVTGNLGMIVLIRIDSRLHTPM | 60 |
| Query: 66 | YFFLASLSCLDLYYSTNVTPKMLVNFFSDKKAISYAACLVQCYFFIAVVITEYYMLAVMA | 125 |
| | \|\|\|\|+ \|\| +\|+ +\|+ \|+\|\|\|\| +\|\| +\|\|\|\|+ \| +\| +\|\| \| \| ++\|\| \|\| | |
| Sbjct: 61 | YFFLSHLSFVDICFSSVVSPKMLTDFFVKRKAISFLGCALQQWFFGFFVAAECFLLASMA | 120 |
| Query: 126 | YDRYVAICNPLLYSSKNSKGLCIRLIAGPYVYGFLSGLMETMWTYHLTFCGSNIINHFYC | 185 |
| | \|\|\|\|\|\|\|\|\|\|\|\|\|\| \|\|+ \|\|\|+\|+ \|\|\|\| \| ++ + \| + \| \|\|\| \|+\|\|\|\|+\| | |
| Sbjct: 121 | YDRYVAICNPLLYSVANSQRLCIQLVVGPYVIGLMNTMTHTTNAFRLPECGPNVINHFFC | 180 |
| Query: 186 | ADPPLIRLSCSDTFIKETSMFVVACFNLSSSLIIILISYIFILIAILRMRSAESRRKAFS | 245 |
| | \|\|+ \| \|+\|\| + + ++\|+\|\| \| \|\|\|\|\|\| \|\| \|\|\|\|+\|\|\|+ \| \| \|\| | |
| Sbjct: 181 | DMSPLLSLVCADTRLNKLAVFIVAGAAGVFSGLTILISYIYILMAILRIRSADGRCKTFS | 240 |
| Query: 246 | TCGSELVAVTVFYGTLFCMYVRPPTDRSVEQSKVIAVFYTFVSPMLNPIIYSLRNKDVKQ | 305 |
| | \|\| \|\|\| \|\| + \|\|\|\|\| +\|\|\|\| \|++ +\|+++\|\|\|\| \| \|\|\|\|\|+\|\|\|\|\|\|\|+\|\| | |
| Sbjct: 241 | TCSSHLTAVFILYGTLFFIYVRPSASFSLDLNKLVSVFYTAVIPMLNPLIYSLRNKEVKD | 300 |
| Query: 306 | AFWKLIRRNVLLK | 318 |
| | \| + + + \| | |
| Sbjct: 301 | AIHRTVTQRKFCK | 313 |

The GPCR6 amino acid has 150 of 312 amino acid residues (48%) identical to, and 198 of 312 residues (63%) positive with, the 313 amino acid OR93Gib from Hylobates lar (GENBANK-ID:AAC63971.1) (SEQ ID NO:58) (Table 6F).

TABLE 6F
BLASTX of GPCR6 against OR93Gib

>gb|AAC63971.1| (AF045580) olfactory receptor OR93Gib [Hylobates lar]
(SEQ ID NO:58) Length = 313
Score = 291 bits (745), Expect = 7e-78
Identities = 168/312 (54%), Positives = 216/312 (69%)

| | | |
|---|---|---|
| Query: 7 | MVRGNSTLVTEFILLGLKDLPELQPILFVLFLLIYLITVGGNLGMLVLIRIDSRLHTPMY | 66 |
| | \| \| \| \|\|\|\|\| \|\| \|+\|\| \|\| \|\|\| \| \|+\| \|\| \|\|+\|\|\|\|+\|\|\|\|\|\|\|\|\| | |
| Sbjct: 1 | MANENYTKVTEFIFTGLNYNPQLQVFLFLLFLTFYVISVTGNFGMIVLIRMDSRLHTPMY | 60 |
| Query: 67 | FFLASLSCLDLYYSTNVTPKMLVNFFSDKKAISYAACLVQCYFFIAVVITEYYMLAVMAY | 126 |
| | \|\|\|+ \|\| +\|+ +\|+ \|+\|\|\|\| +\|\| +\|\|\|\|+ \| +\| +\|\| \| \| ++\|\| \|\|\| | |
| Sbjct: 61 | FFLSHLSFVDICFSSVVSPKMLTDFFVKRKAISFLGCALQQWFFGFFVAAECFLLASMAY | 120 |
| Query: 127 | DRYVAICNPLLYSSKMSKGLCIRLIAGPYVYGFLSGLMETMWTYHLTFCGSNIINHFYCA | 186 |
| | \|\|\|\|\|\|\|\|\|\|\|\|\| \|\|+ \|\|\|+\|+ \|\|\|\| \| ++ + \| + \| \|\|\| \|+\|\|\|\|+\| | |
| Sbjct: 121 | DRYVAICNPLLYSVFMSQRLCIQLVVGPYVIGLMNTMTHTTNAFRLPFCGLNVINHFFCD | 180 |
| Query: 187 | DPPLIRLSCSDTFIKETSMFVVACFNLSSSLIIILISYIFILIAILRMRSAESRRKAFST | 246 |
| | \|\|+ \| \|+\|\| + + ++\|++\| \| \|\|\|\|\| \|\| \|\|\|\|+\|\|\|+ \| \| \|\|\| | |
| Sbjct: 181 | MSPLLSLVCADTRLNKLAVFIMAGAVGVFSGLTILISYIYILMAILRIRSADGRCKTFST | 240 |
| Query: 247 | CGSHLVAVTVFYGTLFCMYVRPPTDRSVEQSKVIAVFYTFVSPMLNPIIYSLRNKDVKQA | 306 |
| | \| \|\|\| \|\| + \|\|\|\|\| +\|\|\|\| ++ +\|\|++\|\|\|\| \| \|\|\|\|\|+\|\|\|\|\|\|\|+\|\| \| | |
| Sbjct: 241 | CSSHLTAVFILYGTLFFIYVRPSASFPLDLNKVVSVFYTAVIPMLNPLIYSLRNKEVKDA | 300 |
| Query: 307 | FWKLIRRNVLLK | 318 |
| | + + + \| | |
| Sbjct: 301 | IHRTVTQRKFCK | 312 |

The GPCR6 amino acid has 143 of 307 amino acid residues (46%) identical to, and 193 of 307 residues (62%) positive with, the 332 amino acid OR2 from Gallus gallus (embCAA64368.1) (SEQ ID NO:59) (Table 6G).

TABLE 6G
BLASTX of GPCR6 against OR2

```
>emb |CAA64368.1 | (X94742) olfactory receptor 2 [GaIlus gallus](SEQ ID NO:59), 332
aa
Score =290 bits (743), Expect =le-77
Identities =160/307 (52%), Positives =210/307 (68%)
Query: 7 MVRGNSTLVTEFILLGLKDLPELQPILFVLFLLIYLITVGGNLGMLVLIRIDSRLHTPMY 66
                        | +|| + +|||+||| + +| +||||||||||+||+||+|||+|||||||||
Sbjct: 20 MAKGNHSSITEFLLLGFSEKRAIQAVLFMGFLLIYLITLLGNVGMITLIRLDSRLHTPMY 79
Query: 67 FFLASLSCLDLYYSTNVTPKMLVNFFSDKKAISYAACLVQCYFFIAVVITEYYMLAVMAY 126
                        |||+|||||+||++||++|+++|||++||||||+|||+|||||
Sbjct: 80 FFLSSLSFLDICYSSTITPRVLSDLPASQKVISHSACLAQFYFYAVFATTECYLLAAMAY 139
Query: 127 DRYVAICNPLLYSSKMSKGLCIRLIAGPYVYGFLSGLMETMWTYHLTFCGSNIINHFYCA 186
                        |||||||+||||||+|+|+|||+|+++||+|||||||||||
Sbjct: 140 DRYVAICSPLLYVFSMSSRVCVLLVAGSYLVGVVNATIHTGLALQLSFCGPNIINHFYCD 199
Query: 187 DPPLIRLSCSDTFIKETSMFVVACFNLSSSLIIILISYIFILIAILRMRSAESRRKAFST 246
                          |||+||+||++|+||||||||||||||+|+|||||
Sbjct: 200 GPPLYAISCTDPTTNEIAIFLVVGFNMLITSVTIFISYTYILFAVLRMHTAAGKRKTFST 259
Query: 247 CGSHLVAVTVFYGTLFCMYVRPPTDRSVEQSKVIAVFYTFVSPMLNPIIYSLRNKDVKQA 306
                        |||| ||+||+||||+|+||+|||||+|||||+||||||++||
Sbjct: 260 CASHLATVTLFYASAGSMYSRPSSRHSQDLDKVASVFYTMVTPMLNPLIYSLRNQEVKDV 319
Query: 307 FWKLIRR 313
                            |++ |
Sbjct: 320 LGKVMGR 326
```

The GPCR6 amino acid has 150 of 311 amino acid residues (48%) identical to, and 193 of 311 residues (61%) positive with, the 311 amino acid K30 from Mus musculus (GENBANK-ID:AAG39871.1) (SEQ ID NO:60) (Table 6H).

TABLE 6H
BLASTX of GPCR6 against K30

```
>gb |AAG39871.1 |AF282286_1 (AF282286) odorant receptor K30 [MUS musculus]
(SEQ ID NO:60) Length = 311
Score = 290 bits (743), Expect = 1e-77
Identities = 166 311 (53%), Positives = 206 311 (66%)
Query: 7 MVRGNSTLVTEFILLGLKDLPELQPILFVLFLLIYLITVGGNLGMLVLIRIDSRLHTPMY 66
                        |++||+||||||||+||||||||||||||||||++||+|||||||
Sbjct: 1 MLKGNLSEVTEFILAGLTNKPELQLPLFLLFLAIYVVTVVGNLGMIILILLSSHLHTPMY 60
Query: 67 FFLASLSCLDLYYSTNVTPKMLVNFFSDKKAISYAACLVQCYFFIAVVITEYYMLAVMAY 126
                        +||+|||+||||+|||||||+|||||+||||+||+|||||||
Sbjct: 61 YFLSSLSFIDLCQSTVIIPKMLVNFVTVKNIISYPECMTQLYFFVTFAIAECHMLAVMAY 120
Query: 127 DRYVAICNPLLYSSKMSKGLCIRLIAGPYVYGFLSGLMETMWTYHLTFCGSNIINHFYCA 186
                        ||||||||||||++||+|+|||+|++||+|+|||++|
Sbjct: 121 DRYVAICNPLLYNAVMSFQVCSSMIFGVYSIALIGATTHTVCMLRVNFCKANVINHYFCD 180
Query: 187 DPPLIRLSCSDTFIKETSMFVVACFNLSSSLIIIIISYIFILIAILRMRSAESRRKAFST 246
                            ||+||||||||+|||||||||||+++||||||||
Sbjct: 181 LFPLLELPCSDTFINEVVVLCFSVFNIFIPTLTILTSYIFIIASILQIKSTEGRSKAFST 240
Query: 247 CGSHLVAVTVFYGTLFCMYVRPPTDRSVEQSKVIAVFYTFVSPMLNPIIYSLRNKDVKQA 306
                        |||+||+|+|+|||++|+|++|||+||||||||||+|||||||||||
Sbjct: 241 CSSHISAVAIFFGSLAFMYLQPSSVSSMDQGKVSSVFYTIVVPMLNPLIYSLRNKDVKVA 300
Query: 307 FWKLIRRNVLL 317
                            |+| | +
Sbjct: 301 LNKFFERKFFL 311
```

The GPCR6 amino acid has 149 of 311 amino acid residues (47%) identical to, and 192 of 311 residues (60%) positive with, the 314 amino acid K11 from Mus musculus (GENBANK-ID:AAG39856.1) (SEQ ID NO:61) (Table 61).

TABLE 6I
BLASTX of GPCR6 against K11 (SEQ ID NO:61)

>gb|AAG39856.1|AF282271_1 (AF282271) odorant receptor K11
[Mus musculus] (SEQ ID NO:61), 314 aa
Score = 289 bits (739), Expect = 4e−77
Identities = 164/311 (53%), positives = 207/311 (66%)

| | | | |
|---|---|---|---|
| Query: | 7 | MVRGNSTLVTEFILLGLKDLPELQPILFVLFLLIYLITVGGNLGNLVLIRIDSRLETPMY | 66 |
| | | \| \|\| \|\|\|\| \| \|\| + \|\|\|\| \|\| \|\| \|\| \|\|\| \|\|\|\|\|++\|\| + \| \|\|\|\|\|\| | |
| Sbjct: | 4 | MTSGNYCTVTEFFLAGLSEKPELQLPLFFLFIGIYMITVAGNLGMIILIGLSSHLETPMY | 63 |
| Query: | 67 | FFLASLSCLDLYYSTNVTPKMLVNFFSDKKAISYAACLVQCYFFIAVVITEYYMLAVMAY | 126 |
| | | +\|\|+\|\|\| +\| \|\| \|\|\|\|\|\|\|\|\| ++\| \|\|\| \|+ \| \|\|\|+ \| \| \|+\|\| \|\|\| | |
| Sbjct: | 64 | YFLSSLSFIDFCQSTVVTPKMLVNFVTEKNIISYPGCMTQLYFFLIFAIAECYILAAMAY | 123 |
| Query: | 127 | DRYVAICNPLLYSSKMSKGLCIRLIAGPYVYGFLSGLMETMWTYHLTFCGSNIINHFYCA | 186 |
| | | \|\|\|\|\|\|\|\|\|\|\|\|+ \|\| + \| \|\|+\| \|+ \| + \| + + \|\| ++\|\|\|++\| | |
| Sbjct: | 124 | DRYVAICNPLLYNVTMSYQIYIFLISGVYIIGVICASAHTGFMVRIRFCKLDVINHYFCD | 183 |
| Query: | 187 | DPPLIRLSCSDTFIKETSMFVVACFNLSSSLIIILISYIFILIAILRMRSAESRRKAFST | 246 |
| | | \|\|++\|+\|\|+\|+\| \| + \| \| \|\|\|\|\| \|\|\|+\|\| \| \| \|\|\|\|\| | |
| Sbjct: | 184 | LLPLLKLACSNTYINEMLILFFGTLNIFVPILTIITSYIFIIASILRIRSTEGRSKAFST | 243 |
| Query: | 247 | CGSHLVAVTVFYGTLFCMYVRPPTDRSVEQSKVIAVFYTFVSPMLNPIIYSLRNKDVKQA | 306 |
| | | \| \|\|++\|\| \|\|+\|+\| \|\|++\| + \|++\| \|\| +\|\|\|\| \| \|\|\|\|\|+\|\|\|\|\|\|\|\|\| \| | |
| Sbjct: | 244 | CSSHILAVAVFFGSLAFMYLQPSSVSSMDQGKVSSVFYTIVVPMLNPLIYSLRNKDVAVA | 303 |
| Query: | 307 | FWKLIRRNVLL | 317 |
| | | \|+\| \| + | |
| Sbjct: | 304 | LKKIIERKTFM | 314 |

A multiple sequence alignment is given in Table 6J, with the GPCR6 protein being shown on line 4, in a ClustalW analysis comparing GPCR6 with related protein sequences.

TABLE 6J
Information for the ClustalW proteins:

1. Gallus gallus (CHICKEN) OLF, SPTREMBL-ACC # Q90808, SEQ ID NO:56
2. Hylobates lar (Common Gibbon) OLF, SPTREMBL-Acc # O77758, SEQ ID NO:43
3. Homo sapiens OLF, SWISSPROT-Acc # Q13606, SEQ ID NO:33
4. Novel Human_OLF, GPCR6, SEQ ID NO:20
5. Rattus norvegicus OR, Acc # G264617, SEQ ID NO:62

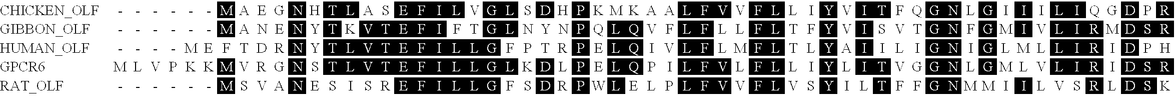

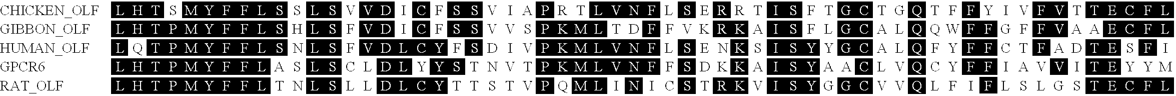

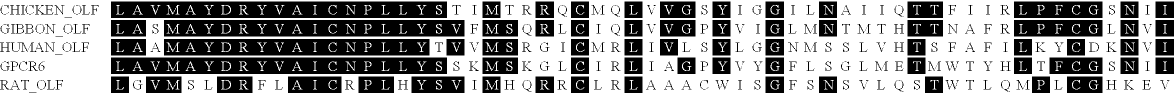

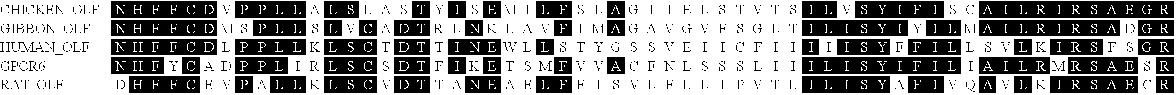

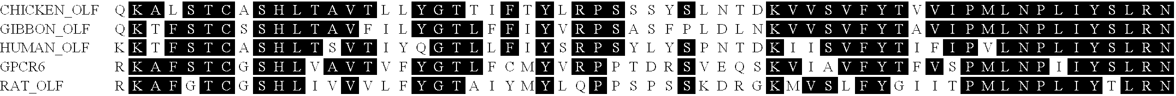

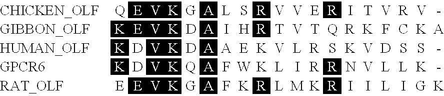

DOMAIN results for GPCR6 were collected from the Conserved Domain Database (CDD) with Reverse Position Specific BLAST. This BLAST samples domains found in the Smart and Pfam collections. The results are listed in Table 6K with the statistics and domain description. Residues 1-158 (SEQ ID NO:29) and residues 313-377 (SEQ ID NO:37) of 7tm_{—}1 are aligned with GPCR4 in Table 6K.

TABLE 6K
DOMAIN results for GPCR6

Sbjct: 7 transmembrane receptor (rhodopsin family) fragment (SEQ ID NO:29)
gnl|Pfam|pfam00001, 7tm_1, 7 transmembrane receptor (rhodopsin family)

Length = 377
Score = 95.1 bits (235), Expect = 5e-21

| | | | |
|---|---|---|---|
| Query: | 47 | GNLGMLVLIRIDSRLHTPMYFFLASLSCLDLYYSTNVTPKMLVNFFSDKKAISYAACLVQ | 106 |
| Sbjct: | 1 | GNVLVCMAVSREKQLQTTTNYLIVSLAVADLLVATLVMPWVVYLEVVGEWKFSRIHCDIF | 60 |
| | | **: : : : : * * : : **: ** :* * * :: : * * : | |
| Query: | 107 | CYFFIAVVITEYYMLAVMAYDRYVAICNPLLYSSKM-SKGLCIRLIAGPYVYGFLSGLME | 165 |
| Sbjct: | 61 | VTLDVMMCTASILNLCAISIDRYTAVAMPMLYNTRYSSKRRVTVMIAIVWVLSFTISCPM | 120 |
| | | : : * :: *** *: *:**::: ** :** :* * | |
| Query: | 166 | -TMWTYHLTF-CGSNIINHFYCADPPLIRLSCSDTFIKETSMFVVA | 209 |
| Sbjct: | 121 | LFGLNNTDQNECIIANPAFVVY--------SSIVSFYVPFIVTLLV | 158 |
| | | * * :* : :: | |

| | |
|---|---|
| Sbjct: | 7 transmembrane receptor (rhodopsin family) fragment (SEQ ID NO:37) |
| gnl\|Pfam\|pfam00001, 7tm_1, 7 transmembrane receptor (rhodopsin family) | |

Length = 377
Score = 38.5 bits (88), Expect = 5e-04

| | | | |
|---|---|---|---|
| Query: | 233 | RMRSAESRRKAFSTCGSHLVAVTVFYGTLFCMYVRP-PTDRSVEQSKVIAVFYTFVSPML | 291 |
| Sbjct: | 313 | KLSQQKEKKATQMLAIVLGVFIICWLPFFITHILNIHCDCNIPPVLYSAFTWLGYVNSAV | 372 |
| | | :: : :: * : : : : :*: : | |
| Query: | 292 | NPIIY | 296 |
| Sbjct: | 373 | NPIIY | 377 |
| | | ***** | |

The nucleic acids and proteins of the invention are useful in potential therapeutic applications implicated in various in various GPCR-related pathological disorders and/or OR-related pathological disorders, described further below. For example, a cDNA encoding the olfactory receptor-like protein may be useful in gene therapy, and the olfactory receptor-like protein may be useful when administered to a subject in need thereof. By way of nonlimiting example, the compositions of the present invention will have efficacy for treatment of patients suffering from Neoplasm; adenocarcinoma; lymphoma; prostate cancer; uterus cancer; Immune response; AIDS; asthma; Crohn's disease; multiple sclerosis; and Albright Hereditary Ostoeodystrophy. Other GPCR-related diseases and disorders are contemplated.

The GPCR6 nucleic acid and protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. This novel protein also has immense value in development of powerful assay system for functional analysis of various human disorders, which will help in understanding of pathology of the disease and development of new drug targets for various disorders.

GPCR7

The novel nucleic acid of 981 nucleotides GPCR7 (also designated. AP001112_dal) encoding a novel OR-like protein is shown in Table 7A. An ORF begins with an ATG initiation codon at nucleotides 27-29 and ends with a TGA codon at nucleotides 942-944. Putative untranslated regions, if any, are found upstream from the initiation codon and downstream from the termination codon.

TABLE 7A
GPCR7 Nucleotide Sequence (SEQ ID NO:21)

AGCTTGAAGAGCAAACTGTCAGGAATATGTCCAACACAAATGGCAGTGCAATCACAGAATTCATTTTACTTGGGC
TCACAGATTGCCCGGAACTCCAGTCTCTGCTTTTTGTGCTGTTTCTGGTTGTTTACCTCGTCACCCTGCTAGGCA
ACCTGGGCATGATAATGTTAATGAGACTGGACTCTCGCCTTCACACGCCCATGTACTTCTTCCTCACTAACTTAG
CCTTTGTGGATTTGTGCTATACATCAAATGCAACCCCGCAGATGTCGACTAATATCGTATCTGAGAAGACCATTT
CCTTTGCTGGTTGCTTTACACAGTGCTACATTTTCATTGCCCTTCTACTCACTGAGTTTTACATGCTGGCAGCAA
TGGCCTATGACCGCTATGTGGCCATATATGACCCTCTGCGCTACAGTGTGAAAACGTCCAGGAGAGTTTGCATCT
GCTTGGCCACATTTCCCTATGTCTATGGCTTCTCAGATGGACTCTTCCAGGCCATCCTGACCTTCCGCCTGACCT
TCTGTAGATCCAATGTCATCAACCACTTCTACTGTGCTGACCCGCCGCTCATTAAGCTTTCTTGTTCTGATACTT
ATGTCAAAGAGCATGCCATGTTCATATCTGCTGGCTTCAACCTCTCCAGCTCCCTCACCATCGTCTTGGTGTCCT
ATGCCTTCATTCTTGCTGCCATCCTCCGGATCAAATGCAGAGCGAGGGAAGGCACGGCATTCTCCACCTGTGGTT
CCCATATGATGGCTGTCACCCTGTTTTATGGGACTCTCTTTTGCATGTATATAAGACCACCAACAGATAAGACTG
TTGAGGAATCTAAAATAATAGCTGTCTTTTACACCTTTGTGAGTCCGGTACTTAATCCATTGATCTACAGTCTGA
GGAATAAAGATGTGAAGCAGGCCTTGAAGAATGTCCTGAGATGAAATATTGTCATGACCATGGTGATGCCTTTGT
TTCCTA

The GPCR7 protein encoded by SEQ ID NO:20 has 305 amino acid residues and is presented using the one-letter code in Table 7B. The SignalP, Psort and/or Hydropathy profile for GPCR7 predict that this sequence has a signal peptide and is likely to be localized at the plasma membrane with a certainty of 0.6000. The SignalP shows a cleavage site between amino acids 44 and 45, i.e., at the dash in the sequence amino acid NLG-MIM. This is typical of a membrane protein.

TABLE 7B
Encoded GPCR7 protein sequence (SEQ ID NO:22).

MSNTNGSAITEFILLGLTDCPELQSLLFVLFLVVYLVTLLGNLGMIMLMRLDSRLHTPMYFFLTNLAFVD
LCYTSNATPQMSTNIVSEKTISFAGCFTQCYIFIALLLTEFYMLAAMAYDRYVAIYDPLRYSVKTSRRVC
ICLATFPYVYGFSDGLFQAILTFRLTFCRSNVINHFYCADPPLIKLSCSDTYVKEHAMFISAGFNLSSSL
TIVLVSYAFILAAILRIKSAEGRHKAFSTCGSHMMAVTLFYGTLFCMYIRPPTDKTVEESKIIAVFYTFV
SPVLNPLIYSLRNKDVKQALKNVLR

In a search of sequence databases, it was found, for example, that the nucleic acid sequence of GPCR7 has 633 of 959 bases (66%) identical to a gb:GENBANK-ID:GGCOR2GEN lacc:X94742.1 mRNA from Gallus gallus COR2 (SEQ ID NO:51) (Table. 7C).

TABLE 7C
BLASTN of GPCR7 against COR2

>gb:GENBANK-ID:GGCOR2GEN|acc:X94742.1 G. gallus cor2 DNA for olfactory
receptor 2 - (SEQ ID NO:51) Gallus gallus, 996 bp.
Score = 1415 (212.3 bits), Expect = 5.9e-58, P = 5.9e-58
Identities = 633/959 (66%), Positives = 633/959 (66%),
Strand = Plus/Plus

| | |
|---|---|
| Query: | 7 AAGAGCAAACTGTCAGGA-AT-ATGTCCAACACAAATGGCAGTGCAATCACAGAATTCAT 64 |
| | \|\| \|\|\|\| \|\|\|\| \| \|\| \|\|\| \|\|\|\| \|\|\|\| \|\|\| \| \|\|\|\|\| \|\|\|\|\| \| |
| Sbjct: | 37 AACTGCAA-CTGTGTTGTGATGATGGCCAAGGGAAATCACAGCTCCATCACTGAATTTGT 95 |
| Query: | 65 TTTACTTGGGCT-CACAGATGCCCGGAACTCCAGTCTCTGCTTTTTGTGCTGTTTCTGG 123 |
| | \| \|\|\|\|\|\| \| \| \| \|\| \|\| \|\|\|\|\| \|\| \| \|\| \|\|\| \|\| \| \|\|\|\| \| |
| Sbjct: | 96 GCT-CTTGGGATTCTCTGAAAAGAGGGCCATCCAGGCTGTCTCTTTATGG-GCTTCTTG 153 |
| Query: | 124 TTGTT-TACCTCGTCACCCTGCTAGGCAACCTGGGCATGATAATGTTAATGAGACTGGAC 182 |
| | \|\| \| \|\|\|\|\| \|\|\|\| \|\|\|\|\|\|\|\|\|\|\| \|\|\|\|\|\|\|\|\|\| \| \|\| \|\| \|\| \|\|\|\|\|\| |
| Sbjct: | 154 CTGATCTACCTGATCACTCTGCTAGGCAATGTGGGCATGATGACATTGATCAGGCTGGAC 213 |
| Query: | 183 TCTCGCTTCACACGCCCATGTACTTCTTCCTCACTAACTTAGCCTTTGTGGATTTGTGC 242 |
| | \|\| \|\| \|\|\|\|\|\|\|\| \|\| \|\|\|\|\|\|\|\|\|\|\|\|\|\| \| \| \|\|\| \|\|\|\| \| \|\|\| \| \|\|\| |
| Sbjct: | 214 TCCCGGCTTCACACCCCTATGTACTTCTTCCTGAGCAGCTTGTCCTTCCTCGATATCTGC 273 |
| Query: | 243 TATACATC-A-AATGCAACCC-CGCA-GATGTC-GACTAATATC-GTATCTGAGAAGACC 296 |
| | \|\|\| \| \|\| \| \|\|\| \|\| \|\| \|\| \| \| \|\| \|\|\| \| \| \| \|\|\| \|\|\|\| \| |
| Sbjct: | 274 TATTCCTCCACAAT-CACTCCTCGAGTGCTCTCAGACC--TCCCAGCATCACAGAAAGTC 330 |
| Query: | 297 ATTTCCTTTGCTGGTTGCTTTACACAGTGCTACATTTTCATTGCCCTT-CTACTCACTGA 355 |
| | \|\|\|\|\|\| \|\|\| \|\|\| \| \|\|\|\|\|\| \|\| \| \| \| \|\| \| \|\| \| \|\| \|\|\| \|\| |
| Sbjct: | 331 ATTTCCCACTCTGCATGCCTGGCACAGTTTTATTTCTACGCTGTCTTTGCCAC-CACAGA 389 |
| Query: | 356 GTTTTACATGCTGGCAGCAATGGCCTATGACCGCTATGTGGCCATATATGACCCTCTGCG 415 |
| | \|\| \|\| \| \|\|\|\| \|\|\|\|\|\|\|\| \|\|\|\|\|\|\|\|\|\|\|\| \|\|\|\|\|\|\|\| \| \|\|\|\|\|\|\|\| |
| Sbjct: | 390 GTGCTATCTTTTGGCCGCAATGGCATATGACCGCTACGTGGCCATCTGCAGCCCTCTGCT 449 |
| Query: | 416 CTACAGTGTGAAA-ACGTCCAGGGAGAGTTTGCAT-CTGCTTG--GCCACATT-TCCCTAT 470 |
| | \|\|\| \|\| \| \| \|\|\|\|\|\| \|\|\|\|\|\|\|\| \| \|\|\|\|\| \| \|\| \| \| \|\|\| \| |
| Sbjct: | 450 CTAT-GTCTTCTCCATGTCCAGCAGAGTTTGTGTGCTGCTGGTTGCTGGCTCATACCT-T 507 |
| Query: | 471 GTCTATGGCTTCTCAGATGG-ACTCTTCCAGGCCATCCTGAC-CTTCCGCCTGACCTTCT 528 |
| | \|\|\| \|\| \|\| \| \| \|\|\| \|\| \|\|\| \| \| \|\| \| \|\| \| \|\| \|\| \|\|\|\|\|\| |
| Sbjct: | 508 GTCGG-GG-TTGTGA-ATGCCACCATTC-ACACAGGGCTTGCACTGCAGC-TGTCCTTCT 562 |
| Query: | 529 GTAGATCCAATGTCATCAACCACTTCTACTGTG-CTGACCCGCCGCTCATTAAGCTTTCT 587 |
| | \|\| \| \|\|\|\| \|\|\|\|\|\|\| \|\|\|\|\|\|\|\|\|\|\|\|\| \| \| \|\|\| \|\|\|\|\|\| \| \| \|\| \|\|\| |
| Sbjct: | 563 GTGGTCCCAACATCATCAATCACTTCTACTGTGACGGTCCC-CCGCTC-T-ACGCCATCT 619 |
| Query: | 588 TGTTCTGATACTT-ATGTCAAA-GAGCATGCCATGTTCATATCTGCT-GGCTTCAACCT- 643 |
| | \|\| \| \| \|\| \| \| \|\| \|\|\| \|\|\| \|\| \|\| \| \| \|\| \| \|\|\|\|\|\|\|\|\|\| \| |
| Sbjct: | 620 CGTGCACAGACCCCACCACCAACGAGATTGCGATATTTCT-TGTGGTTGGCTTCAACATG 678 |
| Query: | 644 CTCCAGCTCCCTCACCATCGTCAAGGTGTCCTATGCCTTCATTCTTGCT-GCCATCCTCC 702 |
| | \|\|\| \| \| \|\| \| \| \| \|\|\|\| \| \|\|\|\|\| \|\|\| \|\|\| \|\| \| \| \|\| \|\|\|\|\| |
| Sbjct: | 679 CTC-ATCACCAGCGTGACCATCTTCATCTCCTACACCTACATCCT-GTTCGCTGTCCTCA 736 |
| Query: | 703 GGAT-CAAATCAGCAG-AGGGAAGGCACAAGGCATTCTCCACCTGTGGTTCCCATATGAT 760 |
| | \|\|\|\| \|\| \| \|\|\|\| \| \|\|\| \|\| \| \|\|\| \| \|\|\|\|\|\|\|\| \|\|\|\| \|\|\|\|\| \|\| |
| Sbjct: | 737 GGATGCACA-CAGCTGCAGGCAAA-CGCAAAACCTTCTCCACGTGTGCGTCCCACCTGGC 794 |
| Query: | 761 GGCTGTCACCCTGTTTTATGGGACTCT-CTT-TTGCATGTATATAAGACC-ACCAACAGA 817 |
| | \| \|\|\|\|\|\|\|\| \|\| \|\|\|\| \|\|\|\| \|\| \|\| \|\|\|\|\|\| \| \| \|\| \| \| \|\|\| |
| Sbjct: | 795 CACCGTCACCCTATTCTATGC--CTCTGCTGGTTCCATGTACTCACGGCCCAGCTCCAGG 852 |
| Query: | 818 TAAGACT-GTTGAGGAATCTAAAATAATAGCTGTCTTTTACACCTTTGTGAGTCCGGTAC 876 |
| | \| \| \| \| \| \| \|\| \| \|\|\|\| \|\| \|\|\|\|\|\| \| \|\|\|\| \|\| \| \| |
| Sbjct: | 853 CACTCCCAGGACCTGGA-C-AAGGTGGCCTCTGTGTTCTACACCATGGTGACCCCCATGC 910 |
| Query: | 877 TTAATCCATTGATCTACAGTCTGAGGAATAAAGATGTGAAGCAGGCCTTGAAGAATGTCC 936 |
| | \| \|\| \|\| \| \|\|\|\|\|\|\|\| \|\|\|\|\|\|\|\| \| \|\| \|\| \|\|\| \| \| \|\| \|\|\| \|\| |
| Sbjct: | 911 TGAACCCCCTCATCTACAGCCTGAGGAACCAGGAGGTAAAGGATGTTTTAGGGAAAGTGA 970 |
| Query: | 937 TGAGATGAAATATTGTCA-TGACCA 960 |
| | \|\| \| \| \|\| \| \|\|\|\| \|\|\|\| \| |
| Sbjct: | 971 TGGGGAGGAAGAGTGTCTCTGACAA 995 |

The GPCR7 amino acid has 164 of 305 amino acid residues (53%) identical to, and 214 of 305 amino acid residues (70%) similar to, the 309 amino acid OR M72 (ptnr:TREMBLNEW-Acc No.:AAG09780) protein from Mus musculus OR M72, (SEQ ID NO:52) (Table 7D).

TABLE 7D
BLASTP alignments of GPCR7 against OR M72, (SEQ ID NO:52)

>ptnr:TREMBLNEW-ACC:AAG09780 ODORANT RECEPTOR M72 - Mus musculus(Mouse),
309 aa.
Score = 811 (285.5 bits), Expect = 1.2e-80, P = 1.2e-80
Identities = 164/305 (53%), Positives = 214/305 (70%)

| | |
|---|---|
| Query: | 1 MSNTNGSAITEFILLGLTDCPELQSLLFVLFLVVYLVTLLGNLGMIMLMRLDSRLHTPMY 60 |
| | \|+ \| \| +\|\|\|\|\| \|\|\|+ \|\|\|\| \| +\|\|\| +\|+\|\|++\|\|\|\|\|\| \|+ \|+\|+\|\|\|\|\|\| |
| Sbjct: | 1 MAAENQSTVTEFILRGLTNRPELQLPLLLLFLGIYIVTMVGNLGMITLIGLNSQLHTPMY 60 |
| Query: | 61 FFLTNLAFVDLCYTSNATPQMSTNIVSEKT-ISFAGCFTQCYIFIALLLTEFYMLAAMAY 119 |
| | \|\|\|+\|\|+ \|\|\|\|\|+\|++\|\|+\|++\|+\|\|++ \|\|+ \|\| +\| \| \|+ ++ \| \|\|\| \|\|\| |
| Sbjct: | 61 FFLSNLSLVDLCYSSCITPKMLINFVSQRNLISYVGCMSQLYFFLVFVIAECYMLTVMAY 120 |
| Query: | 120 DRYVAIYDPLRYSVKTSRRVCICLATFPYVYGFSDGLFQAILTFRLTFCRSNVINHFYCA 179 |
| | \|\|\|\|\|\| \|\| \|++ \| +\| \| \| \| \| + \| +\| +\| ++\|+\|++\| |
| Sbjct: | 121 DRYVAICQPLLYNIIMSPALCSLLVVFVYAMGLIGSTIETSLMLKLNYCE-DLISHYFCD 179 |
| Query: | 180 DPPLIKLSCSDTYVKEHAMFISAGFNLS-SSLTIVLVSYAFILAAILRIKSAEGRHKAFS 238 |
| | \|+\|\|\|\| \|\| \| \|+\| \|\|\|\|+ +\|\|\| \|\|+\|\|\|\|\|\|++\|\|\|\| \| \|\|\| \|\|\|\| |
| Sbjct: | 180 ILPLMKLSCSSTYDIEMAVFFLAGFNIIVTSLT-VLISYAFILSSILRISSNEGRSKAFS 238 |
| Query: | 239 TCGSHMMAVTLFYGTLFCMYIRPPTDKTVEESKIIAVFYTFVSPVLPNLIYSLRNKDVKQ 298 |
| | \|\| \|\| \|\| \|\|\|\|+ \|\|++\| \| ++ + + +\|\|\|\| \| \|+ \|\|\|\|\|\|\|\|\|\|+\|\| |
| Sbjct: | 239 TCSSHFAAVGLFYGSTAFMYLKPSTASSLAQENVASVFYTTIVIPMFNPLIYSLRNKEVKT 298 |
| Query: | 299 ALKNVLR 305 |
| | \|\| \|\| |
| Sbjct: | 299 ALDKTLR 305 |

The presence of identifiable domains in GPCR7 was determined by searches using algorithms such as PROSITE, Blocks, Pfam, ProDomain, and Prints followed by determining the Interpro number by crossing the domain match (or numbers) using the Interpro website (http:www.ebi.ac.uk/interpro/). The results indicate that this protein contains the following protein domains (as defined by Interpro) at the indicated positions: domain name 7tm_{—}1 (InterPro) 7 transmembrane receptor (rhodopsin family) at amino acid positions 41 to 289. This indicates that the sequence of GPCR7 has properties similar to those of other proteins known to contain this domain.

GPCR7 maps to chromosome 11. This information was assigned using the Online Mendelian Inheritance in Man (OMIM) database, the electronic northern bioinformatic tool implemented by CuraGen Corporation, public ESTs, public literature references and/or genomic clone homologies. This was executed to derive the chromosomal mapping of the Genomic clones, literature references and/or EST sequences that were included in the invention.

GPCR7 is expressed in at least the following tissues: Apical microvilli of the retinal pigment epithelium, arterial (aortic), basal forebrain, brain, Burkitt lymphoma cell lines, corpus callosum, cardiac (atria and ventricle), caudate nucleus, CNS and peripheral tissue, cerebellum, cerebral cortex, colon, cortical neurogenic cells, endothelial (coronary artery and umbilical vein) cells, palate epithelia, eye, neonatal eye, frontal cortex, fetal hematopoietic cells, heart, hippocampus, hypothalamus, leukocytes, liver, fetal liver, lung, lung lymphoma cell lines, fetal lymphoid tissue, adult lymphoid tissue, Those that express MHC II and III nervous, medulla, subthalamic nucleus, ovary, pancreas, pituitary, placenta, pons, prostate, putamen, serum, skeletal muscle, small intestine, smooth muscle (coronary artery in aortic) spinal cord, spleen, stomach, taste receptor cells of the tongue, testis, thalamus, and thymus tissue. This information was derived by determining the tissue sources of the sequences that were included in the invention including but not limited to Public EST sources, Genomic Clone sources, Literature sources, and/or RACE sources.

The protein similarity information, expression pattern, and map location for the GPCR7 protein and nucleic acid suggest that GPCR7 may have important structural and/or physiological functions characteristic of the Olfactory Receptor family. Therefore, GPCR7 are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

GPCR7 is useful in potential diagnostic and therapeutic applications implicated in various GPCR- or OR-related diseases and disorders described below and/or other pathologies. For example, the compositions of GPCR7 will have efficacy for treatment of patients suffering from:: Familial Mediterranian Fever, developmental diseases, MHCII and III diseases (immune diseases), Taste and scent detectability Disorders, Burkitt's lymphoma, Corticoneurogenic disease, Signal Transduction pathway disorders, Retinal diseases including those involving photoreception, Cell Growth rate disorders; Cell Shape disorders, Feeding disorders;control of feeding; potential obesity due to over-eating; potential disorders due to starvation (lack of apetite), noninsulin-dependent diabetes mellitus (NIDDM1), bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy(DRPLA) Hypophosphatemic rickets, autosomal dominant (2) Acrocallosal syndrome and dyskinesias, such as Huntington's disease or Gilles de la Tourette syndrome and/or other pathologies and disorders of the like. The polypeptides can be used as immunogens to produce antibodies specific for GPCR7, and as vaccines. They can also be used to screen for potential agonist and antagonist compounds. For example, a cDNA encoding the GPCR7-like protein may be useful in gene therapy, and the GPCR7-like protein may be useful when administered to a subject in need thereof. By way of nonlimiting example, the compositions of GPCR7 will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders. The GPCR7 nucleic acid and protein, or fragments thereof, may further be useful in diagnostic applications, wherein the presence or amount of the nucleic acid or the protein are to be assessed. These materials are further useful in the generation of antibodies that bind immunospecifically to GPCR7 for use in therapeutic or diagnostic methods. Other GPCR-related diseases and disorders are contemplated.

These materials are further useful in the generation of antibodies that bind immunospecifically to the novel GPCR7 substances for use in therapeutic or diagnostic methods. These antibodies may be generated according to methods known in the art, using prediction from hydrophobicity charts, as described in the “Anti-GPCRX Antibodies” section below. In one embodiment, a contemplated GPCR7 epitope is from aa 15 to 70. In another embodiment, a GPCR7 epitope is from aa 85 to 125. In additional embodiments, GPCR7 epitopes are from aa 140 to 175, from aa 210 to 235, from aa 240 to 260, and from aa 275 to 290.

A summary of the GPCRX nucleic acids and proteins of the invention is provided in Table 8A. A summary of homologous sequences identified in searches of available sequence databases is provided in Table 8B.

TABLE 8A
Summary Of Nucleic Acids And Proteins Of The Invention

| | | | Nucleic Acid | Amino Acid |
|---|---|---|---|---|
| Name | Tables | Clone; Description of Homolog | SEQ ID NO | SEQ ID NO |
| GPCR1 | 1A, 1B, | AL031943_A; CG54236-02; GPCR-like | 1 | 2 |
| | 1C | protein, cysteinyl leukotriene receptor-like | | |
| | | protein | | |
| GPCR2 | 2A, 2B | AC022289_A; OR-like protein | 3 | 4 |
| | 2C, 2D | AC022289_A1; OR-like protein | 5 | 6 |
| GPCR3 | 3A, 3B | AP001112_A; OR-like protein | 7 | 8 |
| GPCR4 | 4A, 4B | AP001112_B; OR-like protein | 9 | 10 |
| | 4G, 4D | AC020597A; OR-like protein | 11 | 12 |
| GPCR5 | 5A, 5B | AP001112_C; OR-like protein | 13 | 14 |
| | 5C, 5D | AC0170103B1; OR-like protein | 15 | 16 |
| | 5E, 5F | CG50173-01; OR-like protein | 17 | 18 |
| GPCR6 | 6A, 6B | AP001112_D; OR-like protein | 19 | 20 |
| GPCR7 | 7A, 7B | AP001112_da1; OR-like protein | 21 | 22 |
| GPCR1 | Example 3 | Ag269S Forward | 63 | |
| GPCR1 | Example 3 | Ag269S Probe | 64 | |
| GPCR1 | Example 3 | Ag2695 Reverse | 65 | |

TABLE 8B
Summary of Query Sequences Disclosed

| Table | Database | Acc. No. | Sequence Name | Species | SEQ ID NO. |
|---|---|---|---|---|---|
| 1D | GenBank | L06109 | activated T cell-specific G PCR mRNA | chicken | 23 |
| 1E | GenBank | XM_007164.1 | cysteinyl leukotriene CysLT2 receptor | human | 24 |
| 1F | trEmblnew | CAA73144 | P2Y-Like G-Protein Coupled Receptor | human | 25 |
| 1G, 1H | GenBank | XP_007164 | translation, cysteinyl leukotriene CysLT2 | human | 26 |
| | | | receptor | | |
| 1H | Patp | W75799 | unknown | human | 27 |
| 1H | strpEmb1 | P34996 | P2Y Purinoceptor 1 | chicken | 28 |
| 1I, 2J, 3H, | Pfam | 7tm_1 | 7 transmembrane receptor (rhodopsin | consensus | 29 |
| 4O, 5M, 6K | | | family) fragment ; residues 1-180 | | |
| 2E | GenBank | 450948 | TB 567 | rat | 30 |
| 2F | GenBank | AFD65860 | ORD 3; residues 437-644 | human | 31 |
| 2F | GenBank | AFD65860 | ORD-3; residues 121-219 | human | 32 |
| 2G, 4N, 5L, | SwissProt | Q13606 | OLF-1 | human | 33 |
| 6J | | | | | |
| 2H, 4M | strpEmb1 | CAA64370 | OR | chicken | 34 |
| 2I | SWISS | P37070 | OLF | chicken | 35 |
| 2I | GenBank | Q63395 | OLF | rat | 36 |
| 2J, 4O, 5M, | Pfam | 7tm-1 | 7 transmembrane receptor (rhodopsin | consensus | 37 |
| 6K | | | family) fragment; residues 310-377 | | |
| 3C | GenBank | AF045577 | OR93 | chimp | 38 |
| 3D | GenBank | NM_013728 | ORfr 4-3; residues 835-907 | mouse | 39 |
| 3D | GenBank | NM_013728 | ORfr 4-3; residues 163-210 | mouse | 40 |
| 3E, 3G, 4I | sptrEmbl | O77756 | OLF; residues 5-309 | chimp | 41 |
| 4N | | | | | |
| 3F, 3G, 4N | GenBank | AF20365 | OLF | mouse | 42 |
| 3G, 4J, 4N, | sptrEmbl | O77758 | OLF | gibbon | 43 |
| 5H, 5L, 6J | | | | | |
| 4E | GenBank | U50948 | TB 567 | rat | 44 |
| 4F | GenBank | AF247656 | M72; residues 821-890 | mouse | 45 |
| 4F | GenBank | AF247656 | M72; residues 160-201 | mouse | 46 |
| 4G | GenBank | AF282291 | ORK42 | mouse | 47 |
| 4H | GenBank | AF282298 | OR K40 | mouse | 48 |
| 4K | GenBank | AF282291 | OR K42 | mouse | 49 |
| 4L | GenBank | NP_006628 | OR 5I1 | human | 50 |
| 5G, 6C | GenBank | X94742 | COR2 | chicken | 51 |
| 5I | GenBank | AAG09870 | M72 | mouse | 52 |
| 5J | GenBank | AAG39876 | OR K42 | mouse | 53 |
| 5K | GenBank | AAG29379 | M71 | mouse | 54 |
| 5L | sptrEmb1 | Q63394 | OLF | rat | 55 |
| 6D, 6J | sptrEmb1 | Q90808 | OR4 | chicken | 56 |
| 6E | GenBank | AAC63969 | OR93Ch | chimp | 57 |
| 6F | GenBank | AAC63971 | OR93Gib | gibbon | 58 |
| 6G | EMBL | CAA64368 | COR2 | chicken | 59 |
| 6H | GenBank | AAG39871 | K30 | mouse | 60 |
| 6I | GenBank | AAG39856 | OR K11 | mouse | 61 |
| 6J | GenBank | NP_068632 | OR G264617 | rat | 62 |

GPCRX Nucleic Acids and Polypeptides

One aspect of the invention pertains to isolated nucleic acid molecules that encode GPCRX polypeptides or biologically-active portions thereof. Also included in the invention are nucleic acid fragments sufficient for use as hybridization probes to identify GPCRX-encoding nucleic acids (e.g., GPCRX mRNAs) and fragments for use as PCR primers for the amplification and/or mutation of GPCRX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An GPCRX nucleic acid can encode a mature GPCRX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated GPCRX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 11, 13, 15, 17, 19 and 21, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19 and 21 as a hybridization probe, GPCRX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GPCRX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS: 1, 3, 5, 7, 9, 11, 13, 15, 17, 19 and 21, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19 and 21, or a portion of this nucleotide sequence (e.g., a fragment that can be used as a probe or primer or a fragment encoding a biologically-active portion of an GPCRX polypeptide). A nucleic acid molecule that is complementary to the nucleotide sequence shown in SEQ ID NOS:1, 3, 5, 7, 9, 11, 13, 15, 17, 19 and 21, is one that is sufficiently complementary to the nucleotide sequence shown in SEQ ID NOS: 1, 3, 5, 7, 9, 11, 13, 15, 17, 19 and 21, that it can hydrogen bond with little or no mismatches to the nucleotide sequence shown in SEQ ID NOS:1, 3, 5, 7, 9, 11, 13, 15, 17, 19 and 21, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of GPCRX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an GPCRX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human GPCRX protein. Homologous nucleic acid sequences include those nucleic acid sequences that encode conservative amino acid substitutions (see below) in SEQ ID NOS:2, 4, 6, 8, 10, 12, 14, 16, 18, 20 and 22, as well as a polypeptide possessing GPCRX biological activity. Various biological activities of the GPCRX proteins are described below.

An GPCRX polypeptide is encoded by the open reading frame (“ORF”) of an GPCRX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human GPCRX genes allows for the generation of probes and primers designed for use in identifying and/or cloning GPCRX homologues in other cell types, e.g. from other tissues, as well as GPCRX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS: 1, 3, 5, 7, 9, 11, 13, 15, 17, 19 and 21; or an anti-sense strand nucleotide sequence of SEQ ID NOS:1, 3, 5, 7, 9, 11, 13, 15, 17, 19 and 21; or of a naturally occurring mutant of SEQ ID NOS: 1, 3, 5, 7, 9, 11, 13, 15, 17, 19 and 21.

Probes based on the human GPCRX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an GPCRX protein, such as by measuring a level of an GPCRX-encoding nucleic acid in a sample of cells from a subject e.g., detecting GPCRX mRNA levels or determining whether a genomic GPCRX gene has been mutated or deleted.

“A polypeptide having a biologically-active portion of an GPCRX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of GPCRX” can be prepared by isolating a portion of SEQ ID NOS:1, 3, 5, 7, 9, 11, 13, 15, 17, 19 and 21, that encodes a polypeptide having an GPCRX biological activity (the biological activities of the GPCRX proteins are described below), expressing the encoded portion of GPCRX protein (e.g., by recombinant expression in vitro) and assessing the activity of the encoded portion of GPCRX.

GPCRX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19 and 21, due to degeneracy of the genetic code and thus encode the same GPCRX proteins as that encoded by the nucleotide sequences shown in SEQ ID NO NOS:1, 3, 5, 7, 9, 11, 13, 15, 17, 19 and 21. In another embodiment, an isolated nucleic acid molecule of the invention has a nucleotide sequence encoding a protein having an amino acid sequence shown in SEQ ID NO S:2, 4, 6, 8, 10, 12, 14, 16, 18, 20 and 22.

In addition to the human GPCRX nucleotide sequences shown in SEQ ID NOS: 1, 3, 5, 7, 9, 11, 13, 15, 17, 19 and 21, it will be appreciated by those skilled in the art that DNA sequence polymorphisms that lead to changes in the amino acid sequences of the GPCRX polypeptides may exist within a population (e.g., the human population). Such genetic polymorphism in the GPCRX genes may exist among individuals within a population due to natural allelic variation. As used herein, the terms “gene” and “recombinant gene” refer to nucleic acid molecules comprising an open reading frame (ORF) encoding an GPCRX protein, preferably a vertebrate GPCRX protein. Such natural allelic variations can typically result in 1-5% variance in the nucleotide sequence of the GPCRX genes. Any and all such nucleotide variations and resulting amino acid polymorphisms in the GPCRX polypeptides, which are the result of natural allelic variation and that do not alter the functional activity of the GPCRX polypeptides, are intended to be within the scope of the invention.

Moreover, nucleic acid molecules encoding GPCRX proteins from other species, and thus that have a nucleotide sequence that differs from the human sequence of SEQ ID NOS: 1, 3, 5, 7, 9, 11, 13, 15, 17, 19 and 21, are intended to be within the scope of the invention. Nucleic acid molecules corresponding to natural allelic variants and homologues of the GPCRX cDNAs of the invention can be isolated based on their homology to the human GPCRX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19 and 21. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding GPCRX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6X SSC, 50 mM Tris-HCI (pH 7.5), 1 mM EDTA, 0.02% PVP, 0.02% Ficoll, 0.02% BSA, and 500 mg/ml denatured salmon sperm DNA at 65° C., followed by one or more washes in 0.2X SSC, 0.01% BSA at 50° C. An isolated nucleic acid molecule of the invention that hybridizes under stringent conditions to the sequences of SEQ ID NOS:1, 3, 5, 7, 9, 11, 13, 15, 17, 19 and 21, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19 and 21, or fragments, analogs or derivatives thereof, under conditions of moderate stringency is provided. A non-limiting example of moderate stringency hybridization conditions are hybridization in 6X SSC, 5X Denhardt's solution, 0.5% SDS and 100 mg/ml denatured salmon sperm DNA at 55° C., followed by one or more washes in 1X SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences of SEQ ID NOS: 1, 3, 5, 7, 9, 11, 13, 15, 17, 19 and 21, or fragments, analogs or derivatives thereof, under conditions of low stringency, is provided.

A non-limiting example of low stringency hybridization conditions are hybridization in 35% formamide, 5X SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2X SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of GPCRX sequences that may exist in the population, the skilled artisan will further appreciate that changes can be introduced by mutation into the nucleotide sequences of SEQ ID NO NOS:1, 3, 5, 7, 9, 11, 13, 15, 17, 19 and 21, thereby leading to changes in the amino acid sequences of the encoded GPCRX proteins, without altering the functional ability of said GPCRX proteins. For example, nucleotide substitutions leading to amino acid substitutions at “non-essential” amino acid residues can be made in the sequence of SEQ ID NOS:2, 4, 6, 8, 10, 12, 14, 16, 18, 20 and 22. A “non-essential” amino acid residue is a residue that can be altered from the wild-type sequences of the GPCRX proteins without altering their biological activity, whereas an “essential” amino acid residue is required for such biological activity. For example, amino acid residues that are conserved among the GPCRX proteins of the invention are predicted to be particularly non-amenable to alteration. Amino acids for which conservative substitutions can be made are well-known within the art.

Another aspect of the invention pertains to nucleic acid molecules encoding GPCRX proteins that contain changes in amino acid residues that are not essential for activity. Such GPCRX proteins differ in amino acid sequence from SEQ ID NOS:2, 4, 6, 8, 10, 12, 14, 16, 18, 20 and 22, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8, 10, 12, 14, 16, 18, 20 and 22. Preferably, the protein encoded by the nucleic acid molecule is at least about 60% homologous to SEQ ID NOS:2, 4, 6, 8, 10, 12, 14, 16, 18, 20 and 22; more preferably at least about 70% homologous to SEQ ID NOS:2, 4, 6, 8, 10, 12, 14, 16, 18, 20 and 22; still more preferably at least about 80% homologous to SEQ ID NOS:2, 4, 6, 8, 10, 12, 14, 16, 18, 20 and 22; even more preferably at least about 90% homologous to SEQ ID NOS:2, 4, 6, 8, 10, 12, 14, 16, 18, 20 and 22; and most preferably at least about 95% homologous to SEQ ID NOS:2, 4, 6, 8, 10, 12, 14, 16, 18, 20 and 22.

An isolated nucleic acid molecule encoding an GPCRX protein homologous to the protein of SEQ ID NOS:2, 4, 6, 8, 10, 12, 14, 16, 18, 20 and 22, can be created by introducing one or more nucleotide substitutions, additions or deletions into the nucleotide sequence of SEQ ID NOS:1, 3, 5, 7, 9, 11, 13, 15, 17, 19 and 21, such that one or more amino acid substitutions, additions or deletions are introduced into the encoded protein.

Mutations can be introduced into SEQ ID NOS:2, 4, 6, 8, 10, 12, 14, 16, 18, 20 and 22, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the GPCRX protein is replaced with another amino acid residue from the same side chain family. Alternatively, in another embodiment, mutations can be introduced randomly along all or part of an GPCRX coding sequence, such as by saturation mutagenesis, and the resultant mutants can be screened for GPCRX biological activity to identify mutants that retain activity. Following mutagenesis of SEQ ID NOS:2, 4, 6, 8, 10, 12, 14, 16, 18, 20 and 22, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant GPCRX protein can be assayed for (i) the ability to form protein:protein interactions with other GPCRX proteins, other cell-surface proteins, or biologically-active portions thereof, (ii) complex formation between a mutant GPCRX protein and an GPCRX ligand; or (iii) the ability of a mutant GPCRX protein to bind to an intracellular target protein or biologically-active portion thereof, (e.g. avidin proteins).

In yet another embodiment, a mutant GPCRX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19 and 21, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire GPCRX coding strand, or to only a portion thereof. Nucleic acid molecules encoding fragments, homologs, derivatives and analogs of an GPCRX protein of SEQ ID NOS:2, 4, 6, 8, 10, 12, 14, 16, 18, 20 and 22; or antisense nucleic acids complementary to an GPCRX nucleic acid sequence of SEQ ID NOS: 1, 3, 5, 7, 9, 11, 13, 15, 17, 19 and 21, are additionally provided.

In one embodiment, an antisense nucleic acid molecule is antisense to a “coding region” of the coding strand of a nucleotide sequence encoding an GPCRX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the GPCRX protein. The term “noncoding region” refers to 5′ and 3′ sequences which flank the coding region that are not translated into amino acids (i.e., also referred to as 5′ and 3′ untranslated regions).

Given the coding strand sequences encoding the GPCRX protein disclosed herein, antisense nucleic acids of the invention can be designed according to the rules of Watson and Crick or Hoogsteen base pairing. The antisense nucleic acid molecule can be complementary to the entire coding region of GPCRX mRNA, but more preferably is an oligonucleotide that is antisense to only a portion of the coding or noncoding region of GPCRX mRNA. For example, the antisense oligonucleotide can be complementary to the region surrounding the translation start site of GPCRX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an GPCRX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense imolecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong po II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual p-units, the strands run parallel to each other. See, e.g., Gaultier, et al., 1987. Nucl. Acids Res. 15:

6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave GPCRX mRNA transcripts to thereby inhibit translation of GPCRX mRNA. A ribozyme having specificity for an GPCRX-encoding nucleic acid can be designed based upon the nucleotide sequence of an GPCRX cDNA disclosed herein (i.e., SEQ ID NOS: 1, 3, 5, 7, 9, 11, 13, 15, 17, 19 and 21). For example, a derivative of a Tetrahymena L-19 IVS RNA can be constructed in which the nucleotide sequence of the active site is complementary to the nucleotide sequence to be cleaved in an GPCRX-encoding mRNA. See, e.g., U.S. Pat. No. 4,987,071 to Cech, et al. and U.S. Pat. No. 5,116,742 to Cech, et al. GPCRX mRNA can also be used to select a catalytic RNA having a specific ribonuclease activity from a pool of RNA molecules. See, e.g., Bartel et al., (1993) Science 261:1411-1418.

Alternatively, GPCRX gene expression can be inhibited by targeting nucleotide sequences complementary to the regulatory region of the GPCRX nucleic acid (e.g., the GPCRX promoter and/or enhancers) to form triple helical structures that prevent transcription of the GPCRX gene in target cells. See, e.g., Helene, 1991. Anticancer Drug Des. 6: 569-84; Helene, et al. 1992. Ann. NAY. Acad. Sci. 660: 27-36; Maher, 1992. Bioassays 14: 807-15.

In various embodiments, the GPCRX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of GPCRX can be used in therapeutic and diagnostic applications. For example, PNAs can be used as antisense or antigene agents for sequence-specific modulation of gene expression by, e.g., inducing transcription or translation arrest or inhibiting replication. PNAs of GPCRX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996, supra); or as probes or primers for DNA sequence and hybridization (see, Hyrup, et al., 1996, supra; Perry-O'Keefe, et al., 1996. supra).

In another embodiment, PNAs of GPCRX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of GPCRX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, etal., 1996. supra).

The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

GPCRX Polypeptides

A polypeptide according to the invention includes a polypeptide including the amino acid sequence of GPCRX polypeptides whose sequences are provided in SEQ ID NOS:2, 4, 6, 8, 10, 12, 14, 16, 18, 20 and 22. The invention also includes a mutant or variant protein any of whose residues may be changed from the corresponding residues shown in SEQ ID NOS:2, 4, 6, 8, 10, 12, 14, 16, 18, 20 and 22, while still encoding a protein that maintains its GPCRX activities and physiological functions, or a functional fragment thereof.

In general, an GPCRX variant that preserves GPCRX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated GPCRX proteins, and biologically-active portions thereof, or derivatives, fragments, analogs or homologs thereof. Also provided are polypeptide fragments suitable for use as immunogens to raise anti-GPCRX antibodies. In one embodiment, native GPCRX proteins can be isolated from cells or tissue sources by an appropriate purification scheme using standard protein purification techniques. In another embodiment, GPCRX proteins are produced by recombinant DNA techniques. Alternative to recombinant expression, an GPCRX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the GPCRX protein is derived, or substantially free from chemical precursors or other chemicals when chemically synthesized. The language “substantially free of cellular material” includes preparations of GPCRX proteins in which the protein is separated from cellular components of the cells from which it is isolated or recombinantly-produced. In one embodiment, the language “substantially free of cellular material” includes preparations of GPCRX proteins having less than about 30% (by dry weight) of non-GPCRX proteins (also referred to herein as a “contaminating protein”), more preferably less than about 20% of non-GPCRX proteins, still more preferably less than about 10% of non-GPCRX proteins, and most preferably less than about 5% of non-GPCRX proteins. When the GPCRX protein or biologically-active portion thereof is recombinantly-produced, it is also preferably substantially free of culture medium, i.e., culture medium represents less than about 20%, more preferably less than about 10%, and most preferably less than about 5% of the volume of the GPCRX protein preparation.

The language “substantially free of chemical precursors or other chemicals” includes preparations of GPCRX proteins in which the protein is separated from chemical precursors or other chemicals that are involved in the synthesis of the protein. In one embodiment, the language “substantially free of chemical precursors or other chemicals” includes preparations of GPCRX proteins having less than about 30% (by dry weight) of chemical precursors or non-GPCRX chemicals, more preferably less than about 20% chemical precursors or non-GPCRX chemicals, still more preferably less than about 10% chemical precursors or non-GPCRX chemicals, and most preferably less than about 5% chemical precursors or non-GPCRX chemicals.

Biologically-active portions of GPCRX proteins include peptides comprising amino acid sequences sufficiently homologous to or derived from the amino acid sequences of the GPCRX proteins (e.g., the amino acid sequence shown in SEQ ID NOS:2, 4, 6, 8, 10, 12, 14, 16, 18, 20 and 22) that include fewer amino acids than the full-length GPCRX proteins, and exhibit at least one activity of an GPCRX protein. Typically, biologically-active portions comprise a domain or motif with at least one activity of the GPCRX protein. A biologically-active portion of an GPCRX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native GPCRX protein.

In an embodiment, the GPCRX protein has an amino acid sequence shown in SEQ ID NOS:2, 4, 6, 8, 10, 12, 14, 16, 18, 20 and 22. In other embodiments, the GPCRX protein is substantially homologous to SEQ ID NOS:2, 4, 6, 8, 10, 12, 14, 16, 18, 20 and 22, and retains the functional activity of the protein of SEQ ID NOS:2, 4, 6, 8, 10, 12, 14, 16, 18, 20 and 22, yet differs in amino acid sequence due to natural allelic variation or mutagenesis, as described in detail, below. Accordingly, in another embodiment, the GPCRX protein is a protein that comprises an amino acid sequence at least about 45% homologous to the amino acid sequence of SEQ ID NOS:2, 4, 6, 8, 10, 12, 14, 16, 18, 20 and 22, and retains the functional activity of the GPCRX proteins of SEQ ID NOS:2, 4, 6, 8, 10, 12, 14, 16, 18, 20 and 22.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19 and 21.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides GPCRX chimeric or fusion proteins. As used herein, an GPCRX “chimeric protein” or “fusion protein” comprises an GPCRX polypeptide operatively-linked to a non-GPCRX polypeptide. An “GPCRX polypeptide” refers to a polypeptide having an amino acid sequence corresponding to an GPCRX protein (SEQ ID NOS:2, 4, 6, 8, 10, 12, 14, 16, 18, 20 and 22), whereas a “non-GPCRX polypeptide” refers to a polypeptide having an amino acid sequence corresponding to a protein that is not substantially homologous to the GPCRX protein, e.g., a protein that is different from the GPCRX protein and that is derived from the same or a different organism. Within an GPCRX fusion protein the GPCRX polypeptide can correspond to all or a portion of an GPCRX protein. In one embodiment, an GPCRX fusion protein comprises at least one biologically-active portion of an GPCRX protein. In another embodiment, an GPCRX fusion protein comprises at least two biologically-active portions of an GPCRX protein. In yet another embodiment, an GPCRX fusion protein comprises at least three biologically-active portions of an GPCRX protein. Within the fusion protein, the term “operatively-linked” is intended to indicate that the GPCRX polypeptide and the non-GPCRX polypeptide are fused in-frame with one another. The non-GPCRX polypeptide can be fused to the N-terminus or C-terminus of the GPCRX polypeptide.

In one embodiment, the fusion protein is a GST-GPCRX fusion protein in which the GPCRX sequences are fused to the C-terminus of the GST (glutathione S-transferase) sequences. Such fusion proteins can facilitate the purification of recombinant GPCRX polypeptides.

In another embodiment, the fusion protein is an GPCRX protein containing a heterologous signal sequence at its N-terminus. In certain host cells (e.g., mammalian host cells), expression and/or secretion of GPCRX can be increased through use of a heterologous signal sequence.

In yet another embodiment, the fusion protein is an GPCRX-immunoglobulin fusion protein in which the GPCRX sequences are fused to sequences derived from a member of the immunoglobulin protein family. The GPCRX-immunoglobulin fusion proteins of the invention can be incorporated into pharmaceutical compositions and administered to a subject to inhibit an interaction between an GPCRX ligand and an GPCRX protein on the surface of a cell, to thereby suppress GPCRX-mediated signal transduction in vivo. The GPCRX-immunoglobulin fusion proteins can be used to affect the bioavailability of an GPCRX cognate ligand. Inhibition of the GPCRX ligand/GPCRX interaction may be useful therapeutically for both the treatment of proliferative and differentiative disorders, as well as modulating (e.g. promoting or inhibiting) cell survival. Moreover, the GPCRX-immunoglobulin fusion proteins of the invention can be used as immunogens to produce anti-GPCRX antibodies in a subject, to purify GPCRX ligands, and in screening assays to identify molecules that inhibit the interaction of GPCRX with an GPCRX ligand.

An GPCRX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An GPCRX-encoding nucleic acid can be cloned into such an expression vector such that the fusion moiety is linked in-frame to the GPCRX protein.

GPCRX Agonists and Antagonists

The invention also pertains to variants of the GPCRX proteins that function as either GPCRX agonists (i.e., mimetics) or as GPCRX antagonists. Variants of the GPCRX protein can be generated by mutagenesis (e.g., discrete point mutation or truncation of the GPCRX protein). An agonist of the GPCRX protein can retain substantially the same, or a subset of, the biological activities of the naturally occurring form of the GPCRX protein. An antagonist of the GPCRX protein can inhibit one or more of the activities of the naturally occurring form of the GPCRX protein by, for example, competitively binding to a downstream or upstream member of a cellular signaling cascade which includes the GPCRX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the GPCRX proteins.

Variants of the GPCRX proteins that function as either GPCRX agonists (i.e., mimetics) or as GPCRX antagonists can be identified by screening combinatorial libraries of mutants (e.g., truncation mutants) of the GPCRX proteins for GPCRX protein agonist or antagonist activity. In one embodiment, a variegated library of GPCRX variants is generated by combinatorial mutagenesis at the nucleic acid level and is encoded by a variegated gene library. A variegated library of GPCRX variants can be produced by, for example, enzymatically ligating a mixture of synthetic oligonucleotides into gene sequences such that a degenerate set of potential GPCRX sequences is expressible as individual polypeptides, or alternatively, as a set of larger fusion proteins (e.g., for phage display) containing the set of GPCRX sequences therein. There are a variety of methods which can be used to produce libraries of potential GPCRX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCRX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the GPCRX protein coding sequences can be used to generate a variegated population of GPCRX fragments for screening and subsequent selection of variants of an GPCRX protein. In one embodiment, a library of coding sequence fragments can be generated by treating a double stranded PCR fragment of an GPCRX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the GPCRX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GPCRX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify GPCRX variants. See, e.g., Arkin and Yourvan, 1992. Proc. Natl. Acad. Sci. USA 89: 7811-7815; Delgrave, et al., 1993. Protein Engineering 6:327-331.

Anti-GPCRX Antibodies

The invention encompasses antibodies and antibody fragments, such as F_{ab} or (F_{ab)2}, that bind immunospecifically to any of the GPCRX polypeptides of said invention.

An isolated GPCRX protein, or a portion or fragment thereof, can be used as an immunogen to generate antibodies that bind to GPCRX polypeptides using standard techniques for polyclonal and monoclonal antibody preparation. The full-length GPCRX proteins can be used or, alternatively, the invention provides antigenic peptide fragments of GPCRX proteins for use as immunogens. The antigenic GPCRX peptides comprises at least 4 amino acid residues of the amino acid sequence shown in SEQ ID NO NOS:2, 4, 6, 8, 10, 12, 14, 16, 18, 20 and 22, and encompasses an epitope of GPCRX such that an antibody raised against the peptide forms a specific immune complex with GPCRX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of GPCRX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol Biol. 157: 105-142, each incorporated herein by reference in their entirety).

As disclosed herein, GPCRX protein sequences of SEQ ID NOS:2, 4, 6, 8, 10, 12, 14, 16, 18, 20 and 22, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as GPCRX. Such antibodies include, but are not limited to, polyclonal, monoclonal, chimeric, single chain, Fab and F(_{ab)2} fragments, and an F_{ab} expression library. In a specific embodiment, antibodies to human GPCRX proteins are disclosed. Various procedures known within the art may be used for the production of polyclonal or monoclonal antibodies to an GPCRX protein sequence of SEQ ID NOS:2, 4, 6, 8, 10, 12, 14, 16, 18, 20 and 22, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed GPCRX protein or a chemically-synthesized GPCRX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against GPCRX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of GPCRX. A monoclonal antibody composition thus typically displays a single binding affinity for a particular GPCRX protein with which it immunoreacts. For preparation of monoclonal antibodies directed towards a particular GPCRX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc NatlAcad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an GPCRX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an GPCRX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an GPCRX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-GPCRX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an GPCRX protein is facilitated by generation of hybridomas that bind to the fragment of an GPCRX protein possessing such a domain. Thus, antibodies that are specific for a desired domain within an GPCRX protein, or derivatives, fragments, analogs or homologs thereof, are also provided herein.

Anti-GPCRX antibodies may be used in methods known within the art relating to the localization and/or quantitation of an GPCRX protein (e.g., for use in measuring levels of the GPCRX protein within appropriate physiological samples, for use in diagnostic methods, for use in imaging the protein, and the like). In a given embodiment, antibodies for GPCRX proteins, or derivatives, fragments, analogs or homologs thereof, that contain the antibody derived binding domain, are utilized as pharmacologically-active compounds (hereinafter “Therapeutics”).

An anti-GPCRX antibody (e.g., monoclonal antibody) can be used to isolate an GPCRX polypeptide by standard techniques, such as affinity chromatography or immunoprecipitation. An anti-GPCRX antibody can facilitate the purification of natural GPCRX polypeptide from cells and of recombinantly-produced GPCRX polypeptide expressed in host cells. Moreover, an anti-GPCRX antibody can be used to detect GPCRX protein (e.g., in a cellular lysate or cell supernatant) in order to evaluate the abundance and pattern of expression of the GPCRX protein. Anti-GPCRX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

GPCRX Recombinant Expression Vectors and Host Cells

Another aspect of the invention pertains to vectors, preferably expression vectors, containing a nucleic acid encoding an GPCRX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GPCRX proteins, mutant forms of GPCRX proteins, fusion proteins, etc.).

The recombinant expression vectors of the invention can be designed for expression of GPCRX proteins in prokaryotic or eukaryotic cells. For example, GPCRX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1 990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the GPCRX expression vector is a yeast expression vector. Examples of vectors for expression in yeast Saccharomyces cerivisae include pYepSec1(Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, GPCRX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufinan, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No.4,873,316 and European Application Publication No.264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to GPCRX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, GPCRX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding GPCRX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GPCRX protein. Accordingly, the invention further provides methods for producing GPCRX protein using the host cells of the invention. In one embodiment, the method comprises culturing the host cell of invention (into which a recombinant expression vector encoding GPCRX protein has been introduced) in a suitable medium such that GPCRX protein is produced. In another embodiment, the method further comprises isolating GPCRX protein from the medium or the host cell.

Transgenic GPCRX Animals

The host cells of the invention can also be used to produce non-human transgenic animals. For example, in one embodiment, a host cell of the invention is a fertilized oocyte or an embryonic stem cell into which GPCRX protein-coding sequences have been introduced. Such host cells can then be used to create non-human transgenic animals in which exogenous GPCRX sequences have been introduced into their genome or homologous recombinant animals in which endogenous GPCRX sequences have been altered. Such animals are useful for studying the function and/or activity of GPCRX protein and for identifying and/or evaluating modulators of GPCRX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GPCRX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GPCRX-encoding nucleic acid into the male pronuclei of a fertilized oocyte (e.g., by microinjection, retroviral infection) and allowing the oocyte to develop in a pseudopregnant female foster animal. The human GPCRX CDNA sequences of SEQ ID NOS: 1, 3, 5, 7, 9, 11, 13, 15, 17, 19 and 21, can be introduced as a transgene into the genome of a non-human animal. Alternatively, a non-human homologue of the human GPCRX gene, such as a mouse GPCRX gene, can be isolated based on hybridization to the human GPCRX CDNA (described further supra) and used as a transgene. Intronic sequences and polyadenylation signals can also be included in the transgene to increase the efficiency of expression of the transgene. A tissue-specific regulatory sequence(s) can be operably-linked to the GPCRX transgene to direct expression of GPCRX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the GPCRX transgene in its genome and/or expression of GPCRX mRNA in tissues or cells of the animals. A transgenic founder animal can then be used to breed additional animals carrying the transgene. Moreover, transgenic animals carrying a transgene-encoding GPCRX protein can further be bred to other transgenic animals carrying other transgenes.

To create a homologous recombinant animal, a vector is prepared which contains at least a portion of an GPCRX gene into which a deletion, addition or substitution has been introduced to thereby alter, e.g., functionally disrupt, the GPCRX gene. The GPCRX gene can be a human gene (e.g., the cDNA of SEQ ID NOS:1, 3, 5, 7, 9, 11, 13, 15, 17, 19 and 21), but more preferably, is a non-human homologue of a human GPCRX gene. For example, a mouse homologue of human GPCRX gene of SEQ ID NOS:1, 3, 5, 7, 9, 11, 13, 15, 17, 19 and 21, can be used to construct a homologous recombination vector suitable for altering an endogenous GPCRX gene in the mouse genome. In one embodiment, the vector is designed such that, upon homologous recombination, the endogenous GPCRX gene is functionally disrupted (i.e., no longer encodes a functional protein; also referred to as a “knock out” vector).

Alternatively, the vector can be designed such that, upon homologous recombination, the endogenous GPCRX gene is mutated or otherwise altered but still encodes functional protein (e.g., the upstream regulatory region can be altered to thereby alter the expression of the endogenous GPCRX protein). In the homologous recombination vector, the altered portion of the GPCRX gene is flanked at its 5′- and 3′-termini by additional nucleic acid of the GPCRX gene to allow for homologous recombination to occur between the exogenous GPCRX gene carried by the vector and an endogenous GPCRX gene in an embryonic stem cell. The additional flanking GPCRX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced GPCRX gene has homologously-recombined with the endogenous GPCRX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The GPCRX nucleic acid molecules, GPCRX proteins, and anti-GPCRX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an GPCRX protein or anti-GPCRX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express GPCRX protein (e.g., via a recombinant expression vector in a host cell in gene therapy applications), to detect GPCRX mRNA (e.g., in a biological sample) or a genetic lesion in an GPCRX gene, and to modulate GPCRX activity, as described further, below. In addition, the GPCRX proteins can be used to screen drugs or compounds that modulate the GPCRX protein activity or expression as well as to treat disorders characterized by insufficient or excessive production of GPCRX protein or production of GPCRX protein forms that have decreased or aberrant activity compared to GPCRX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-GPCRX antibodies of the invention can be used to detect and isolate GPCRX proteins and modulate GPCRX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to GPCRX proteins or have a stimulatory or inhibitory effect on, e.g., GPCRX protein expression or GPCRX protein activity. The invention also includes compounds identified in the screening assays described herein.

In one embodiment, the invention provides assays for screening candidate or test compounds which bind to or modulate the activity of the membrane-bound form of an GPCRX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of GPCRX protein, or a biologically-active portion thereof, on the cell surface is contacted with a test compound and the ability of the test compound to bind to an GPCRX protein determined. The cell, for example, can of mammalian origin or a yeast cell. Determining the ability of the test compound to bind to the GPCRX protein can be accomplished, for example, by coupling the test compound with a radioisotope or enzymatic label such that binding of the test compound to the GPCRX protein or biologically-active portion thereof can be determined by detecting the labeled compound in a complex. For example, test compounds can be labeled with ^{125I,} ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of GPCRX protein, or a biologically-active portion thereof, on the cell surface with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX protein or a biologically-active portion thereof as compared to the known compound.

In another embodiment, an assay is a cell-based assay comprising contacting a cell expressing a membrane-bound form of GPCRX protein, or a biologically-active portion thereof, on the cell surface with a test compound and determining the ability of the test compound to modulate (e.g., stimulate or inhibit) the activity of the GPCRX protein or biologically-active portion thereof. Determining the ability of the test compound to modulate the activity of GPCRX or a biologically-active portion thereof can be accomplished, for example, by determining the ability of the GPCRX protein to bind to or interact with an GPCRX target molecule. As used herein, a “target molecule” is a molecule with which an GPCRX protein binds or interacts in nature, for example, a molecule on the surface of a cell which expresses an GPCRX interacting protein, a molecule on the surface of a second cell, a molecule in the extracellular milieu, a molecule associated with the internal surface of a cell membrane or a cytoplasmic molecule. An GPCRX target molecule can be a non-GPCRX molecule or an GPCRX protein or polypeptide of the invention. In one embodiment, an GPCRX target molecule is a component of a signal transduction pathway that facilitates transduction of an extracellular signal (e.g. a signal generated by binding of a compound to a membrane-bound GPCRX molecule) through the cell membrane and into the cell. The target, for example, can be a second intercellular protein that has catalytic activity or a protein that facilitates the association of downstream signaling molecules with GPCRX.

Determining the ability of the GPCRX protein to bind to or interact with an GPCRX target molecule can be accomplished by one of the methods described above for determining direct binding. In one embodiment, determining the ability of the GPCRX protein to bind to or interact with an GPCRX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an GPCRX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an GPCRX protein or biologically-active portion thereof with a test compound and determining the ability of the test compound to bind to the GPCRX protein or biologically-active portion thereof. Binding of the test compound to the GPCRX protein can be determined either directly or indirectly as described above. In one such embodiment, the assay comprises contacting the GPCRX protein or biologically-active portion thereof with a known compound which binds GPCRX to form an assay mixture, contacting the assay mixture with a test compound, and determining the ability of the test compound to interact with an GPCRX protein, wherein determining the ability of the test compound to interact with an GPCRX protein comprises determining the ability of the test compound to preferentially bind to GPCRX or biologically-active portion thereof as compared to the known compound.

In still another embodiment, an assay is a cell-free assay comprising contacting GPCRX protein or biologically-active portion thereof with a test compound and determining the ability of the test compound to modulate (e.g. stimulate or inhibit) the activity of the GPCRX protein or biologically-active portion thereof Determining the ability of the test compound to modulate the activity of GPCRX can be accomplished, for example, by determining the ability of the GPCRX protein to bind to an GPCRX target molecule by one of the methods described above for determining direct binding. In an alternative embodiment, determining the ability of the test compound to modulate the activity of GPCRX protein can be accomplished by determining the ability of the GPCRX protein further modulate an GPCRX target molecule. For example, the catalytic/enzymatic activity of the target molecule on an appropriate substrate can be determined as described, supra.

In yet another embodiment, the cell-free assay comprises contacting the GPCRX protein or biologically-active portion thereof with a known compound which binds GPCRX protein to form an assay mixture, contacting the assay mixture with a test compound, and determining the ability of the test compound to interact with an GPCRX protein, wherein determining the ability of the test compound to interact with an GPCRX protein comprises determining the ability of the GPCRX protein to preferentially bind to or modulate the activity of an GPCRX target molecule.

The cell-free assays of the invention are amenable to use of both the soluble form or the membrane-bound form of GPCRX protein. In the case of cell-free assays comprising the membrane-bound form of GPCRX protein, it may be desirable to utilize a solubilizing agent such that the membrane-bound form of GPCRX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)s, N-dodecyl--N,N-dimethyl-3-ammonio- 1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either GPCRX protein or its target molecule to facilitate separation of complexed from uncomplexed forms of one or both of the proteins, as well as to accommodate automation of the assay. Binding of a test compound to GPCRX protein, or interaction of GPCRX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-GPCRX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or GPCRX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of GPCRX protein binding or activity determined using standard techniques.

Other techniques for immobilizing proteins on matrices can also be used in the screening assays of the invention. For example, either the GPCRX protein or its target molecule can be immobilized utilizing conjugation of biotin and streptavidin. Biotinylated GPCRX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with GPCRX protein or target molecules, but which do not interfere with binding of the GPCRX protein to its target molecule, can be derivatized to the wells of the plate, and unbound target or GPCRX protein trapped in the wells by antibody conjugation. Methods for detecting such complexes, in addition to those described above for the GST-immobilized complexes, include immunodetection of complexes using antibodies reactive with the GPCRX protein or target molecule, as well as enzyme-linked assays that rely on detecting an enzymatic activity associated with the GPCRX protein or target molecule.

In another embodiment, modulators of GPCRX protein expression are identified in a method wherein a cell is contacted with a candidate compound and the expression of GPCRX mRNA or protein in the cell is determined. The level of expression of GPCRX mRNA or protein in the presence of the candidate compound is compared to the level of expression of GPCRX mRNA or protein in the absence of the candidate compound. The candidate compound can then be identified as a modulator of GPCRX mRNA or protein expression based upon this comparison. For example, when expression of GPCRX mRNA or protein is greater (i.e., statistically significantly greater) in the presence of the candidate compound than in its absence, the candidate compound is identified as a stimulator of GPCRX mRNA or protein expression. Alternatively, when expression of GPCRX mRNA or protein is less (statistically significantly less) in the presence of the candidate compound than in its absence, the candidate compound is identified as an inhibitor of GPCRX mRNA or protein expression. The level of GPCRX mRNA or protein expression in the cells can be determined by methods described herein for detecting GPCRX mRNA or protein.

In yet another aspect of the invention, the GPCRX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with GPCRX (“GPCRX-binding proteins” or “GPCRX-bp”) and modulate GPCRX activity. Such GPCRX-binding proteins are also likely to be involved in the propagation of signals by the GPCRX proteins as, for example, upstream or downstream elements of the GPCRX pathway.

The two-hybrid system is based on the modular nature of most transcription factors, which consist of separable DNA-binding and activation domains. Briefly, the assay utilizes two different DNA constructs. In one construct, the gene that codes for GPCRX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an GPCRX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with GPCRX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the GPCRX sequences, SEQ ID NOS:1, 3, 5, 7, 9, 11, 13, 15, 17, 19 and 21, or fragments or derivatives thereof, can be used to map the location of the GPCRX genes, respectively, on a chromosome. The mapping of the GPCRX sequences to chromosomes is an important first step in correlating these sequences with genes associated with disease.

Briefly, GPCRX genes can be mapped to chromosomes by preparing PCR primers (preferably 15-25 bp in length) from the GPCRX sequences. Computer analysis of the GPCRX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the GPCRX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GPCRX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the GPCRX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The GPCRX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the GPCRX sequences described herein can be used to prepare two PCR primers from the 5 - and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The GPCRX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19 and 21, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining GPCRX protein and/or nucleic acid expression as well as GPCRX activity, in the context of a biological sample (e.g., blood, serum, cells, tissue) to thereby determine whether an individual is afflicted with a disease or disorder, or is at risk of developing a disorder, associated with aberrant GPCRX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with GPCRX protein, nucleic acid expression or activity. For example, mutations in an GPCRX gene can be assayed in a biological sample. Such assays can be used for prognostic or predictive purpose to thereby prophylactically treat an individual prior to the onset of a disorder characterized by or associated with GPCRX protein, nucleic acid expression, or biological activity.

Another aspect of the invention provides methods for determining GPCRX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of GPCRX in clinical trials.

These and other agents are described in further detail in the following sections.

Diagnostic Assays

An exemplary method for detecting the presence or absence of GPCRX in a biological sample involves obtaining a biological sample from a test subject and contacting the biological sample with a compound or an agent capable of detecting GPCRX protein or nucleic acid (e.g., mRNA, genomic DNA) that encodes GPCRX protein such that the presence of GPCRX is detected in the biological sample. An agent for detecting GPCRX mRNA or genomic DNA is a labeled nucleic acid probe capable of hybridizing to GPCRX mRNA or genomic DNA. The nucleic acid probe can be, for example, a full-length GPCRX nucleic acid, such as the nucleic acid of SEQ ID NOS:1, 3, 5, 7, 9, 11, 13, 15, 17, 19 and 21, or a portion thereof, such as an oligonucleotide of at least 15, 30, 50, 100, 250 or 500 nucleotides in length and sufficient to specifically hybridize under stringent conditions to GPCRX mRNA or genomic DNA. Other suitable probes for use in the diagnostic assays of the invention are described herein.

An agent for detecting GPCRX protein is an antibody capable of binding to GPCRX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect GPCRX mRNA, protein, or genomic DNA in a biological sample in vitro as well as in vivo. For example, in vitro techniques for detection of GPCRX mRNA include Northern hybridizations and in situ hybridizations. In vitro techniques for detection of GPCRX protein include enzyme linked immunosorbent assays (ELISAs), Western blots, immunoprecipitations, and immunofluorescence. In vitro techniques for detection of GPCRX genomic DNA include Southern hybridizations. Furthermore, in vivo techniques for detection of GPCRX protein include introducing into a subject a labeled anti-GPCRX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GPCRX protein, mRNA, or genomic DNA, such that the presence of GPCRX protein, mRNA or genomic DNA is detected in the biological sample, and comparing the presence of GPCRX protein, mRNA or genomic DNA in the control sample with the presence of GPCRX protein, mRNA or genomic DNA in the test sample.

The invention also encompasses kits for detecting the presence of GPCRX in a biological sample. For example, the kit can comprise: a labeled compound or agent capable of detecting GPCRX protein or mRNA in a biological sample; means for determining the amount of GPCRX in the sample; and means for comparing the amount of GPCRX in the sample with a standard. The compound or agent can be packaged in a suitable container. The kit can further comprise instructions for using the kit to detect GPCRX protein or nucleic acid.

Prognostic Assays

The diagnostic methods described herein can furthermore be utilized to identify subjects having or at risk of developing a disease or disorder associated with aberrant GPCRX expression or activity. For example, the assays described herein, such as the preceding diagnostic assays or the following assays, can be utilized to identify a subject having or at risk of developing a disorder associated with GPCRX protein, nucleic acid expression or activity. Alternatively, the prognostic assays can be utilized to identify a subject having or at risk for developing a disease or disorder. Thus, the invention provides a method for identifying a disease or disorder associated with aberrant GPCRX expression or activity in which a test sample is obtained from a subject and GPCRX protein or nucleic acid (e.g., mRNA, genomic DNA) is detected, wherein the presence of GPCRX protein or nucleic acid is diagnostic for a subject having or at risk of developing a disease or disorder associated with aberrant GPCRX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant GPCRX expression or activity. For example, such methods can be used to determine whether a subject can be effectively treated with an agent for a disorder. Thus, the invention provides methods for determining whether a subject can be effectively treated with an agent for a disorder associated with aberrant GPCRX expression or activity in which a test sample is obtained and GPCRX protein or nucleic acid is detected (e.g., wherein the presence of GPCRX protein or nucleic acid is diagnostic for a subject that can be administered the agent to treat a disorder associated with aberrant GPCRX expression or activity).

The methods of the invention can also be used to detect genetic lesions in an GPCRX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an GPCRX-protein, or the misexpression of the GPCRX gene. For example, such genetic lesions can be detected by ascertaining the existence of at least one of: (i) a deletion of one or more nucleotides from an GPCRX gene; (ii) an addition of one or more nucleotides to an GPCRX gene; (iii) a substitution of one or more nucleotides of an GPCRX gene, (iv) a chromosomal rearrangement of an GPCRX gene; (v) an alteration in the level of a messenger RNA transcript of an GPCRX gene, (vi) aberrant modification of an GPCRX gene, such as of the methylation pattern of the genomic DNA, (vii) the presence of a non-wild-type splicing pattern of a messenger RNA transcript of an GPCRX gene, (viii) a non-wild-type level of an GPCRX protein, (ix) allelic loss of an GPCRX gene, and (x) inappropriate post-translational modification of an GPCRX protein. As described herein, there are a large number of assay techniques known in the art which can be used for detecting lesions in an GPCRX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the GPCRX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an GPCRX gene under conditions such that hybridization and amplification of the GPCRX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an GPCRX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in GPCRX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in GPCRX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GPCRX gene and detect mutations by comparing the sequence of the sample GPCRX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the GPCRX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type GPCRX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GPCRX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an GPCRX sequence, e.g., a wild-type GPCRX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Patent No. 5,459,039.

In other embodiments, alterations in electrophoretic mobility will be used to identify mutations in GPCRX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appi. 9: 73-79. Single-stranded DNA fragments of sample and control GPCRX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g. Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an GPCRX gene.

Furthermore, any cell type or tissue, preferably peripheral blood leukocytes, in which GPCRX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on GPCRX activity (e.g., GPCRX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmnacogenomics can further be used to determine appropriate dosages and therapeutic regimens. Accordingly, the activity of GPCRX protein, expression of GPCRX nucleic acid, or mutation content of GPCRX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of GPCRX protein, expression of GPCRX nucleic acid, or mutation content of GPCRX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an GPCRX modulator, such as a modulator identified by one of the exemplary screening assays described herein.

Monitoring of Effects During Clinical Trials

Monitoring the influence of agents (e.g., drugs, compounds) on the expression or activity of GPCRX (e.g., the ability to modulate aberrant cell proliferation and/or differentiation) can be applied not only in basic drug screening, but also in clinical trials. For example, the effectiveness of an agent determined by a screening assay as described herein to increase GPCRX gene expression, protein levels, or upregulate GPCRX activity, can be monitored in clinical trails of subjects exhibiting decreased GPCRX gene expression, protein levels, or downregulated GPCRX activity. Alternatively, the effectiveness of an agent determined by a screening assay to decrease GPCRX gene expression, protein levels, or downregulate GPCRX activity, can be monitored in clinical trails of subjects exhibiting increased GPCRX gene expression, protein levels, or upregulated GPCRX activity. In such clinical trials, the expression or activity of GPCRX and, preferably, other genes that have been implicated in, for example, a cellular proliferation or immune disorder can be used as a “read out” or markers of the immune responsiveness of a particular cell.

By way of example, and not of limitation, genes, including GPCRX, that are modulated in cells by treatment with an agent (e.g., compound, drug or small molecule) that modulates GPCRX activity (e.g., identified in a screening assay as described herein) can be identified. Thus, to study the effect of agents on cellular proliferation disorders, for example, in a clinical trial, cells can be isolated and RNA prepared and analyzed for the levels of expression of GPCRX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of GPCRX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an GPCRX protein, mRNA, or genomic DNA in the preadministration sample; (iii) obtaining one or more post-administration samples from the subject; (iv) detecting the level of expression or activity of the GPCRX protein, mRNA, or genomic DNA in the post-administration samples; (v) comparing the level of expression or activity of the GPCRX protein, mRNA, or genomic DNA in the pre-administration sample with the GPCRX protein, mRNA, or genomic DNA in the post administration sample or samples; and (vi) altering the administration of the agent to the subject accordingly. For example, increased administration of the agent may be desirable to increase the expression or activity of GPCRX to higher levels than detected, i.e., to increase the effectiveness of the agent. Alternatively, decreased administration of the agent may be desirable to decrease expression or activity of GPCRX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant GPCRX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderna, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant GPCRX expression or activity, by administering to the subject an agent that modulates GPCRX expression or at least one GPCRX activity. Subjects at risk for a disease that is caused or contributed to by aberrant GPCRX expression or activity can be identified by, for example, any or a combination of diagnostic or prognostic assays as described herein. Administration of a prophylactic agent can occur prior to the manifestation of symptoms characteristic of the GPCRX aberrancy, such that a disease or disorder is prevented or, alternatively, delayed in its progression. Depending upon the type of GPCRX aberrancy, for example, an GPCRX agonist or GPCRX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating GPCRX expression or activity for therapeutic purposes. The modulatory method of the invention involves contacting a cell with an agent that modulates one or more of the activities of GPCRX protein activity associated with the cell. An agent that modulates GPCRX protein activity can be an agent as described herein, such as a nucleic acid or a protein, a naturally-occurring cognate ligand of an GPCRX protein, a peptide, an GPCRX peptidomimetic, or other small molecule. In one embodiment, the agent stimulates one or more GPCRX protein activity. Examples of such stimulatory agents include active GPCRX protein and a nucleic acid molecule encoding GPCRX that has been introduced into the cell. In another embodiment, the agent inhibits one or more GPCRX protein activity. Examples of such inhibitory agents include antisense GPCRX nucleic acid molecules and anti-GPCRX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an GPCRX protein or nucleic acid molecule. In one embodiment, the method involves administering an agent (e.g., an agent identified by a screening assay described herein), or combination of agents that modulates (e.g., up-regulates or down-regulates) GPCRX expression or activity. In another embodiment, the method involves administering an GPCRX protein or nucleic acid molecule as therapy to compensate for reduced or aberrant GPCRX expression or activity.

Stimulation of GPCRX activity is desirable in situations in which GPCRX is abnormally downregulated and/or in which increased GPCRX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The GPCRX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the GPCRX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the GPCRX protein, and the GPCRX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

### EXAMPLES

The following examples illustrate by way of non-limiting example various aspects of the invention.

The following examples illustrate by way of non-limiting example various aspects of the invention.

### Example 1

### Method of Identifying the Nucleic Acids

The novel nucleic acids of the invention were identified by TblastN using a proprietary sequence file, run against the Genomic Daily Files made available by GenBank. The nucleic acids were further predicted by the proprietary software program GenScan™, including selection of exons. These were further modified by means of similarities using BLAST searches. The sequences were then manually corrected for apparent inconsistencies, thereby obtaining the sequences encoding the full-length proteins.

### Example 2

### Quantitative expression analysis of GPCR2 in various cells and tissues

The quantitative expression of clone GPCR1 was assessed in a large number of normal and tumor sample cells and cell lines (Panel 1), as well as in surgical tissue samples (Panel 2), by real time quantitative PCR (TaqMan®) performed on a Perkin-Elmer Biosystems ABI PRISM® 7700 Sequence Detection System.

First, 96 RNA samples were normalized to β-actin and GAPDH. RNA (˜50 ng total or ˜1 ng polyA+) was converted to cDNA using the TaqMan® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TaqMan® reaction using β-actin and GAPDH TaqMan® Assay Reagents (PE Biosystems; Catalog Nos. 4310881E and 4310884E, respectively) and TaqMan® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TaqMan® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tₘ=59° C., maximum primer difference=2° C., probe does not have 5′G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1X TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

The results for various cells and cell lines that constitute Panel 1 are shown in Table 10. In Table 10, the following abbreviations are used: ca.=carcinoma; *=established from metastasis; met =metastasis; s cell var=small cell variant; non-s=non-sm=non-small; squam=squamous; pl. eff=pl effusion=pleural effusion; glio=glioma; astro=astrocytoma; and neuro=neuroblastoma.

Panel 2 consists of a 96 well plate (2 control wells, 94 test samples) composed of RNA/cDNA isolated from human tissue procured by surgeons working in close cooperation with the National Cancer Institute's Cooperative Human Tissue Network (CHTN) or the National Disease Research Initiative (NDRI). The tissues procured are derived from human malignancies and in cases where indicated many malignant tissues have “matched margins”. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Tables 10 and 11). In addition, RNA/cDNA was obtained from various human tissues derived from human autopsies performed on deceased elderly people or sudden death victims (accidents, etc.). These tissue were ascertained to be free of disease and were purchased from various high quality commercial sources such as Clontech, Research Genetics, and Invitrogen.

RNA integrity from all samples is controlled for quality by visual assessment of agarose gel electrophoresis using 28s and 18s ribosomal RNA staining intensity ratio as a guide (2:1 to 2.5:1 28s: 18s) and the presence of low molecular weight RNAs indicative of degradation products. Samples are quality controlled for genomic DNA contamination by reactions run in the absence of reverse transcriptase using probe and primer sets designed to amplify across the span of a single exon.

### Example 3

### Quantitative expression analysis of GPCR1 in various cells and tissues.

The quantitative expression of GPCR1 was assessed in a large number of normal and tumor sample cells and cell lines (Panel 1), as well as in surgical tissue samples (Panel 2), by real time quantitative PCR (TaqMan®) performed on a Perkin-Elmer Biosystems ABI PRISM®) 7700 Sequence Detection System as described above in Example 2, with the following primers (Table 9).

TABLE 9
Probe set Ag2695.

| | | | | | |
|---|---|---|---|---|---|
| | | | | Start | SEQ |
| Primers | Sequences | Tₘ | Length | Pos. | ID NO: |

| | | | | | |
|---|---|---|---|---|---|
| Forward | 5′-GGGAAATGGGTTGTCCATATAT-3′ | 58.8 | 22 | 266 | 63 |
| Probe | FAM-5-TCCTGCAGCCTTATAAGAAGTCCACA-3′-TAMRA | 66.2 | 26 | 292 | 64 |
| Reverse | 5′-ATCTGAAATGGCCAGATTTAGC-3 | 59.6 | 22 | 335 | 65 |

The TaqMan results for panels 1 and 2 are shown in Table 10.

TABLE 10
TaqMan results for GPCR1

| | Run 1 | Run 2 | | Run 1 | Run 2 |
|---|---|---|---|---|---|
| | Rel | Rel. | | Rel. | Rel. |
| Tissue_Name | Expr. % | Expr. % | Tissue_Name | Expr. % | Expr. % |
| Liver adenocarcinoma | 0 | 0 | Normal Colon GENPAK 061003 | 3.82 | 3.28 |
| Pancreas | 2.15 | 4.21 | 83220 CC NAT (ODO3866) | 2.74 | 0.51 |
| Pancreatic ca. CAPAN 2 | 0 | 0 | 83221 CC Gr.2 rectosigmoid (ODO3868) | 0.13 | 0.21 |
| Adrenal gland | 67.36 | 100 | 83222 CC NAT (ODO3868) | 0.92 | 0.27 |
| Thyroid | 5.08 | 1.96 | 83235 CC Mod Diff (ODO3920) | 0.65 | 0.22 |
| Salivary gland | 5.11 | 3.54 | 83236 CC NAT (ODO3920) | 0.46 | 0.28 |
| Pituitary gland | 1.05 | 4.9 | 83237 CC Gr.2 ascend colon (ODO3921) | 3.21 | 2.24 |
| Brain (fetal) | 3.49 | 0 | 83238 CC NAT (ODO3921) | 0.81 | 0.59 |
| Brain (whole) | 20.31 | 16.04 | 83241 CC from Partial Hepatectomy | 1.58 | 2.01 |
| | | | (ODO4309) | | |
| Brain (amygdala) | 22.22 | 12.5 | 83242 Liver NAT (ODO4309) | 1.26 | 1.47 |
| Brain (cerebellum) | 2.15 | 4.54 | 87472 Colon mets to lung (OD04451-01) | 0.3 | 1.23 |
| Brain (hippocampus) | 61.13 | 35.11 | 87473 Lung NAT (OD04451-02) | 2.88 | 1.71 |
| Brain (thalamus) | 8.72 | 18.56 | Normal Prostate Clontech A + 6546-1 | 2.01 | 0.77 |
| Cerebral Cortex | 45.06 | 53.22 | 84140 Prostate Cancer (OD04410) | 1.88 | 1.72 |
| Spinal cord | 11.34 | 7.97 | 84141 Prostate NAT (OD04410) | 3.52 | 3.9 |
| CNS ca. (glio/astro) U87-MG | 0 | 0 | 87073 Prostate Cancer (OD04720-01) | 1.73 | 0.5 |
| CNS ca. (glio/astro) U-118-MG | 4.21 | 0 | 87074 Prostate NAT (OD04720-02) | 5.01 | 4.42 |
| CNS ca. (astro) SW 1783 | 0 | 0 | Normal Lung GENPAK 061010 | 6.93 | 7.8 |
| CNS ca.* (neuro; met) SK-N-AS | 0 | 0 | 83239 Lung Met to Muscle (ODO4286) | 2.16 | 1.98 |
| CNS ca. (astro) SF-539 | 8.96 | 0 | 83240 Muscle NAT (ODO4286) | 0.3 | 0.51 |
| CNS ca. (astro) SNB-75 | 0 | 0 | 84136 Lung Malignant Cancer (OD03126) | 2.4 | 3.69 |
| CNS ca. (glio) SNB-19 | 0 | 2.86 | 84137 Lung NAT (OD03126) | 6.93 | 6.7 |
| CNS ca. (glio) U251 | 0 | 0 | 84871 Lung Cancer (OD04404) | 5.48 | 2.12 |
| CNS ca. (glio) SF-295 | 0 | 1.96 | 84872 Lung NAT (OD04404) | 1.55 | 1.91 |
| Heart | 41.47 | 32.99 | 84875 Lung Cancer (OD04565) | 0.59 | 0.45 |
| Skeletal muscle | 0 | 0 | 85950 Lung Cancer (OD04237-01) | 4.54 | 3.77 |
| Bone marrow | 3.49 | 11.74 | 85970 Lung NAT (OD04237-02) | 3.93 | 5.18 |
| Thymus | 1.44 | 6.79 | 83255 Ocular Mel Met to Liver (ODO4310) | 0 | 0.16 |
| Spleen | 100 | 59.05 | 83256 Liver NAT (OD04310) | 1.72 | 0.69 |
| Lymph node | 32.31 | 26.79 | 84139 Melanoma Mets to Lung (OD04321) | 100 | 100 |
| Colorectal | 17.92 | 20.17 | 84138 Lung NAT (OD04321) | 7.33 | 6.52 |
| Stomach | 9.54 | 0 | Normal Kidney GENPAK 061008 | 6.38 | 5.04 |
| Small intestine | 13.4 | 38.96 | 83786 Kidney Ca, Nuclear grade 2 | 34.15 | 33.92 |
| | | | (OD04338) | | |
| Colon ca. SW480 | 0 | 0 | 83787 Kidney NAT (OD04338) | 5.18 | 5.63 |
| Colon ca.* (SW480 met) SW620 | 0 | 0 | 83788 Kidney Ca Nuclear grade ½ | 1.39 | 0.78 |
| | | | (OD04339) | | |
| Colon Ca. HT29 | 0 | 1.9 | 83789 Kidney NAT (OD04339) | 0.95 | 2.02 |
| Colon ca. HCT-116 | 0 | 0 | 83790 Kidney Ca, Clear cell type (OD04340) | 8.54 | 10.29 |
| Colon Ca. CaCo-2 | 0 | 0 | 83791 Kidney NAT (OD04340) | 11.34 | 6.56 |
| 83219 CC Well to Mod Diff | 10.96 | 3.37 | 83792 Kidney Ca, Nuclear grade 3 | 1.44 | 1.94 |
| (ODO3866) | | | (OD04348) | | |
| Colon ca. HCC-2998 | 0.62 | 0 | 83793 Kidney NAT (OD04348) | 5.37 | 7.33 |
| Gastric ca.* (liver met) NCI-N87 | 0 | 1.9 | 87474 Kidney Cancer (OD04622-01) | 48.97 | 63.73 |
| Bladder | 0 | 2.42 | 87475 Kidney NAT (OD04622-03) | 0.73 | 1.16 |
| Trachea | 4.15 | 0 | 85973 Kidney Cancer (OD04450-01) | 1.38 | 1.42 |
| Kidney | 2.37 | 0 | 85974 Kidney NAT(ODO4450-03) | 5.71 | 4.64 |
| Kidney (fetal) | 0 | 3.93 | Kidney Cancer Clontech 8120607 | 0.2 | 0 |
| Renal ca. 786-0 | 0 | 0 | Kidney NAT Clontech 8120608 | 1.35 | 0.55 |
| Renal ca. A498 | 0 | 1.91 | Kidney Cancer Clontech 8120613 | 0.12 | 0.1 |
| Renal ca. RXF 393 | 0 | 0 | Kidney NAT Clontech 8120614 | 0.61 | 0.04 |
| Renal ca. ACHN | 0 | 0 | Kidney Cancer Clontech 9010320 | 3.96 | 2.47 |
| Renal ca. UO-31 | 0 | 0 | Kidney NAT Clontech 9010321 | 0.45 | 1.12 |
| Renal ca. TK-10 | 0 | 0 | Normal Uterus GENPAK 061018 | 0.41 | 0.32 |
| Liver | 1.54 | 3.93 | Uterus Cancer GENPAK 064011 | 1.23 | 0.94 |
| Liver (fetal) | 0 | 2.47 | Normal Thyroid Clontech A + 6570-1 | 0.58 | 0.81 |
| Liver ca. (hepatoblast) HepG2 | 0 | 0 | Thyroid Cancer GENPAK 064010 | 7.28 | 5.71 |
| Lung | 18.82 | 16.15 | Thyroid Cancer INVITROGEN A302152 | 7.08 | 8.9 |
| Lung (fetal) | 3.72 | 3.69 | Thyroid NAT INVITROGEN A302153 | 1.3 | 1.07 |
| Lung ca. (small cell) LX-1 | 0 | 0 | Normal Breast GENPAK 061019 | 1.14 | 1.83 |
| Lung ca. (small cell) NCI-H69 | 0 | 0 | 84877 Breast Cancer (OD04566) | 0.19 | 1.33 |
| Lung ca. (s. cell var.) SHP-77 | 0 | 0 | 85975 Breast Cancer (OD04590-01) | 2.24 | 2.03 |
| Lung ca. (large cell) NCI-H460 | 0 | 0 | 85976 Breast Cancer Mets (OD04590-03) | 6.75 | 8.48 |
| Lung ca. (non-sm. cell) A549 | 0 | 0 | 87070 Breast Cancer Metastasis (OD04655-05) | 2.66 | 2.66 |
| Lung ca. (non-s. cell) NCI-H23 | 0 | 1.81 | GENPAK Breast Cancer 064006 | 0.64 | 0.55 |
| Lung ca (non-s. cell) HOP-62 | 0 | 0 | Breast Cancer Clontech 9100266 | 0.13 | 0.26 |
| Lung ca. (non-s. cl) NCI-H522 | 0 | 0 | Breast NAT Clontech 9100265 | 0.22 | 0.54 |
| Lung ca. (squam.) SW 900 | 0 | 0 | Breast Cancer INVITROGEN A209073 | 2.29 | 1.73 |
| Lung ca. (squam.) NCI-H596 | 2.3 | 0 | Breast NAT INVITROGEN A2090734 | 0.36 | 0.28 |
| Mammary gland | 5.18 | 9.02 | Normal Liver GENPAK 061009 | 0.63 | 0.22 |
| Breast ca.* (pl. effusion) MCF-7 | 0 | 0 | Liver Cancer GENPAK 064003 | 0.78 | 0 |
| Breast ca.* (pl. ef) MDA-MB-231 | 0 | 0 | Liver Cancer Research Genetics RNA 1025 | 0.22 | 0.15 |
| Breast ca.* (pl. effusion) T47D | 0 | 0 | Liver Cancer Research Genetics RNA 1026 | 0.85 | 0.63 |
| Breast ca. BT-549 | 0 | 0 | Paired Liver Cancer Tissue Research Genetics | 0.36 | 0.52 |
| | | | RNA 6004-T | | |
| Breast ca. MDA-N | 0 | 0 | Paired Liver Tissue Research Genetics RNA | 0.87 | 0.34 |
| | | | 6004-N | | |
| Ovary | 16.15 | 9.09 | Paired Liver Cancer Tissue Research Genetics | 0.23 | 0.23 |
| | | | RNA 6005-T | | |
| Ovarian ca. OVCAR-3 | 0 | 0 | Paired Liver Tissue Research Genetics RNA | 0.33 | 0.28 |
| | | | 6005-N | | |
| Ovarian ca. OVCAR-4 | 0 | 0 | Normal Bladder GENPAK 061001 | 0.94 | 1.5 |
| Ovarian ca. OVCAR-5 | 0 | 0 | Bladder Cancer Research Genetics RNA 1023 | 0.11 | 0.38 |
| Ovarian ca. OVCAR-8 | 0 | 0 | Bladder Cancer INVITROGEN A302173 | 0.54 | 1.22 |
| Ovarian ca. IGROV-1 | 0 | 0 | 87071 Bladder Cancer (OD04718-01) | 1.82 | 1.15 |
| Ovarian ca.* (ascites) SK-OV-3 | 0 | 0 | 87072 Bladder Normal Adjacent (OD04718-03) | 2.18 | 1.94 |
| Uterus | 6.47 | 4.3 | Normal Ovary Res. Gen. | 0.62 | 0.19 |
| Plancenta | 48.97 | 32.53 | Ovarian Cancer GENPAK 064008 | 2.92 | 1.67 |
| Prostate | 14.97 | 2.19 | 87492 Ovary Cancer (OD04768-07) | 14.36 | 10.81 |
| Prostate ca.* (bone met) PC-3 | 0 | 0 | 87493 Ovary NAT (OD04768-08) | 1.37 | 0.96 |
| Testis | 2.37 | 11.03 | Normal Stomach GENPAK 061017 | 0.59 | 1.5 |
| Melanoma Hs688 (A).T | 0 | 0 | NAT Stomach Clontech 9060359 | 0.65 | 0.56 |
| Melanoma* (met) Hs688 (B).T | 0 | 0 | Gastric Cancer Clontech 9060395 | 1.06 | 1.22 |
| Melanoma UACC-62 | 0 | 0 | NAT Stomach Clontech 9060394 | 1.15 | 0.44 |
| Melanoma M14 | 0 | 0 | Gastric Cancer Clontech 9060397 | 0.94 | 0.92 |
| Melanoma LOX IMV1 | 0 | 0 | NAT Stomach Clontech 9060396 | 1.71 | 1.14 |
| Melanoma* (met) SK-MEL-5 | 3.98 | 4.27 | Gastric Cancer GENPAK 064005 | 3 | 2.57 |
| Adipose | 15.5 | 16.72 | | | |

The quantitative expression of GPCR1 was also assessed using microtiter plates containing RNA samples from a variety of normal and pathology-derived cells, cell lines and tissues using real time quantitative PCR (RTQ PCR; TaqMan®). RTQ PCR was performed on a Perkin-Elmer Biosystems ABI PRISM® 7700 Sequence Detection System. In this Example, samples are referred to as Panel 4 and contain cells and cell lines from normal cells and cells related to inflammatory conditions.

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were freshly prepared from normal human blood using standard methods known in the art. Monocytes were isolated and differentiated by methods well known in the art. CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and +ve selection. Then CD45RO beads were used to isolate the CD45RO CD4 lymphocytes with the remaining cells being CD45RA CD4 lymphocytes. To obtain B cells, tonsils were procured from NDRI and dissected to isolate B cells which were activated using PWM at 5 μg/ml or anti-CD40 (Pharmingen) at approximately 10 μg/ml and IL-4 at 5-10 ng/ml. Primary and secondary Th1/Th2 and Tr1 cells were cultured using a standard method well known in the art. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture: keratinocyte line CCD106 and an airway epithelial tumor line NCI-H292 were also obtained from the ATCC. CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

The primer-probe set used for expression analysis of clone GPCR1 is shown in Table 9.

The results of two replicate runs assessing the expression of GPCR1 on Panel 3 are shown in Table 11. GPCR1 is expressed in normal tissues, such as kidney, thymus, lung and colon. It is most highly expressed on resting monocytes. Surprising results relating to inflammation indicate that the expression of GPCR1 is reduced greater than 100 fold (virtually eliminated) on immune-activated monocytes.

TABLE 11
TaqMan results for clone GPCR1 on Panel 3.

| | Rel. | Rel. | | Rel. | Rel. |
|---|---|---|---|---|---|
| Tissue_Name | Expr. % | Expr. % | Tissue_Name | Expr. % | Expr. % |
| 93768_Secondary Th1_anti- | 1.0 | 1.6 | 93100_HUVEC (Endothelial)_IL-1b | 0.0 | 0.0 |
| CD28/anti-CD3 | | | | | |
| 93769_Secondary Th2_anti- | 21.0 | 29.5 | 93779_HUVEC (Endothelial)_IFN | 0.0 | 0.0 |
| CD28/anti-CD3 | | | gamma | | |
| 93770_Secondary Th1_anti- | 5.0 | 10.8 | 93102_HUVEC (Endothelial)_TNF | 0.0 | 0.0 |
| CD28/anti-CD3 | | | alpha + IFN gamma | | |
| 93573_Secondary Th1_resting day 4-6 | 0.7 | 3.8 | 93101_HUVEC (Endothelial)_TNF | 0.0 | 0.0 |
| in IL-2 | | | alpha + 1L4 | | |
| 93572_Secondary Th2_resting day 4-6 | 13.9 | 15.4 | 93781_HUVEC (Endothelial)_IL-11 | 0.0 | 0.0 |
| in IL-2 | | | | | |
| 93571_Secondary Th1_resting day 4-6 | 6.1 | 5.0 | 93583_Lung Microvascular | 0.0 | 0.0 |
| in IL-2 | | | Endothelial Cells_none | | |
| 93568_primary Th1_anti-CD28/anti- | 0.0 | 2.6 | 93584_Lung Microvascular | 0.0 | 0.0 |
| CD3 | | | Endothelial Cells_TNFa (4 ng/ml) and | | |
| | | | IL1b (1 ng/ml) | | |
| 93569_primary Th2_anti-CD28/anti- | 39.0 | 54.3 | 92662_Microvascular Dermal | 0.0 | 0.0 |
| CD3 | | | endothelium_none | | |
| 93570_primary Th1_anti-CD28/anti- | 10.7 | 21.6 | 92663_Microsvasular Dermal | 0.0 | 0.0 |
| CD3 | | | endothelium_TNFa (4 ng/ml) and | | |
| | | | IL1b (1 ng/ml) | | |
| 93565_primary Th1_resting day 4-6 in | 21.0 | 24.7 | 93773_Bronchial epithelium_TNFa (4 | 0.0 | 0.0 |
| IL-2 | | | ng/ml) and IL1b (1 ng/ml)** | | |
| 93566_primary Th2_resting dy 4-6 in | 26.1 | 21.2 | 93347_Small Airway | 0.0 | 0.0 |
| IL-2 | | | Epithelium_none | | |
| 93567_primary Th1_resting dy 4-6 in | 11.7 | 19.1 | 93348_Small Airway | 0.0 | 0.0 |
| IL-2 | | | Epithelium_TNFa (4 ng/ml) and IL1b | | |
| | | | (1 ng/ml) | | |
| 93351_CD45RA CD4 | 9.6 | 7.1 | 92668_Coronery Artery SMC_resting | 0.0 | 0.0 |
| lymphocyte_anti-CD28/anti-CD3 | | | | | |
| 93352_CD45RO CD4 | 15.6 | 17.3 | 92669_Coronery Artery SMC_TNFa | 0.0 | 0.0 |
| lymphocyte anti-CD28/anti-CD3 | | | (4 ng/ml) and IL1b (1 ng/ml) | | |
| 93251_CD8_Lymphocytes anti- | 11.7 | 9.3 | 93107_astrocytes_resting | 0.0 | 0.5 |
| CD28/anti-CD3 | | | | | |
| 93353_chronic CD8 Lymphocytes | 8.4 | 9.0 | 93108_astrocytes_TNFa (4 ng/ml) | 0.0 | 0.0 |
| 2ry_resting dy 4-6 in IL-2 | | | and IL1b (1 ng/ml) | | |
| 93574_chronic CD8 Lymphocytes | 22.2 | 18.3 | 92666_KU-812 (Basophil)_resting | 0.5 | 2.4 |
| 2ry_activated CD3/CD28 | | | | | |
| 93354_CD4_none | 18.6 | 9.1 | 92667_KU-812 | 2.9 | 1.7 |
| | | | (Basophil)_PMA/ionoycin | | |
| 93252_Secondary Th1/Th2/Tr1_anti- | 3.8 | 7.6 | 93579_CCD1106 | 0.0 | 0.7 |
| CD95 CH11 | | | (Keratinocytes)_none | | |
| 93103_LAK cells_resting | 43.2 | 32.5 | 93580_CCD1106 | 0.0 | 0.0 |
| | | | (Keratinocytes)_TNFa and IFNg** | | |
| 93788_LAK cells_IL-2 | 15.9 | 18.1 | 93791_Liver Cirrhosis | 4.8 | 6.3 |
| 93787_LAK cells_IL-2 + IL-12 | 32.3 | 30.8 | 93792_Lupus Kidney | 0.6 | 0.0 |
| 93789_LAK cells_IL-2 + IFN gamma | 62.9 | 57.8 | 93577 NCI-H292 | 0.0 | 0.0 |
| 93790_LAK cells_IL-2 + IL-18 | 39.2 | 52.1 | 93358_NCL-H292 IL-4 | 0.0 | 0.0 |
| 93104_LAK cells_PMA/ionomycin | 43.5 | 52.9 | 93360_NCI-H292_IL-9 | 0.0 | 0.0 |
| and IL-18 | | | | | |
| 93578_NK Cells IL-2_resting | 37.4 | 35.6 | 93359_NCI-H292_IL-13 | 0.0 | 0.0 |
| 93109_Mixed Lymphocyte | 26.1 | 23.0 | 93357_NCI-H292_IFN gamma | 0.0 | 0.0 |
| Reaction_Two Way MLR | | | | | |
| 93110_Mixed Lymphocyte | 17.0 | 10.7 | 93777_HPAEC_- | 0.0 | 0.5 |
| Reaction_Two Way MLR | | | | | |
| 93111_Mixed Lymphocyte | 2.2 | 7.4 | 93778_HPAEC_IL-1_beta/TNA alpha | 0.0 | 0.0 |
| Reaction_Two Way MLR | | | | | |
| 93112_Mononuclear Cells | 19.9 | 28.3 | 93254_Normal Human Lung | 0.0 | 0.0 |
| (PBMCs)_resting | | | Fibroblast_none | | |
| 93113_Mononuclear Cells | 28.3 | 31.4 | 93253_Normal Human Lung | 0.0 | 0.0 |
| (PBMCs)_PWM | | | Fibroblast_TNFa (4 ng/ml) and IL-1b | | |
| | | | (1 ng/ml) | | |
| 93114_Mononuclear Cells | 10.9 | 14.4 | 93257_Normal Human Lung | 0.0 | 0.0 |
| (PBMCs)_PHA-L | | | Fibroblast_IL-4 | | |
| 93249_Ramos (B cell)_none | 0.0 | 0.1 | 93256_Normal Human Lung | 0.0 | 0.0 |
| | | | Fibroblast_IL-9 | | |
| 93250_Ramos (B cell)_ionomycin | 0.0 | 0.0 | 93255_Normal Human Lung | 0.0 | 0.0 |
| | | | Fibroblast_IL-13 | | |
| 93349_B lymphocytes_PWM | 9.0 | 9.5 | 93258_Normal Human Lung | 0.0 | 0.0 |
| | | | Fibroblast_IFN gamma | | |
| 93350_B lymphoytes_CD40L and IL-4 | 9.5 | 11.1 | 93106_Dermal Fibroblasts | 0.0 | 0.0 |
| | | | CCD1070_resting | | |
| 92665_EOL-1 (Eosinophil)_dbcAMP | 1.1 | 0.0 | 93361_Dermal Fibroblasts | 17.1 | 20.3 |
| differentiated | | | CCD1070_TNF alpha 4 ng/ml | | |
| 93248_EOL-1 (Eosinophil)_dbcAMP/ | 3.7 | 0.6 | 93105_Dermal Fibroblasts | 0.0 | 0.0 |
| PMAionomycin | | | CCD1070_IL-1 beta 1 ng/ml | | |
| 93356_Dendritic Cells_none | 3.7 | 10.3 | 93772_dermal fibroblast_IFN gamma | 0.0 | 0.0 |
| 93355_Dendritic Cells_LPS 100 ng/ml | 6.1 | 8.1 | 93771_dermal fibroblast_IL-4 | 1.1 | 0.0 |
| 93775_Dendritic Cells_anti-CD40 | 4.9 | 3.6 | 93259_IBD Colitis 1** | 0.6 | 3.3 |
| 93774_Monocytes_resting | 100.0 | 100.0 | 93260_IBD Colitis 2 | 1.1 | 0.0 |
| 93776_Monocytes_LPS 50 ng/ml | 0.0 | 0.3 | 93261_IBD Crohns | 2.2 | 3.5 |
| 93581_Macrophages_resting | 0.6 | 3.7 | 735010_Colon_normal | 21.5 | 15.6 |
| 93582_Macrophages_LPS 100 ng/ml | 1.0 | 3.1 | 735019_Lung_none | 17.6 | 14.4 |
| 93098_HUVEC (Endothelial)_none | 0.0 | 0.0 | 64028-1_Thymus_none | 11.8 | 7.1 |
| 93099_HUVEC (Endothelial)_starved | 0.0 | 0.0 | 64030-1_Kidney_none | 33.7 | 20.3 |

### EQUIVALENTS

Although particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, and 22;
(b) a variant of a mature form of an amino acid sequence selected from the group consisting of SEQ ID NOS:2, 4, 6, 8, 10, 12, 14, 16, 18, 20, and 22, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, and 22; and
(d) a variant of an amino acid sequence selected from the group consisting of SEQ ID NOS:2, 4, 6, 8, 10, 12, 14, 16, 18, 20, and 22, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, and 22.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7,9, 11, 13, 15, 17, 19, and 21.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, and 22;
(b) a variant of a mature form of an amino acid sequence selected from the group consisting of SEQ ID NOS:2, 4, 6, 8, 10, 12, 14, 16, 18, 20, and 22, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, and 22;
(d) a variant of an amino acid sequence selected from the group consisting of SEQ ID NOS:2, 4, 6, 8, 10, 12, 14, 16, 18, 20, and 22, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, and 22, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, and 21.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, and21;
(b) a nucleotide sequence differing by one or more nucleotides from a nucleotide sequence selected from the group consisting of SEQ ID NOS:1, 3, 5, 7, 9, 11, 13, 15, 17, 19, and 21, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, and 21, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide;
and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a GPCRX-associated disorder, said method comprising administering to a subject in which such treatment or prevention is desired the polypeptide of claim 1 in an amount sufficient to treat or prevent said GPCRX-associated disorder in said subject.

27. The method of claim 26 wherein the disorder is selected from the group consisting of cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

28. The method of claim 26 wherein the disorder is related to cell signal processing and metabolic pathway modulation.

29. The method of claim 26, wherein said subject is a human.

30. A method of treating or preventing a GPCRX-associated disorder, said method comprising administering to a subject in which such treatment or prevention is desired the nucleic acid of claim 5 in an amount sufficient to treat or prevent said GPCRX-associated disorder in said subject.

31. The method of claim 30 wherein the disorder is selected from the group consisting of cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

32. The method of claim 30 wherein the disorder is related to cell signal processing and metabolic pathway modulation.

33. The method of claim 30, wherein said subject is a human.

34. A method of treating or preventing a GPCRX-associated disorder, said method comprising administering to a subject in which such treatment or prevention is desired the antibody of claim 15 in an amount sufficient to treat or prevent said GPCRX-associated disorder in said subject.

35. The method of claim 34 wherein the disorder is selected from the group consisting of diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject;
and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to cancers.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20 and 22, or a biologically active fragment thereof.

49. A method of treating a pathological state in a mammal, the method comprising administering to the mammal the antibody of claim 15 in an amount sufficient to alleviate the pathological state.
